SEQUENCE LISTING (38 sequences)

SEQ ID NO: 1 - protein, 5 aa, Artificial
  synthetic peptide
FVFLM

SEQ ID NO: 2 - protein, 5 aa, Artificial
  synthetic peptide
FVFAM

SEQ ID NO: 3 - protein, 5 aa, Artificial
  synthetic peptide
FVALM

SEQ ID NO: 4 - protein, 5 aa, Artificial
  synthetic peptide
FVFLA

SEQ ID NO: 5 - protein, 5 aa, Artificial
  synthetic peptide
FLVFI

SEQ ID NO: 6 - protein, 5 aa, Artificial
  synthetic peptide
FLMII

SEQ ID NO: 7 - protein, 5 aa, Artificial
  synthetic peptide
FLFVL

SEQ ID NO: 8 - protein, 5 aa, Artificial
  synthetic peptide
FLFVV

SEQ ID NO: 9 - protein, 5 aa, Artificial
  synthetic peptide
FLFLI

SEQ ID NO: 10 - protein, 5 aa, Artificial
  synthetic peptide
FLFFI

SEQ ID NO: 11 - protein, 5 aa, Artificial
  synthetic peptide
FLMFI

SEQ ID NO: 12 - protein, 5 aa, Artificial
  synthetic peptide
FMLLI

SEQ ID NO: 13 - protein, 5 aa, Artificial
  synthetic peptide
FIIMI

SEQ ID NO: 14 - protein, 5 aa, Artificial
  synthetic peptide
FLFCI

SEQ ID NO: 15 - protein, 5 aa, Artificial
  synthetic peptide
FLFAV

SEQ ID NO: 16 - protein, 5 aa, Artificial
  synthetic peptide
FVYLI

SEQ ID NO: 17 - protein, 5 aa, Artificial
  synthetic peptide
FAFLM

SEQ ID NO: 18 - protein, 5 aa, Artificial
  synthetic peptide
AVFLM

SEQ ID NO: 19 - protein, 417 aa, Artificial
  derived from AAT proteins
MPSSVSWGILLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDHEEAKKQINDYVEKGTQGKIVDLVKELDRDTVFALVNYIFFKGKWERPFEVKDTEDEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 20 - protein, 394 aa, Artificial Sequence
  derived from AAT proteins
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 21 - protein, 10 aa, Artificial Sequence
  derived from AAT proteins
GADLSGVTEE

SEQ ID NO: 22 - protein, 10 aa, Artificial Sequence
  derived from AAT proteins
APLKLSKAVH

SEQ ID NO: 23 - protein, 10 aa, Artificial Sequence
  derived from AAT proteins
TEAAGAMFLE

SEQ ID NO: 24 - protein, 10 aa, Artificial Sequence
  derived from AAT proteins
RIPVSIPPEV

SEQ ID NO: 25 - protein, 10 aa, Artificial Sequence
  derived from AAT proteins
KFNKPFVFLM

SEQ ID NO: 26 - protein, 10 aa, Artificial Sequence
  derived from AAT proteins
IEQNTKSPLF

SEQ ID NO: 27 - protein, 10 aa, Artificial Sequence
  derived from AAT proteins
MGKVVNPTQK

SEQ ID NO: 28 - protein, 10 aa, Artificial Sequence
  derived from AAT proteins
LSGVTEEAPL

SEQ ID NO: 29 - protein, 10 aa, Artificial Sequence
  derived from AAT proteins
KLSKAVHKAV

SEQ ID NO: 30 - protein, 10 aa, Artificial Sequence
  derived from AAT proteins
LTIDEKGTEA

SEQ ID NO: 31 - protein, 10 aa, Artificial Sequence
  derived from AAT proteins
AGAMFLERIP

SEQ ID NO: 32 - protein, 10 aa, Artificial Sequence
  derived from AAT proteins
VSIPPEVKFN

SEQ ID NO: 33 - protein, 10 aa, Artificial Sequence
  derived from AAT proteins
KPFVFLMIEQ

SEQ ID NO: 34 - protein, 10 aa, Artificial Sequence
  derived from AAT proteins
NTKSPLFMGK

SEQ ID NO: 35 - protein, 7 aa, Artificial Sequence
  derived from AAT proteins
VVNPTQK

SEQ ID NO: 36 - protein, 22 aa, Artificial Sequence
  derived from AAT proteins
LEAIPMSIPPEVKFNKPFVFLM

SEQ ID NO: 37 - protein, 20 aa, Artificial Sequence
  derived from AAT proteins
LEAIPMSIPPEVKFNKPFVF

SEQ ID NO: 38 - protein, 80 aa, Artificial Sequence
  derived from AAT proteins
GADLSGVTEEAPLKLSKAVHKAVLTIDEKGTEAAGAMFLERIPVSIPPEVKFNKPFVFLMIEQNTKSPLFMGKVVNPTQK

(19) United States
(12) Patent Application Publication
(10) Pub. No.: US 2010/0144630 A1
(43) Pub. Date: Jun. 10, 2010

(54) Compositions, methods and uses for inhibition and/or treatment of influenza infection

(76) Inventor: Leland Shapiro, Denver, CO (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/589,221

(22) Filed: Oct. 20, 2009

Related U.S. Application Data
(63) Continuation-in-part of application No. 12/586,818, filed on Sep. 29, 2009.
Application No. 12/586,818 is a continuation-in-part of application No. 11/044,224, filed on Jan. 28, 2005.
Application No. 11/044,224 is a continuation of application No. 09/518,098, filed on Mar. 3, 2000, now Pat. No. 6,849,605.
(60) Provisional application No. 60/137,795, filed on Jun. 3, 1999.
(60) Provisional application No. 60/123,168, filed on Mar. 5, 1999.
(60) Provisional application No. 60/123,167, filed on Mar. 5, 1999.

(51) Int. Cl. A61K 38/16; A61K 38/10; A61K 38/08; A61P 37/04; A61P 31/12; A61P 31/14; A61P 31/16; A61P 31/18; A61P 31/20; A61P 31/22

(52) U.S. Cl. 514/12; 514/17; 514/15; 514/16; 514/13

(57) ABSTRACT

Embodiments of the present invention illustrate methods and compositions for treating medical disorders. In certain embodiments, compositions and methods relate to reducing or inhibiting onset, transmission or development of a viral disorder.

### CROSS REFERENCE TO RELATED APPLICATIONS

This application is a continuation-in-part application of, and claims priority to, U.S. patent application Ser. No. 12/586,818 filed Sep. 28, 2009 which is a continuation-in-part application of U.S. patent application Ser. No. 11/044,224 filed Jan. 28, 2005, which is a continuation application of U.S. patent application Ser. No. 09/518,098, filed Mar. 3, 2000, which claims priority to U.S. Provisional Patent Application Ser. No. 60/137,795, filed Jun. 3, 1999 and U.S. Provisional Patent Application Ser. No. 60/123,167, filed Mar. 5, 1999. All applications are incorporated herein by reference in their entirety for all purposes.

### FIELD

Embodiments of the present invention report compositions, methods and uses for alpha-1 antitrypsin (α1-antitrypsin, AAT) and AAT-derived or associated molecules for prevention of, or treatment for, viral infections. In certain embodiments, molecules associated with AAT for prevention of, or treatment for, viral infections can be peptides derived from about the last 80 amino acids in the carboxy-terminus of naturally occurring or native AAT. Other embodiments relate to compositions and methods for prevention or treatment of medical conditions associated with viral infections. Yet other embodiments report compositions and methods for prevention or modulation of transmission of a virus from cell to cell and/or from host to host.

### BACKGROUND

Normal human plasma concentration of AAT ranges from 1.3 to 3.5 mg/ml. Under certain conditions, AAT can behave as an acute phase reactant and increase 3-4-fold during host response to inflammation and/or tissue injury or dramatic change such as with pregnancy, acute infection, and tumors. AAT easily diffuses into tissue spaces and forms a 1:1 complex with target proteases, principally neutrophil elastase. Other enzymes such as trypsin, chymotrypsin, cathepsin G, plasmin, thrombin, tissue kallikrein, and factor Xa can also serve as substrates. The enzyme/inhibitor complex is then removed from circulation by binding to serpin-enzyme complex (SEC) receptor and catabolized by the liver and spleen.

AAT is approved for the clinical therapy of protease imbalance. Therapeutic AAT has been commercially available since the mid 1980's and is prepared by various purification methods.

### Human Immunodeficiency Virus (HIV)

Previous research has shown that replication of HIV requires protease activity amongst other activities for the cleavage of gag-pol precursor proteins. This enzymatic activity is similar to activity of renin-aspartyl protease produced by the kidney.

### Influenza Virus

Influenza is an orthomyxovirus. Three genera, types A, B, and C of influenza currently exist. Types A and B are the most clinically significant, causing mild to severe respiratory illness. Type A viruses exist in both human and animal populations, with significant avian and swine reservoirs. Although relatively uncommon, it is possible for nonhuman influenza A strains to infect humans by jumping from their natural host. In one specific example, the highly lethal Hong Kong avian influenza outbreak in humans in 1997 was due to an influenza A H5N1 virus that was an epidemic in the local poultry population at that time. In this case, the virus killed six of the 18 patients shown to have been infected.

Annual influenza A virus infections have a significant impact on humanity both in terms of death, between 500,000 and 1,000,000 worldwide each year and economic impact resulting from direct and, indirect loss of productivity during infection.

In 2009, a flu pandemic erupted. The virus isolated from patients in the United States was found to be made up of genetic elements from four different flu viruses—North American Mexican influenza, North American avian influenza, human influenza, and swine influenza virus typically found in Asia and Europe. This new strain appears to be a result of reassortment of human influenza and swine influenza viruses, in all four different strains of subtype H1N1.

### SUMMARY

Embodiments of the present invention report compositions, methods and uses for alpha-1 antitrypsin (α1-antitrypsin, AAT) and AAT-derived or associated molecules for prevention of, or treatment for, viral infections. In certain embodiments, molecules associated with AAT for prevention of, or treatment for, viral infections can be peptides derived from about the last 80 amino acids of the carboxy-terminus of naturally occurring or native AAT. Other embodiments relate to compositions and methods for prevention or treatment of medical conditions associated with viral infections.

Some embodiments of the present invention report compositions of use for reducing onset or treating viral-related disorders. In accordance with these embodiments, the disorder may include, but is not limited to, HIV infection, AIDS (acquired immunodeficiency syndrome), influenza virus infection, hepatitis virus infection, Herpes virus infection, human papilloma virus infection, Variola major virus (small pox), Lassa fever virus infection, avian flu, AIDS Related Complex, Chickenpox (Varicella), Common cold, Cytomegalovirus Infection, Colorado tick fever, Dengue fever, Ebola haemorrhagic fever, Hand, foot and mouth disease, Hepatitis, Herpes simplex, Herpes zoster, HPV, Influenza (Flu), Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease, Yellow fever, and a combination thereof.

Certain embodiments concern compositions for treating a subject having or suspected of developing a viral-related disorder. In accordance with these embodiments, a composition for modulating the onset or treating a viral-related disorder can include, alpha-1 antitrypsin (AAT), AAT-associated molecules or AAT-derived carboxy-terminal peptide molecules, for example carboxy-terminal peptides derived from the last 80 amino acids of SEQ ID NO:20, naturally occurring AAT. Native AAT is a glycoprotein of MW 51,000 with 394 amino acids and 3 oligosaccharide side chains. Human AAT was named anti-trypsin because of its initially discovered ability to inactivate pancreatic trypsin. Human AAT is a single polypeptide chain with no internal disulfide bonds and only a single cysteine residue normally intermolecularly disulfide-linked to either cysteine or glutathione. Certain embodiments can include peptides having amino acid lengths of 5, 10, 15, 20 or more of contiguous amino acids derived from the last 80 AA of SEQ ID NO:20 beginning at amino acid 315 and ending at amino acid 394. Other embodiments can include analogs of peptides having amino acid lengths of 5, 10, 15, 20 or more of contiguous amino acids derived from the last 80 AA of SEQ ID NO:20. In accordance with these embodiments, peptides contemplated herein may include mixtures of peptides of various amino acid sequence lengths and activities, derived from the carboxy-terminal last 80 AA of SEQ ID NO:20 beginning at residue 315. In other embodiments, the composition may further include, but is not limited to an anti-inflammatory agent, an immunosuppressive agent, an immunomodulatory agent, an anti-microbial agent, an anti-viral agent, an anti-bacterial agent, and a combination thereof.

The amino acid sequence of SEQ ID NO:20 is represented by:

```
Glu Asp Pro Gln Gly Asp Ala Ala Gln Lys Thr Asp Thr Ser His His
1 5 10 15
Asp Gln Asp His Pro Thr Phe Asn Lys Ile Thr Pro Asn Leu Ala Glu
                          20 25 30
Phe Ala Phe Ser Leu Tyr Arg Gln Leu Ala His Gln Ser Asn Ser Thr
                      35 40 45
Asn Ile Phe Phe Ser Pro Val Ser Ile Ala Thr Ala Phe Ala Met Leu
  50 55 60
Ser Leu Gly Thr Lys Ala Asp Thr His Asp Glu Ile Leu Glu Gly Leu
65 70 75 80
Asn Phe Asn Leu Thr Glu Ile Pro Glu Ala Gln Ile His Glu Gly Phe
                  85 90 95
Gln Glu Leu Leu Arg Thr Leu Asn Gln Pro Asp Ser Gln Leu Gln Leu
            100 105 110
Thr Thr Gly Asn Gly Leu Phe Leu Ser Glu Gly Leu Lys Leu Val Asp
        115 120 125
Lys Phe Leu Glu Asp Val Lys Lys Leu Tyr His Ser Glu Ala Phe Thr
    130 135 140
Val Asn Phe Gly Asp His Glu Glu Ala Lys Lys Gln Ile Asn Asp Tyr
145 150 155 160
Val Glu Lys Gly Thr Gln Gly Lys Ile Val Asp Leu Val Lys Glu Leu
                  165 170 175
Asp Arg Asp Thr Val Phe Ala Leu Val Asn Tyr Ile Phe Phe Lys Gly
            180 185 190
Lys Trp Glu Arg Pro Phe Glu Val Lys Asp Thr Glu Asp Glu Asp Phe
        195 200 205
His Val Asp Gln Val Thr Thr Val Lys Val Pro Met Met Lys Arg Leu
    210 215 220
Gly Met Phe Asn Ile Gln His Cys Lys Lys Leu Ser Ser Trp Val Leu
225 230 235 240
Leu Met Lys Tyr Leu Gly Asn Ala Thr Ala Ile Phe Phe Leu Pro Asp
                  245 250 255
Glu Gly Lys Leu Gln His Leu Glu Asn Glu Leu Thr His Asp Ile Ile
            260 265 270
Thr Lys Phe Leu Glu Asn Glu Asp Arg Arg Ser Ala Ser Leu His Leu
        275 280 285
Pro Lys Leu Ser Ile Thr Gly Thr Tyr Asp Leu Lys Ser Val Leu Gly
    290 295 300
Gln Leu Gly Ile Thr Lys Val Phe Ser Asn Gly Ala Asp Leu Ser Gly
305 310 315 320
Val Thr Glu Glu Ala Pro Leu Lys Leu Ser Lys Ala Val His Lys Ala
                  325 330 335
Val Leu Thr Ile Asp Glu Lys Gly Thr Glu Ala Ala Gly Ala Met Phe
            340 345 350
Leu Glu Arg Ile Pro Val Ser Ile Pro Pro Glu Val Lys Phe Asn Lys
          355 360 365
Pro Phe Val Phe Leu Met Ile Glu Gln Asn Thr Lys Ser Pro Leu Phe
    370 375 380
Met Gly Lys Val Val Asn Pro Thr Gln Lys
                                        390
```

The last 80 AA of SEQ ID NO:20 beginning at amino acid 315 and ending at amino acid 394 SEQ ID NO:38 is represented by:

GADLSGVTEEAPLKLSKAVHKAVLTIDEKGTEAAGAMFLERIPVSIPPEV
KFNKPFVFLMIEQNTKSPLFMGKVVNPTQK

Some embodiments reported herein concern methods of treating a subject having a viral infection including, but not limited to, administering to the subject in need of such a treatment a therapeutically effective amount of a composition comprising alpha-1 antitrypsin associated molecules or alpha-1 antitrypsin-like molecules. In accordance with these embodiments, the disorder can be HIV infection, AIDS (acquired immunodeficiency syndrome), influenza virus infection, hepatitis virus infection, Herpes virus infection, human papilloma virus infection, Variola major virus (small pox), Lassa fever virus infection, avian flu, AIDS Related Complex, Chickenpox (Varicella), Common cold, Cytomegalovirus Infection, Colorado tick fever, Dengue fever, Ebola haemorrhagic fever, Hand, foot and mouth disease, Hepatitis, Herpes simplex, Herpes zoster, HPV, Influenza (Flu), Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease, Yellow fever, and a combination thereof.

In certain embodiments, a virus can include an influenza virus infection, for example, influenza type A, B or C or subtype or strain thereof. Some embodiments include, but are not limited to, influenza A, H1N1 subtype and strains. Other influenza A viruses may include, but are not limited to, H2N2, which caused Asian Flu in 1957; H3N2, which caused Hong Kong Flu in 1968; H5N1, a current pandemic threat; H7N7, which has unusual zoonotic potential; H1N2, endemic in humans and pigs; H9N2; H7N2; H7N3, H10N7 or combinations thereof.

Compositions contemplated herein may further include an agent selected from the group consisting of an anti-inflammatory agent, an immunosuppressive agent, an immunomodulatory agent, an anti-viral agent, an anti-pathogenic agent, an anti-bacterial agent, a reverse transcriptase inhibitor, a protease inhibitor, and a combination thereof.

In certain embodiments, compositions herein can be administered orally, systemically, via an implant, intravenously, intradermally, topically, intrathecally, intravaginally, as a suppository, subcutaneously, by inhalation, nasally, or by other means known in the art or a combination thereof.

Methods of treatment contemplated herein can include reducing incidence or onset of infection in a subject exposed to a virus or suspected of having been exposed to a virus.

Certain methods of treatment further concern reducing or eliminating one or more symptom associated with a infectious disorder including, but not limited to, peripheral edema, organ edema hemorrhage, ischemia, vascular permeability, apoptosis, hemorrhage, ischemia or a combination thereof.

In a more particular embodiment, a viral medical disorder can include an influenza infection. In accordance with these embodiments, the influenza infection can include influenza A or influenza B infection.

In certain embodiments, compositions and methods disclosed herein can be used to modulate incidence of viral-associated indications or infections. In accordance with these embodiments, modulating incidence of viral-associated indications or infections is on the order of about 10-20%, or about 30-40%, or about 50-60%, or about 75-90% or about 91-100% reduction or inhibition. In other embodiments, compositions and methods disclosed herein can be used to modulate lung accumulation of influenza by administering to a subject compositions disclosed herein. For example, a subject having or suspected of developing a viral infection of the lung may be treated with AAT or AAT-derived peptide compositions. In one embodiment, a subject may be treated with a composition having a peptide derived from AAT or the last 80 amino acids of the carboxyterminus of AAT. In other embodiments, these compositions may include FVFLM (SEQ ID NO:1), FVYLI (SEQ ID NO:16) or an analog thereof.

In certain embodiments, AAT-associated molecules used in the methods and compositions herein can include, but are not limited to, compositions of SEQ ID NO:20, naturally occurring AAT (394 AA length molecule making up approximately 90% of AAT isolated from serum), Aralast™ (Baxter), Zemaira™ (Aventis Behring), Prolastin™ (Bayer), Aprotonin™ or Trasylol™ (Bayer Pharmaceutical Corporation), Ulinistatin™ (Ono Pharmaceuticals, Inc.), and inhalation and/or injectible AAT (Kamada, Ltd., Israel), or any combination thereof.

In other embodiments, the anti-inflammatory compound or immunomodulatory drug can include, but is not limited to, interferon; interferon derivatives comprising betaseron, β-interferon; prostane derivatives comprising iloprost, cicaprost; glucocorticoids comprising cortisol, prednisolone, methylprednisolone, dexamethasone; immunsuppressives comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; TNF-antibodies; soluble receptors of interleukines, other cytokines, T-cell-proteins; antibodies against receptors of interleukines, other cytokines, T-cell-proteins; and calcipotriols and analogues thereof taken either alone or in combination.

In certain embodiments, compositions for administration to a subject can be in a range of between about 10 ng and about 10 mg per ml or mg of the formulation (e.g about 2 of about 3 mg/ml). In some embodiments, compositions for administration to a subject can be in a range of between about 50 ng and about 200 ng per ml. A therapeutically effective amount of AAT-associated or AAT-derived molecule or drug that have similar activities as AAT or peptide compositions may be measured in molar concentrations and may range between about 1 nM and about 10 mM. Formulations are also contemplated in combination with a pharmaceutically or cosmetically acceptable carrier. Dose can be established by well known routine clinical trials and healthcare providers without undue experimentation.

In certain embodiments, the subject or mammal is a human.

In other embodiments, the subject or mammal can be a domesticated or a non-domesticated mammal.

In certain embodiments, synthetic and/or naturally occurring peptides may be used in compositions and methods herein for example. Homologues, natural peptides, with sequence homologies to AAT including peptides directly derived from cleavage of AAT may be used or other peptides such as, peptides that have AAT-like activity. Other peptidyl derivatives, e.g., aldehyde or ketone derivatives of such peptides are also contemplated herein. Without limiting to AAT and peptide derivatives of AAT, compounds like oxadiazole, thiadiazole and triazole peptoids and substances can include, but are not limited to, certain phenylenedialkanoate esters, CE-2072, UT-77, and triazole peptoids. Examples of analogues are TLCK (tosyl-L-lysine chloromethyl ketone) or TPCK (tosyl-L-phenylalanine chloromethyl ketone).

### BRIEF DESCRIPTION OF THE DRAWINGS

The following drawings form part of the present specification and are included to further demonstrate certain embodiments disclosed herein. Embodiments may be better understood by reference to one or more of these drawings in combination with the detailed description of specific embodiments presented herein.

FIG. 1 illustrates the effect of AAT on HIV production in PBMC as performed without pre-incubation.

FIG. 2 illustrates the effect of AAT on HIV production in PBMC as performed with pre-incubation.

FIG. 3 illustrates the effect of AAT on HIV production in MAGI cells.

FIG. 4 illustrates the effect of FVYLI (SEQ. ID NO. 16) peptide on HIV production in MAGI cells.

FIG. 5 illustrates the effect of AAT on HIV production in U1 cells upon induction with IL-18.

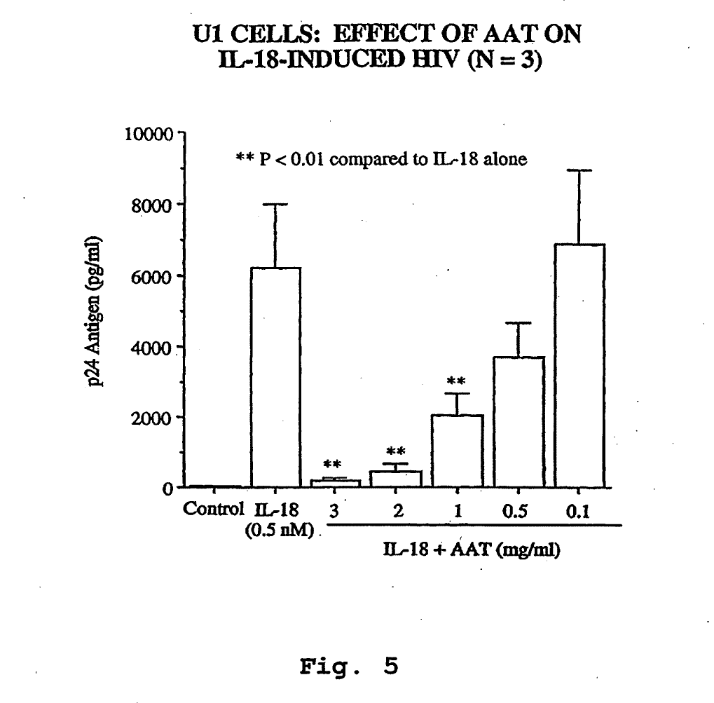

FIG. 6 illustrates the lack of effect of Prolastin on HIV production in U1 cells upon induction with IL-18.

FIG. 7 illustrates the effect of AAT on HIV production in U1 cells upon induction with IL-6.

FIG. 8 illustrates the effect of AAT on HIV production in U1 cells upon induction with TNF.

FIG. 9 illustrates the effect of AAT on HIV production in U1 cells upon induction with LPS.

FIG. 10 illustrates the effect of AAT on HIV production in U1 cells upon induction with NaCl.

FIG. 11 illustrates the effect of AAT-mimicking drug on HIV production in U1 cells upon induction with IL-18.

FIG. 12 illustrates the effect of AAT on viability and number of U1 cells.

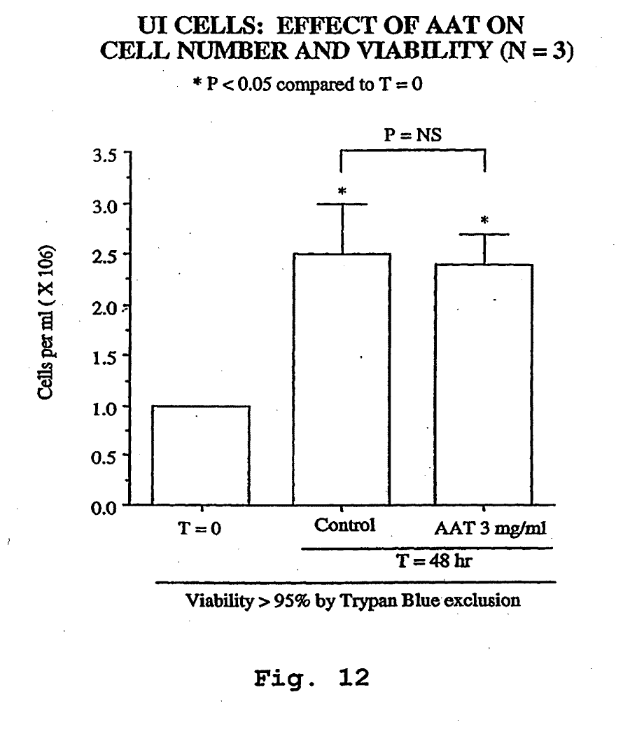

FIG. 13 illustrates the p24 antigen output of HIV when grown in normal or AAT-deficient whole blood.

FIG. 14 illustrates the effect of AAT and AAT-mimicking drug (CE 2072) in reducing IL-18-induced NF-κB activation.

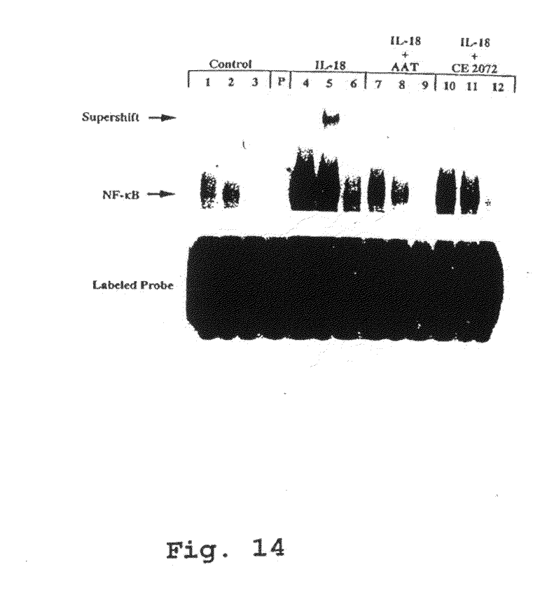

FIGS. 15A and 15B represents an exemplary histogram of the effects of AAT (15A left panel, solid bars) or HI AAT (15A right panel, open bars) at 0, 6, 4, 2 and 1 mg/ml on HIV production represented by p24 production (pg/ml) in stimulated U1 cells. FIG. 15B represents an exemplary histogram of the effects of AAT (5 mg/ml, 0.8 mg/ml) or HI AAT (striped bar, 5 mg/ml, 0.8 mg/ml) on HIV production represented by p24 production (pg/ml) in stimulated U1 cells.

FIG. 16 represents a graphic illustration of the 1918 influenza outbreak and resulting increase in mortality rate.

FIG. 17 represents a graphic illustration of the effect of increasing amounts of AAT on flu production at Day 2 in vitro compared to controls.

FIG. 18 represents fluorescence detection of flu in an exemplary in vitro experiment, A) represents flu alone and B) represents influenza in the presence of a composition disclosed herein.

FIG. 19 represents an exemplary graphic representation of correlation of risk of influenza infection over time in subjects having reduced levels of AAT compared to a normal population.

FIG. 20 represents an exemplary mouse model of an in vivo assay of influenza (H1N1) infection in the presence and absence of a composition disclosed herein and mouse survival over several days.

FIG. 21 represents an exemplary in vivo assay of effects of AAT on infection of mice with influenza H1N1.

FIG. 22 represents a pathology section of mice lung comparing pneumonia infiltrates in the presence or absence of AAT. Lobar pneumonia (A) with severe mixed acute and chronic inflammatory infiltrate (B) in wild type mouse. Characteristic patchy bronchopneumonia (C) with mild mixed acute and chronicinflammatory infiltrate (D) in transgenic α-1-antitrypsin overexpressing mouse and (E) inset.

FIG. 23 represents weight loss in transgenic mice expressing human AAT in the lungs compared to wild type control mice.

FIG. 24 represents caspase-1 levels in wild-type (control) compared to transgenic mice expressing human AAT in the lungs, days after exposure to influenza.

### DESCRIPTION OF ILLUSTRATIVE EMBODIMENTS

### Definitions

Terms that are not otherwise defined herein are used in accordance with their plain and ordinary meaning.

As used herein, “a” or “an” may mean one or more than one of an item.

As used herein, “about” can mean plus or minus 10%, for example, about 10 minutes can mean from 9 to 11 minutes.

As used herein “analog of alpha-1-antitrypsin” may mean a compound having alpha-1-antitrypsin-like activity like serine protease inhibitor activity or cytokine production inhibition or other anti-viral activity. In one embodiment, an analog of alpha-1-antitrypsin is a functional derivative of alpha-1-antitrypsin. In some embodiments, an analog of alpha-1-antitrypsin is a compound with no significant serine protease inhibitor activity.

As used herein “alpha-1 antitrypsin-associated or AAT-associated molecules” can mean, e.g. molecules or agents associated with partial purification of AAT other than AAT itself. These associated molecules typically comprise about 10-30 percent of the partially purified commercial product.

As used herein “alpha-1 antitrypsin-derived or AAT-derived molecules” can mean, e.g. molecules or peptide fragments derived from AAT such as the last 80 amino acids of the carboxyterminus of SEQ ID NO:20 or fragments thereof.

As used herein “immunomodulatory drugs or agents”, can mean, e.g., agents capable of acting on the immune system, directly or indirectly, e.g., by stimulating or suppressing a cellular activity of a cell in the immune system, e.g., T-cells, B-cells, macrophages, or antigen presenting cells (APC, dendritic cells), or by acting upon components outside the immune system which, in turn, stimulate, suppress, or modulate the immune system, e.g. cytokines, hormones, receptor agonists or antagonists, and neurotransmitters; immunomodulators (e.g., immunosuppressants or immunostimulants).

### DETAILED DESCRIPTION

In the following sections, various exemplary compositions and methods are described in order to detail various embodiments of the invention. It will be obvious to one skilled in the art that practicing the various embodiments does not require the employment of all or even some of the details outlined herein, but rather that concentrations, times and other details may be modified based on routine experimentation or knowledge in the art. In some cases, well known methods, or components have not been included in the description.

Embodiments herein provide for methods and compositions for treating a subject having or suspected of developing a viral-derived disorder. In accordance with these embodiments, the disorder may include, but is not limited to, a viral infection. Other embodiments provide for compositions, methods and uses for modulating transmission of a virus from cell to cell and/or subject host to subject host (e.g. administering a composition to an infected cell/tissue or infected host) in comparison to a subject not administered a composition disclosed herein (e.g. a control subject).

Certain embodiments concern compositions for modulating incidence of (onset of) or treating a subject suspected of developing or having a viral-derived disorder. In accordance with these embodiments, the composition may include, alpha-1 antitrypsin or alpha-1 antitrypsin-associated or derived molecule. In other embodiments, the composition may further include, but is not limited to an anti-inflammatory agent, an immunosuppressive agent, an immunomodulatory agent, an anti-microbial agent, an anti-viral agent, an anti-bacterial agent, and a combination thereof. In certain embodiments, compositions may include one or more peptide or mixture thereof of 5, 6, 7, 8, 9, 10, 11, 12, 13, 14, 15, 16, 17, 18, 19, 20 or more of amino acids 315 to 394 of SEQ ID NO:20. Amino acids or residues 315 to 394 of SEQ ID NO:20 can be represented as one-letter amino acid codes as follows:

(SEQ. ID NO. 38)

GADLSGVTEEAPLKLSKAVHKAVLTIDEKGTEAAGAMFLERIPVSIPPEV
KFNKPFVFLMIEQNTKSPLFMGKVVNPTQK.

Some embodiments of the present invention can include a mixture of one or more peptide comprising FVFLM (SEQ. ID NO. 1), or analogs of FVFLM: FVFAM (SEQ. ID NO. 2), FVALM (SEQ. ID NO. 3), FVFLA (SEQ. ID NO. 4), FLVFI (SEQ. ID NO. 5), FLMII (SEQ. ID NO. 6), FLFVL (SEQ. ID NO. 7), FLFVV (SEQ. ID NO. 8), FLFLI (SEQ. ID NO. 9), FLFFI (SEQ. ID NO. 10), FLMFI (SEQ. ID NO. 11), FMLLI (SEQ. ID NO. 12), FIIMU (SEQ. ID NO. 13), FLFCI (SEQ. ID NO. 14), FLFAV (SEQ. ID NO. 15), FVYLI (SEQ. ID NO. 16), FAFLM (SEQ. ID NO. 17), AVFLM (SEQ. ID NO. 18), and combination thereof.

Other embodiments can include a mixture of one or more peptide selected from peptidyl derivatives from the last 80 carboxy terminal amino acids of SEQ ID NO:20 including, but not limited to, GADLSGVTEE (SEQ ID NO:21); APLKLSKAVH (SEQ ID NO:22); KAVLTIDEKG (SEQ ID NO:22); TEAAGAMFLE (SEQ ID NO:23); RIPVSIPPEV (SEQ ID NO:24); KFNKPFVFLM (SEQ ID NO:25); IEQNTKSPLF (SEQ ID NO:26); MGKVVNPTQK (SEQ ID NO:27); LSGVTEEAPL (SEQ. ID NO. 28); KLSKAVHKAV (SEQ. ID NO. 29); LTIDEKGTEA (SEQ. ID NO. 30); AGAMFLERIP (SEQ. ID NO. 31); VSIPPEVKFN (SEQ. ID NO. 32); KPFVFLMIEQ (SEQ. ID NO. 33); NTKSPLFMGK (SEQ. ID NO. 34); VVNPTQK (SEQ. ID NO. 35); LEAIPMSIPPEVKFNKPFVFLM (SEQ ID NO: 36); and LEAIPMSIPPEVKFNKPFVF (SEQ ID NO: 37), or any combination thereof. It is contemplated that the AAT-derived peptides from the carboxyterminus recited for use in the compositions and methods herein are also intended to include any and all of those specific AAT peptides other than the 10 amino acid AAT peptides of SEQ ID NO. 20 depicted supra. For example, while AAT peptides amino acids 315-324, amino acids 325-334, amino acids 335-344, etc of SEQ ID NO. 20 have been enumerated herein, it is intended that the scope of the compositions and methods of use of same specifically include all of the possible combinations of AAT peptides such as amino acids 316-325, amino acid 317-326, 318-327, etc. of SEQ ID NO. 20, as well as any and all AAT peptide fragments corresponding to select amino acids of SEQ ID NO. 20, without actually reciting each specific AAT peptide of SEQ ID NO. 20 therewith. Thus, by way of illustration, and not by way of limitation, Applicants are herein entitled to possession of compositions based upon any and all AAT peptide variants based upon the amino acid sequence depicted in SEQ ID NO. 20 and use of such compositions in the methods of the present invention.

In certain embodiments, AAT-associated molecules used in the methods and compositions herein can include, but are not limited to, compositions of SEQ ID NO:20, naturally occurring AAT (394 AA length molecule making up approximately 90% of AAT isolated from serum), or other AAT compositions such as, Aralast™ (Baxter), Zemaira™ (Aventis Behring), Prolastin™ (Bayer), Aprotonin™ or Trasylol™ (Bayer Pharmaceutical Corporation), Ulinistatin™ (Ono Pharmaceuticals, Inc.), and inhalation and/or injectible AAT (Kamada, Ltd., Israel), or any other commercially available AAT compositions or any combination thereof.

Other embodiments concern methods of treating a subject with a viral disorder including administering to the subject in need of such a treatment a therapeutically effective amount of a composition including but not limited to alpha-1 antitrypsin or alpha-1 antitrypsin-derived peptide composition. In accordance with these embodiments, the disorder can be a viral infection.

### Influenza

Influenza (Flu) infection is a continuing concern as a public health problem and potential bioweapon. In the USA, 10-20% of the population are infected each year with influenza (30-60 million people), 114,000 are hospitalized, 36,000 patients die, and the cost to the USA economy is $3-15 billion. The emergence of the “Swine” Flu and human infection with “bird” Flu (60% mortality) has raised fears of an influenza pandemic with the possibility of 500 million deaths.

Because processing of influenza hemagglutinin (HA) for infection is required, effects of circulating serine protease inhibitor, alpha-1-antitrypsin (AAT) on influenza processing was assessed. AAT was demonstrated to inhibit primary monkey kidney cell infection with an H1N1 influenza strain in second and third-day cultures. Suppression of influenza infection in vitro was observed with a synthetic inhibitor of serine proteases. In one model, lung inflammatory molecules, (e.g. MIP-2 and IL-1α) were reduced in special human AAT (hAAT)-expressing mice compared to control mice at an early stage of analysis. Lung caspase-3 activity was 67% lower in these hAAT-carrying mice compared to control mice. Using an influenza-pneumonia model, it was observed that there were significant reductions in weight loss (morbidity) and mortality in AAT-carrying mice as compared to controls. In a lung transplant cohort, genetic AAT deficiency (N=28) was a significant risk factor for influenza infection.

In some embodiments, physiologically relevant doses (e.g. 3 or 1 mg/mL) of AAT or an AAT-derived molecule may be used to decrease influenza in a subject. Data presented herein suggest that one mechanism that compositions disclosed inhibit influenza is by inhibiting HA cleavage of initial infecting particles as well as newly emergent virions, rendering reduced ability to initiate further rounds of infection. In other embodiments, viral shedding from infected cell's of a subject may be modulated compared to a control by introducing a composition disclosed herein to a subject having an infection thereby reducing shedding of virus from the infected cell. In some embodiments, viral shedding can be reduced by administering an AAT composition or derivative thereof to a subject in need thereof compared to a control not receiving such a composition.

In certain embodiments, a medical disorder can include a viral infection for example, influenza such as influenza A, B or C. In accordance with these embodiments, a subject having been exposed to or having an influenza infection can be administered a therapeutically effective amount of a composition described herein. In some embodiments, a composition or pharmaceutically acceptable composition can include, but is not limited to, naturally occurring AAT (SEQ ID NO:20) or one or more peptides derived from the last 80 AA of the carboxyterminus of AAT (SEQ ID NO:20).

In other embodiments, a composition disclosed herein may be used to modulate or eliminate intracellular movement of a virus from the nucleus to the cytoplasm of an infected cell and/or reduce or eliminate extracellular excretion of a virus outside of the cell. Thus, administering a composition disclosed herein to a subject having a viral infection may reduce or eliminate transmission of the virus to another cell in that subject and/or reduce or eliminate transmission of the virus to another subject. In certain embodiments, the virus may be influenza virus. In other embodiments, a compositions may include naturally occurring AAT (SEQ ID NO:20) or one or more peptides derived from the last 80 AA of the carboxyterminus of AAT (SEQ ID NO:20). In accordance with these embodiments, a composition may comprise one or more of FVFLM (SEQUENCE ID NO. 1), an analog of FVFLM comprising FVYLI (SEQUENCE ID NO. 16), GADLSGVTEE (SEQ ID NO:21); KAVLTIDEKG (SEQ ID NO:22); TEAAGAMFLE (SEQ ID NO:23); RIPVSIPPEV (SEQ ID NO:24); KFNKPFVFLM (SEQ ID NO:25); IEQNTKSPLF (SEQ ID NO:26); MGKVVNPTQK (SEQ ID NO:27); LSGVTEEAPL (SEQ. ID NO. 28); KLSKAVHKAV (SEQ. ID NO. 29); LTIDEKGTEA (SEQ. ID NO. 30); AGAMFLERIP (SEQ. ID NO. 31); VSIPPEVKFN (SEQ. ID NO. 32); KPFVFLMIEQ (SEQ. ID NO. 33); NTKSPLFMGK (SEQ. ID NO. 34); VVNPTQK (SEQ. ID NO. 35); LEAIPMSIPPEVKFNKPFVFLM (SEQ ID NO: 36); LEAIPMSIPPEVKFNKPFVF (SEQ ID NO: 37) or a mixture thereof.

### Human Immunodeficiency Virus (HIV)

In certain embodiments, a viral disorder can include a viral infection for example, HIV or AIDS. In certain embodiments, compositions to prevent or treat HIV can include, but are not limited to, AAT and/or AAT-derived peptides from the last 80 amino acids of SEQ ID NO:20. In accordance with these embodiments, methods herein may concern treating a subject having HIV infection or modulating incidence of infection of a subject having been exposed to HIV by administering to the subject in need of such a treatment a therapeutically effective amount of a composition including, but not limited to, AAT and/or AAT-derived peptides from the last 80 amino acids of SEQ ID NO:20. Another embodiment includes regulating cellular infection by the virus in a subject by administering one or more compositions detailed herein.

A treatment contemplated herein for any viral-related disorder may include a treatment administered to a subject in need thereof multiple times daily, twice daily, daily, bi-weekly, weekly or other treatment regimen.

In addition, a treatment for a subject having an HIV infection can also include any other treatment known in the art. Other treatments can include, but are not limited to, anti-viral compounds, anti-HIV compounds, reverse transcriptase inhibitor and a combination thereof.

In certain embodiments, methods of treatment contemplated herein can be used for modulating reducing or preventing delivery of viral nucleic acid molecules into cells of a mammalian host, as well as, methods for reducing or preventing the exit of a virion particle from a mammalian host harboring an agent of a viral infection. Thus, treatments contemplated herein may both reduce infection in a mammalian host but may also reduce or prevent spread of the infection. In accordance with these methods, a post-exposure prophylaxis can be administered to a subject in need of such a treatment in order to block establishment of productive infection in a mammal exposed to HIV-contaminated fluids. Fluids contemplated to harbor HIV can include, for example, blood, saliva, semen, sweat, urine, vaginal secretion, tears, and other body fluids. In other embodiments, these methods and treatment compositions may be effective in reducing or preventing mother-to-child HIV transmission during pregnancy.

It is contemplated herein that assays for assessing the various activities of AAT or AAT-derived peptides can be used. In one particular embodiment, AAT and similarly active compounds may be identified by a series of assays wherein a compound will exhibit anti-inflammatory activity or anti-viral activity (e.g. viral infection) versus a control in an assay.

Other viral infections contemplated herein include, but are not limited to, viral infections that are caused/facilitated at least in part by a deficiency in AAT levels or by a dysfunction of AAT. Clinical conditions and viral infections resulting from uncontrolled AAT activity, other than serine protease inhibitor activity, are also within the scope herein.

Other agents are contemplated of use in combination with compositions of AAT and/or one or more peptide derived from the last 80 AA of the carboxyterminus of SEQ ID NO:20. In one embodiment, a method for treating HIV infection in a subject can include administering a therapeutically effective combination of (a) one or more compounds disclosed herein and (b) one or more compounds selected from the group consisting of HIV reverse transcriptase inhibitors and HIV protease inhibitors. Accordingly reverse transcriptase inhibitor can be selected from a group including nucleoside RT inhibitors: Retrovir (AZT/zidovudine; Glaxo Wellcome); Combivir (Glaxo Wellcome); Epivir (3TC, lamivudine; Glaxo Wellcome); Videx (ddl/didanosine; Bristol-Myers Squibb); Hivid (ddC/zalcitabine; Hoffmann-LaRoche); Zerit (d4T/stavudine; Bristol-Myers Squibb); Ziagen (abacavir, 1592U89; Glaxo Wellcome); Hydrea (Hydroxyurea[HO; nucleoside RT potentiator from Bristol-Myers Squibb) or Non-nucleoside reverse transcriptase inhibitors (NNRTIs): Viramune (nevirapine; Roxane Laboratories); Rescriptor (delavirdine; Pharmacia & Upjohn); Sustiva (efavirenz, DMP-266; DuPont Merck); Preveon (adefovir dipivoxil, bis-POM PMEA; Gilead). Protease inhibitors (PI's) are selected from Fortovase (saquinavir; Hoffmann-La Roche); Norvir (ritonavir; Abbott Laboratories); Crixivan (indinavir; Merck & Company); Viracept (nelfinavir; Agouron Pharmaceuticals); Angenerase (amprenavir/141W94; GlaxoWellcome), VX-478, KNI-272, CGP-61755, and U-103017.

Also contemplated is a method of preventing acquired or congenital deficiency of functional endogenous AAT levels in a subject susceptible to a viral infection that is mediated by AAT activity. In accordance with these methods, an effective amount of naturally occurring AAT or AAT-peptide derivative from the carboxy-terminus of SEQ ID NO:20 and another agent, such as, a thrombolytic agent such as tissue plasminogen activator, urokinase, streptokinase, or combinations or complexes thereof can be administered to the subject. The pharmaceutical composition may be one or more peptides in combination with other anti-viral compounds.

### Cytomegliovirus (CMV)

Cytomegalovirus (CMV) has a surface molecule HCMV gB that participates in viral entry into cells. A genetically engineered AAT variant, α1-PDX, was designed to confer inhibitory activity against furin. Extracellular α1-PDX blocked the production of infectious CMV in vitro, and the CMV inhibition was associated with reduced proteolytic activation of HCMV gB. Antiviral effect of AAT and of the genetically-engineered variant α1-PDX suggest a role for AAT in control of Influenza A and CMV production in vivo. In certain embodiments, it is contemplated that disclosed compositions and methods can be used to treat a subject having or exposed to CMV or influenza with a therapeutically effective amount of AAT and/or one or more AAT-derived peptides.

A genetic defect in humans can cause AAT deficiency in these subjects. Structurally abnormal AAT accumulates within liver cells, which are the primary source of circulating AAT. An associated defect in secretion from the liver results in serum concentrations of <15% of normal. This mutation affects 70,000-100,000 persons in the United States. Thus, patients having such a deficiency are more prone to an infection than a subject having normal levels of AAT and no genetic defect. Thus, some embodiments of the present invention contemplate treating a patient having a genetic deficiency related to an AAT defect and that patient exposed to or having a viral infection, by administering a compositions having AAT and/or peptides derived from the last 80 carboxyterminus of AAT to the AAT deficient patient.

Ninety-five patients with AAT deficiency replied who were receiving AAT replacement, and 46 AAT deficient individuals who were not taking AAT replacement replied (1 patient in each group possessed the mixed AAT phenotype SZ). The 95 patients receiving AAT replacement therapy were asked to compare the yearly incidence of lung infections before and after initiation of AAT therapy. Compared to the yearly number of lung infections reported prior to initiation of AAT therapy, a significant reduction in the number of lung infections was reported following the initiation of AAT therapy. Many patients also believed that head colds and flu were less frequent following the initiation of AAT replacement. In a separate comparison, the 95 members of the NHLBI cohort who received AAT replacement therapy were compared with the 46 who did not receive replacement therapy. The group receiving AAT therapy reported fewer yearly lung infections than did the group not receiving therapy.

Characteristics of the AAT-treated and non-treated groups were assessed for comparability in age, sex and smoking status. Taken together, the above results in vitro and in the NHLBI AAT-deficient registry subset suggest the possibility that AAT is a natural inhibitor of for example, Influenza A virus and CMV. Furthermore, investigation of AAT-deficient populations may provide a useful means of studying the association between AAT and infection with these viruses in vivo.

In one particular study, human subjects were assessed who have undergone lung transplantation. Since AAT-deficient patients often acquire severe emphysema which can require lung transplantation, these patients were studied epidemiologically. Following transplantation, the members of this study followed a strict protocol of medical management, and each receives frequent medical assessment. An extensive and detailed database is maintained on each of these patients. The database was inspected to evaluate, for example, the relationship between AAT deficiency and infection with Influenza A virus or with CMV. AAT deficient patients were found to have substantial and statistically significant increases in infection with influenza A (Flu) and with cytomegalovirus (CMV). These data establish AAT deficiency as one risk factor for infections with Flu and with CMV. Although this study was a correlation of AAT deficiency with increased occurrence of viral infection, further research was needed in order to establish a true relationship between AAT and viral infections.

### Cancer

In addition, embodiments herein concern compositions and methods of treatment to reduce or prevent viral-induced tumors by administering AAT or one or more peptides derived from the last 80 amino acids of the carboxyterminus of AAT. Non-limiting examples of virally-induced tumors include Rous sarcoma induced, human papilloma virus induced, polyoma induced, Hepatitis B virus induced and any other virally-induced tumor known in the art.

### Pneumonia

Pneumonia can be a common secondary infection from influenza. In certain embodiments, a therapeutically effective amount of AAT and/or one or more peptides derived from the last 80 amino acids of the carboxyterminus of naturally occurring AAT (SEQ ID NO: 20) can be administered to a subject having or exposed to viral pneumonia. Any composition disclosed herein may be used to prevent the onset or progression of pneumonia.

In certain embodiments, the reduction, prevention or inhibition of infection or side effects thereof associated with one or more of each of the above-recited conditions may be about 10-20%, 30-40%, 50-60%, or more reduction or inhibition due to administration of the disclosed compositions.

### Proteins and Polypeptides

One embodiment pertains to isolated proteins, and biologically active portions thereof, as well as polypeptide fragments, for example one or more peptides from the last 80 amino acids of naturally occurring AAT (SEQ ID NO:20). In some embodiments, the native polypeptide can be isolated from cells or tissue sources by a purification scheme using standard protein purification techniques known in the art. In certain embodiments, native polypeptides may be cleaved and peptides isolated to generate compositions of one or more peptides of the last 80 amino acids of the carboxyterminus of AAT. In other embodiments, compositions and methods may include one or more synthetic peptides designed to be one or more polpeptides of the last 80 amino acids of the carboxyterminus of AAT or any combination of peptides from that region disclosed herein. In certain embodiments, synthetic peptides may include, but are not limited to SEQ ID NOs. 1-19, SEQ ID NOs. 22-38 or any peptide contemplated herein. In another embodiment, polypeptides contemplated herein are produced by recombinant DNA techniques. Alternative to recombinant expression, a polypeptide can be synthesized chemically using standard peptide synthesis techniques.

Recombinant unmodified and mutant variants of AAT produced by genetic engineering methods are also known (U.S. Pat. No. 4,711,848). The nucleotide sequence of human AAT and other human AAT variants have been disclosed. In certain embodiments, nucleotide sequence or amino acid sequences from a mutant or variant form of AAT known in the art may be used as starting material to generate all of the AAT peptides contemplated herein if the variant also has the a conserved region of the last 80 amino acids of naturally occurring AAT, using recombinant DNA techniques and methods known to those of skill in the art.

An “isolated” or “purified” protein, peptide, or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium. When the protein is produced by chemical synthesis, it is preferably substantially free of chemical precursors or other chemicals. For example, such preparations of the protein have less than about 30%, 20%, 10%, 5% (by dry weight) of chemical precursors or compounds other than the polypeptide of interest.

Biologically active portions of a polypeptide can include polypeptides including amino acid sequences sufficiently identical to or derived from the amino acid sequence of the protein (e.g., the amino acid sequence shown in any of SEQ ID Nos: 1 to 19, 21-31 identified herein). A biologically active portion of a protein can be a polypeptide, which is, for example, 5, 10, 15, 20, 25, 30, 35, 40, 45, 50, 55, 60, 65, 70, 75 or 80 amino acids in length. Moreover, other biologically active portions, in which other regions of the protein are deleted, can be prepared by recombinant techniques and evaluated for one or more of the functional activities of the native form of a polypeptide of the invention.

In certain embodiments, polypeptides can include a polypeptide having an amino acid sequence of SEQ ID Nos: 1 to 19, 21-38 or other peptide derived from the last 80 amino acids of the carboxterminus of AAT identified herein. Other useful proteins are substantially identical (e.g., at least about 45%, preferably 55%, 65%, 75%, 80%, 85%, 90%, 95%, or 99%) to any of Nos: 1 to 19, 21-38 or other peptide derived from the last 80 amino acids of the carboxterminus of AAT identified herein, and retain the functional activity of the protein of the corresponding naturally-occurring protein yet differ in amino acid sequence due to derivation of peptide from the carboxyterminus or analogs thereof.

### Pharmaceutical Compositions

Compounds herein can be used as therapeutic agents in the treatment of a physiological (especially pathological) condition caused in whole or part, by a virus. In addition, a physiological (especially pathological) condition can be inhibited in whole or part. Peptides contemplated herein may be administered as free peptides or pharmaceutically acceptable salts thereof. Peptides may be administered to a subject as a pharmaceutical composition, which, can include the peptide and/or pharmaceutical salts thereof with a pharmaceutically acceptable carrier.

When utilizing BLAST, Gapped BLAST, and PSI-Blast programs, the default parameters of the respective programs (e.g., XBLAST and NBLAST) can be used.

Variants of the polypeptides are contemplated herein. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein may inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a composition of one or more peptides derived from the last 80 amino acids of the carboxyterminus of AAT compared to compostions of the naturally occurring form of AAT could have fewer side effects in a subject relative to treatment with the naturally occurring form of AAT.

### Fusion Polypeptides

In other embodiments, one or more peptides derived from the last 80 amino acids of the carboxyterminus of naturally occurring AAT (SEQ ID NO:20), may be part of a fusion polypeptide. In one example, a fusion polypeptide may include one or more of SEQ ID NOs: 1-19 and 21-31 or other disclosed peptides derived from naturally occurring AAT.

In yet other embodiments, a fusion polypeptide contemplated of use in methods herein can additionally include an amino acid sequence that is useful for identifying, tracking or purifying the fusion polypeptide, e.g., a FLAG or HIS tag sequence. The fusion polypeptide can include a proteolytic cleavage site that can remove the heterologous amino acid sequence from the compound capable of modulating onset of a viral infection and/or treating a viral infection contemplated herein

In one embodiment, fusion polypeptides can be produced by recombinant DNA techniques. Alternative to recombinant expression, a fusion polypeptide of the invention can be synthesized chemically using standard peptide synthesis techniques. In addition, a fusion polypeptide disclosed herein can include a pharmaceutically acceptable carrier, excipient or diluent.

In certain embodiments, a fusion protein can include a heterologous sequence derived from a member of the immunoglobulin protein family, for example, an immunoglobulin constant region, e.g., a human immunoglobulin constant region such as a human IgG1 constant region. A fusion protein can, for example, include one or more peptides derived from the last 80 amino acids of the carboxyterminus of AAT, or analog thereof fused with the amino-terminus or the carboxyl-terminus of an immunoglobulin constant region, by methods known in the art. In accordance with these embodiments, the FcR region of the immunoglobulin may be either wild-type or mutated. In certain embodiments, it is desirable to utilize an immunoglobulin fusion protein that does not interact with an Fc receptor and does not initiate ADCC reactions. In such instances, the immunoglobulin heterologous sequence of the fusion protein can be mutated to inhibit such reactions. See for example, U.S. Pat. No. 5,985,279 and WO 98/06248.

In yet another embodiment, AAT, analog thereof, polypeptide fusion protein can be a GST fusion protein in which is fused to the C-terminus of GST sequences. Fusion expression vectors and purification and detection means are known in the art.

Expression vectors can routinely be designed for expression of a fusion polypeptide disclosed herein in prokaryotic (e.g., E. coli, or eukaryotic cells (e.g., insect cells (using baculovirus expression vectors), yeast cells or mammalian cells) by any means known in the art. Expression of proteins in prokaryotes may be carried out by means known in the art.

In yet another embodiment, a nucleic acid of the invention can be expressed in mammalian cells using a mammalian expression vector as described in the art. In another embodiment, a recombinant mammalian expression vector is capable of directing expression of the nucleic acid in a particular cell type. A host cell can be any prokaryotic or eukaryotic cell. Vector DNA can be introduced into prokaryotic or eukaryotic cells via conventional transformation or transfection techniques.

### Other Agents

Any of the embodiments detailed herein may further include one or more a therapeutically effective amount of anti-microbial drugs, anti-inflammatory agent, immunomodulatory agent, or immunosuppressive agent or combination thereof.

Examples of anti-bacterial agents include, but are not limited to, penicillins, quinolonses, aminoglycosides, vancomycin, monobactams, cephalosporins, carbacephems, cephamycins, carbapenems, and monobactams and their various salts, acids, bases, and other derivatives.

Anti-fungal agents contemplated of use herein can include, but are not limited to, caspofungin, terbinafine hydrochloride, nystatin, amphotericin B, griseofulvin, ketoconazole, miconazole nitrate, flucytosine, fluconazole, itraconazole, clotrimazole, benzoic acid, salicylic acid, and selenium sulfide.

Anti-viral agents contemplated of use herein can include, but are not limited to, valgancyclovir, amantadine hydrochloride, rimantadin, acyclovir, famciclovir, foscamet, ganciclovir sodium, idoxuridine, ribavirin, sorivudine, trifluridine, valacyclovir, vidarabin, didanosine, stavudine, zalcitabine, zidovudine, interferon alpha, and edoxudine.

Immunomodulatory agents can include for example, agents which act on the immune system, directly or indirectly, by stimulating or suppressing a cellular activity of a cell in the immune system, (e.g., T-cells, B-cells, macrophages, or antigen presenting cells (APC)), or by acting upon components outside the immune system which, in turn, stimulate, suppress, or modulate the immune system (e.g., hormones, receptor agonists or antagonists, and neurotransmitters); other immunomodulatory agents can include immunosuppressants or immunostimulants. Anti-inflammatory agents can include, for example, agents which treat inflammatory responses, tissue reaction to injury, agents which treat the immune, vascular, or lymphatic systems or combination thereof.

Anti-inflammatory or immunomodulatory drugs or agents contemplated of use herein can include, but are not limited to, interferon derivatives, e.g., betaseron, β-interferon; prostane derivatives, iloprost, cicaprost; glucocorticoids such as cortisol, prednisolone, methylprednisolone, dexamethasone; immunsuppressive agents such as cyclosporine A, FK-506, methoxsalene, thalidomide, sulfasalazine, azathioprine, methotrexate; lipoxygenase inhibitors, e.g., zileutone, MK-886, WY-50295, SC-45662, SC-41661A, BI-L-357; leukotriene antagonists; peptide derivatives for example ACTH and analogs; soluble TNF (tumor necrosis factor)-receptors; TNF-antibodies; soluble receptors of interleukines, other cytokines, T-cell-proteins; antibodies against receptors of interleukins, other cytokines, and T-cell-proteins.

Other agents of use in combination with compositions described herein can be other molecules having serine protease inhibitor activity. For example serine protease inhibitors contemplated of use herein can include, but are not limited to, leukocyte elastase, thrombin, cathepsin G, chymotrypsin, plasminogen activators, and plasmin.

In certain embodiments, a composition may include one or more peptides derived from AAT where the peptide(s) may have similar activity to naturally occurring AAT. In each of the recited methods, one or more peptides derived from the last 80 amino acids of AAT contemplated for use within methods disclosed herein can include a series of peptides or analogs of these peptides. In certain embodiments, the peptides can be 5 or 10 or 15 or 20 or 25 or 30 or 35 or 40 or more amino acids in length. In certain embodiments, these peptides can include, but are not limited to, FVFLM (SEQ ID NO. 1), FVFAM (SEQ. ID NO. 2), FVALM (SEQ. ID NO. 3), FVFLA (SEQ. ID NO. 4), FLVFI (SEQ. ID NO. 5), FLMII (SEQ. ID NO. 6), FLFVL (SEQ. ID NO. 7), FLFVV (SEQ. ID NO. 8), FLFLI (SEQ. ID NO. 9), FLFFI (SEQ. ID NO. 10), FLMFI (SEQ. ID NO. 11), FMLLI (SEQ. ID NO. 12), FIIMI (SEQ. ID NO. 13), FLFCI (SEQ. ID NO. 14), FLFAV (SEQ. ID) NO. 15), FVYLI (SEQ. ID NO. 16), FAFLM (SEQ. ID NO. 17), AVFLM (SEQ. ID NO. 18), and any combination thereof.

In some embodiments, a composition comprising one or more pentapeptides may be used to modulate the onset or treat a subject exposed to, or having influenza. Influenza can be influenza A or B. In addition, influenza can be a subtype of influenza (e.g. H1N1).

In other embodiments, AAT peptides contemplated of use in the compositions and methods of the present invention are also intended to include any and all of those specific AAT peptides of SEQ ID NO. 20 depicted supra. Any combination of consecutive amino acids simulating AAT or AAT-like activity may be used, such as amino acids 315-324, 316-325, 317-326, 318-327, etc.

In each of the above-recited methods, α1-antitrypsin or analogs thereof are contemplated for use in a composition herein. These analogs may include peptides such as 10-mers, 15-mers, 20-mers etc. The peptides may include but are not limited to amino acid peptides containing GADLSGVTEE (SEQ ID NO:21); APLKLSKAVH (SEQ ID NO:22); KAVLTIDEKG (SEQ ID NO:22); TEAAGAMFLE (SEQ ID NO:23); RIPVSIPPEV (SEQ ID NO:24); KFNKPFVFLM (SEQ ID NO:25); IEQNTKSPLF (SEQ ID NO:26); MGKVVNPTQK (SEQ ID NO:27); LSGVTEEAPL (SEQ. ID NO. 28); KLSKAVHKAV (SEQ. ID NO. 29); LTIDEKGTEA (SEQ. ID NO. 30); AGAMFLERIP (SEQ. ID NO. 31); VSIPPEVKFN (SEQ. ID NO. 32); KPFVFLMIEQ (SEQ. ID NO. 33); NTKSPLFMGK (SEQ. ID NO. 34); VVNPTQK (SEQ. ID NO. 35); LEAIPMSIPPEVKFNKPFVFLM (SEQ ID NO: 36); and LEAIPMSIPPEVKFNKPFVF (SEQ ID NO: 37), SEQ ID NO:38 or any combination thereof.

In addition, other combination compositions of methods disclosed herein can include certain antibody-based therapies. Non-limiting examples include, polyclonal anti-lymphocyte antibodies, monoclonal antibodies directed at the T-cell antigen receptor complex (OKT3, TIOB9), monoclonal antibodies directed at additional cell surface antigens, including interleukin-2 receptor alpha. In certain embodiments, antibody-based therapies may be used as induction therapy in combination with the compositions and methods disclosed herein.

Subjects contemplated herein can include human subjects, or other subjects such as non-human subjects, including but not limited to, domesticated and non-domesticated animals and birds, for example, primates, dogs, cats, horses, llamas, sheep, birds, cows, pigs, guinea pigs, house birds, chickens and rodents.

### Pharmaceutical Compositions:

Embodiments herein provide for administration of compositions to subjects in a biologically compatible form suitable for pharmaceutical administration in vivo. By “biologically compatible form suitable for administration in vivo” is meant a form of the active agent (e.g. pharmaceutical chemical, protein, gene, antibody, or anti-viral agent) to be administered in which any toxic effects are outweighed by the therapeutic effects of the active agent. Administration of a therapeutically active amount of the therapeutic compositions is defined as an amount effective, at dosages and for periods of time necessary to achieve the desired result. For example, a therapeutically active amount of a compound may vary according to factors such as the disease state, age, sex, and weight of the individual, and the ability of antibody to elicit a desired response in the individual. Dosage regima may be adjusted to provide the optimum therapeutic response.

In one embodiment, a compound, agent or peptide composition (e.g. pharmaceutical chemical, protein, gene, antibody, or anti-viral agent) may be administered to a subject in need thereof subcutaneously, intravenously, by oral administration, inhalation, transdermally, intravaginally, topically, intranasally, rectally or a combination thereof. Depending on the route of administration, the active compound may be coated in a material to protect the compound from the degradation by enzymes, acids and other natural conditions that may inactivate the compound. In some embodiments, a compound or agent may be orally administered. In other embodiments, a compound or agent may be administered intravenously. In certain embodiments, the compound or agent may be administered intranasally, such as by inhalation.

A compound may be administered to a subject in an appropriate carrier or diluent, co-administered with enzyme inhibitors or in an appropriate carrier such as liposomes. The term “pharmaceutically acceptable carrier” as used herein is intended to include diluents such as saline and aqueous buffer solutions. It may be necessary to coat the compound with, or co-administer the compound with, a material to prevent its inactivation. The active agent may also be administered parenterally or intraperitoneally. Dispersions can also be prepared in glycerol, liquid polyethylene glycols, and mixtures thereof and in oils. Under some conditions of storage and use, these preparations may contain a preservative to prevent the growth of microorganisms.

Pharmaceutical compositions suitable for injectable use may be administered by means known in the art. For example, sterile aqueous solutions (where water soluble) or dispersions and sterile powders for the extemporaneous preparation of sterile injectable solutions or dispersion may be used. In all cases, the composition can be sterile and can be fluid to the extent that easy syringability exists. It might be stable under the conditions of manufacture and storage and may be preserved against the contaminating action of microorganisms such as bacteria and fungi. The pharmaceutically acceptable carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of microorganisms can be achieved by heating, exposing the agent to detergent, irradiation or adding various antibacterial or antifungal agents.

Sterile injectable solutions can be prepared by incorporating active compound (e.g. a compound capable of inhibiting viral infection) in an amount determined to be appropriate by a healthcare provider in a solvent with one or a combination of ingredients enumerated above, followed, for example, by filter sterilization.

Aqueous compositions can include an effective amount of a therapeutic compound, peptide, epitopic core region, stimulator, inhibitor, and the like, dissolved or dispersed in a pharmaceutically acceptable carrier or aqueous medium. Compounds and biological materials disclosed herein can be purified by means known in the art.

Solutions of the active compounds as free-base or pharmacologically acceptable salts can be prepared and suitably mixed with for example, a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms. Prolonged absorption of the injectable or ingestible compositions can be brought about by compositions of agents delaying absorption, for example, aluminum monostearate, gelatin or the like. In other embodiments, a composition contemplated herein can be in the form of a slow or time-released particle or capsule such as microparticles, for example, microbeads or a microgel. In accordance with these embodiments, a microparticle can contain a composition disclosed herein and once the microparticles are introduced to a subject in need of such a composition, the composition can be released upon targeting a specific region and/or upon introduction, in timed intervals or as the microparticles degrade. These methods are known in the art and are contemplated herein.

Therapeutic agents may be formulated within a mixture to include about 0.0001 to 1.0 milligrams, or about 0.001 to 0.1 milligrams, or about 0.1 to 1.0 or even about 1 to 10 gram per dose. Single dose or multiple doses can also be administered on an appropriate schedule for a predetermined condition.

In another embodiment, nasal solutions or sprays, aerosols or inhalants may be used to deliver the compound of interest. Additional formulations that are suitable for other modes of administration include suppositories and pessaries. A rectal pessary or suppository may also be used.

Oral formulations include such normally employed excipients as, for example, pharmaceutical grades of agents known in the art. In certain embodiments, oral pharmaceutical compositions can include an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations can contain at least 0.1% of active compound.

A pharmaceutical composition may be prepared with carriers that protect active ingredients against rapid elimination from the body, such as time-release formulations or coatings. Such carriers include controlled release formulations, such as, but not limited to, microencapsulated delivery systems, and biodegradable, biocompatible polymers, such as ethylene vinyl acetate, polyanhydrides, polyglycolic acid, polyorthoesters, polylactic acid and others are known.

Pharmaceutical compositions are administered in an amount, and with a frequency, that is effective to inhibit or alleviate side effects of a transplant and/or to reduce or prevent rejection. The precise dosage and duration of treatment may be determined empirically using known testing protocols or by testing the compositions in model systems known in the art and extrapolating therefrom. Dosages may also vary with the severity of the condition. A pharmaceutical composition is generally formulated and administered to exert a therapeutically useful effect while minimizing undesirable side effects. In general, an oral dose ranges from about 200 mg to about 1000 mg, which may be administered for example, 1 to 3 times per day.

It is contemplated that, for a particular subject, specific dosage regimens may be adjusted over time according to need. A preferred dose for administration can be anywhere in a range between about 0.01 mg and about 100 mg per ml of biologic fluid of treated subject. In one embodiment, a range can be between 1 and 100 mg/kg which can be administered daily, every other day, biweekly, weekly, monthly etc. In another particular embodiment, the range can be between 10 and 75 mg/kg introduced weekly to a subject. A therapeutically effective amount of AAT, peptides, or drugs that have similar activities as AAT or peptides can be also measured in molar concentrations and can range between about 1 nM to about 2 mM. It is understood in the art that doses introduced by inhalation may be considerably lower than doses introduced to a subject by other routes.

Compositions herein may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent.

Liposomes can be used as a therapeutic delivery system and can be prepared in accordance with known laboratory techniques. In addition, dried lipids or lyophilized liposomes prepared as previously described may be reconstituted in a solution of active agent (e.g. nucleic acid, peptide, protein or chemical agent), and the solution diluted to an appropriate concentration with a suitable solvent. The amount of active agent encapsulated can be determined in accordance with standard methods.

In a one embodiment, a nucleic acid (e.g. AAT or nucleic acid sequences that code for one or more peptides derived from the last 80 amino acids of the carboxyterminus of AAT) and the lipid dioleoylphosphatidylcholine may be employed. For example, nuclease-resistant oligonucleotides may be mixed with lipids in the presence of excess t-butanol to generate liposomal-oligonucleotides for administration.

Pharmaceutical compositions containing AAT, or one or more peptides derived from AAT may be administered to a subject, for example by subcutaneously, intramuscularly, intranasally, orally, topically, transdermally, parenterally, gastrointestinally, transbronchially and transalveolarly. Topical administration is accomplished via a topically applied cream, gel, rinse, etc. containing therapeutically effective amounts of inhibitors of serine proteases. Transdermal administration can be accomplished by application of a cream, rinse, gel, etc. capable of allowing compositions described to penetrate the skin and enter the blood stream. In addition, osmotic pumps may be used for administration. The necessary dosage will vary with the particular condition being treated, method of administration and rate of clearance of the composition from the body.

In each of the aforementioned compositions and methods, a compositions may be used in a single therapeutic dose, acute manner or a chronic manner to treat episodes or prolonged bouts, respectively, in reducing or eliminating a viral-associated disorder contemplated herein.

### AAT

Naturally occurring AAT/α1-antitrypsin is a glycoprotein having 394 amino acids. Human AAT is a single polypeptide chain and can be represented by SEQ ID NO:20.

Extrahepatic sites of AAT production include neutrophils, monocytes and macrophages, and the expression of AAT is inducible in response to LPS, TNFα, IL-1 and IL-6 in various cell types. Deficiency in AAT can be associated with immune dysfunctional conditions such as rheumatoid arthritis and systemic lupus erythematosus.

Serine protease inhibitor molecules, which may be used in combination with compositions disclosed herein may include compounds disclosed in the following: WO 98/20034 disclosing serine protease inhibitors from fleas; WO98/23565 disclosing aminoguanidine and alkoxyguanidine compounds useful for inhibiting serine proteases; WO98/50342 disclosing bis-aminomethylcarbonyl compounds useful for treating cysteine and serine protease disorders; WO98/50420 cyclic and other amino acid derivatives useful for thrombin-related diseases; WO 97/21690 disclosing D-amino acid containing derivatives; WO 97/10231 disclosing ketomethylene group-containing inhibitors of serine and cysteine proteases; WO 97/03679 disclosing phosphorous containing inhibitors of serine and cysteine proteases; WO 98/21186 benzothiazo and related heterocyclic inhibitors of serine proteases; WO 98/22619 disclosing a combination of inhibitors binding to P site of serine proteases with chelating site of divalent cations; WO 98/22098 disclosing a composition which inhibits conversion of pro-enzyme CPP32 subfamily including caspase 3 (CPP32/Yama/Apopain); WO 97/48706 disclosing pyrrolo-pyrazine-diones; and WO 97/33996 disclosing human placental bikunin (recombinant) as serine protease inhibitor.

### Kits

Other embodiments concern kits for use with compositions and methods described above. In certain embodiments, small molecules, proteins or peptides may be employed for use in any of the disclosed methods. In addition, other agents such as anti-bacterial agents, immunosuppressive agents, anti-inflammatory agents, and/or anti-viral agents may be provided in the kit. The kits can include, a suitable container (e.g. vial, syringe, bottle, tube etc.) a protein or a peptide or analog agent, and optionally one or more additional agents.

Kits may further include a suitably aliquoted composition of the encoded protein or polypeptide antigen, whether labeled or unlabeled, as may be used to prepare a standard curve for a detection assay. In certain embodiments, a kit may include a composition including, but not limited to, AAT or an AAT analog or polypeptide having no significant serine protease inhibitor activity or a peptide or combination of peptides derived from the last 80 amino acids of the carboxy terminus of SEQ ID NO:20.

A container of kits contemplated herein will generally include at least one vial, test tube, flask, bottle, syringe or other container means, into which an agent or agents may be placed, and preferably, suitably aliquoted. In accordance with these embodiments, a kit can contain compositions of AAT or one or more peptides derived from the last 80 amino acids of the carboxyterminus of SEQ ID NO:20 or other agents contemplated herein (e.g. other anti-viral agents). Such containers may include injection or blow-molded plastic containers into which the desired vials are retained.

### EXAMPLES

The following examples are included to illustrate various embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes may be made in the specific embodiments which are disclosed and still obtain a like or similar result without departing from the spirit and scope of the invention.

### General Procedure and Materials

In one exemplary method, AAT used in these studies is purified from the blood of healthy volunteers. AAT is purified to single-band homogeneity. The AAT protein is diafiltered into a diluent consisting of NaCl, sodium phosphate, pH 7.05. The AAT preparations are maintained at stock concentrations of 14-50 mg/ml and stored at −70.degree. C. until added to cultures.

### U1 Cells

Medium for monocytic U1 cell and MAGI-CCR5 cell cultures consists of RPMI 1640 medium purchased from Mediatech (Hermdon, Va.) containing 2.5 mM L-glutamine, 25 mM Hepes, 100 units/ml penicillin and streptomycin (GIBCO/BRL, Rockville, Md.) with 10% or 7.5% (vol/vol) heat-inactivated fetal bovine serum (FBS, GIBCO) for U1 cell and MAGI-CCR5 cell cultures, respectively. PBMC are cultured in R3 medium consisting of RPMI 1640 medium (Mediatech), 20% FBS (GIBCO), 100 units/ml penicillin and streptomycin (GIBCO) and 5% (vol/vol) IL-2 (Hemagen, Waltham, Mass.).

U1 monocytic cell assay. U1 cells can be obtained from the AIDS Research and Reference Reagent Program, National Institute of Allergy and Infectious Diseases, NIH. U1 cells are maintained in T-175 polystyrene flasks (Falcon, Becton Dickinson, Franklin Lakes, N.J.) in medium and used when in log phase growth. Cells are counted in a hemacytometer, examined for viability by Trypan blue exclusion (>95% for all experiments) and resuspended in fresh medium at 2×10⁶ per ml. Two-hundred fifty ml of cell suspension are added to wells of 24-well polystyrene tissue culture plates (Falcon), followed by the addition of medium or AAT to produce the final concentration to be tested in a volume of 450 ml. After 1.0 hr of incubation (37° C., 5% CO₂), 50 ml of medium (control) or stimulus diluted in medium are added to wells to produce the final concentration of stimulus to be tested. The final culture volumes are 500 ml and contained 1×10⁶ cells per ml. After 48 hr of incubation (37° C., 5% CO₂) 50 ml of 10% (vol/vol) Triton-X-100 is added to each culture (final concentration of 1% vol/vol), and cultures are frozen and thawed once. This is followed by assay for HIV p24 antigen by ELISA with a lower limit of detection of 31 pg/ml (NCl-Frederick Cancer Research and Development Center, Frederick, Md.). The disruption of cells due to the addition of Triton-X-100 and the freeze-thaw cycle produced cell lysates and enabled assessment of total (secreted and cell-associated) production of p24 antigen.

### Example 1

### General Procedure and Materials

Alpha-1-antitrypsin (AAT) used in these studies is purified from the blood of healthy volunteers. AAT is purified to single-band homogeneity. The AAT protein is diafiltered into a diluent consisting of NaCl, sodium phosphate, pH 7.05. The AAT preparations are maintained at stock concentrations of 14-50 mg/ml and stored at −70.degree. C. until added to cultures. As a control AAT preparation that is different from the composition of the invention a commercially available Prolastin (Bayer's AAT) is used. Recombinant human interleukin (IL)-18 is obtained from Vertex Pharmaceuticals Inc., (Cambridge, Mass.). IL-6 and tumor necrosis factor (TNF) are obtained from R & D Systems, Minneapolis, Minn., endotoxin-free NaCl, and endotoxin (lipopolysaccharide, LPS) is obtained from Sigma (St. Louis, Mo.).

Medium for monocytic U1 cell and MAGI-CCR5 cell cultures consists of RPMI 1640 medium purchased from Mediatech (Hermdon, Va.) containing 2.5 mM L-glutamine, 25 mM Hepes, 100 units/ml penicillin and streptomycin (GIBCO/BRL, Rockville, Md.) with 10% or 7.5% (vol/vol) heat-inactivated fetal bovine serum (FBS, GIBCO) for U1 cell and MAGI-CCR5 cell cultures, respectively. PBMC are cultured in R3 medium consisting of RPMI 1640 medium (Mediatech), 20% FBS (GIBCO), 100 units/ml penicillin and streptomycin (GIBCO) and 5% (vol/vol) IL-2 (Hemagen, Waltham, Mass.).

U1 monocytic cell assay. U1 cells are obtained from the AIDS Research and Reference Reagent Program, National Institute of Allergy and Infectious Diseases, NIH. U1 cells are maintained in T-175 polystyrene flasks (Falcon, Becton Dickinson, Franklin Lakes, N.J.) in medium and used when in log phase growth. Cells are counted in a hemacytometer, examined for viability by Trypan blue exclusion (>95% for all experiments) and resuspended in fresh medium at 2.times.10.sup.6 per ml. Two-hundred fifty ml of cell suspension are added to wells of 24-well polystyrene tissue culture plates (Falcon), followed by the addition of medium or AAT to produce the final concentration to be tested in a volume of 450 ml. After 1.0 hr of incubation (37° C., 5% CO.sub.2), 50 ml of medium (control) or stimulus diluted in medium are added to wells to produce the final concentration of stimulus to be tested. The final culture volumes are 500 ml and contained 1.times.10.sup.6 cells per ml. After 48 hr of incubation (37° C. and 5% CO.sub.2) 50 ml of 10% (vol/vol) Triton-X-100 (Fisher Scientific, Fair Lawn, N.J.) is added to each culture (final concentration of 1% vol/vol), and cultures are frozen and thawed once. This is followed by assay for HIV-1 p24 antigen by ELISA with a lower limit of detection of 31 pg/ml (NCl-Frederick Cancer Research and Development Center, Frederick, Md.). The disruption of cells due to the addition of Triton-X-100 and the freeze-thaw cycle produced cell lysates and enabled assessment of total (secreted and cell-associated) production of p24 antigen.

### Peripheral Blood Mononuclear Cells (PBMC) Based HIV Assay.

These studies are approved by the Combined Investigation Review Board of the University of Colorado Health Sciences Center. PBMC from HIV-1 negative healthy subjects are isolated from heparinized blood by Ficoll-Hypaque density-gradient centrifugation. The concentration of PBMC in aliquots are counted using a hemacytometer (viability>95% by trypan blue exclusion for each experiment) and PBMC are diluted at 1.times.10.sup.6 per ml in R3 medium supplemented with additional 5% (vol/vol) IL-2 and 3.3 mg/ml phytohemagglutinin (PHA, Sigma). Cell suspensions are then incubated for 2 days (37° C., 5% CO.sub.2) in T-175 polystyrene tissue culture flasks (Falcon).

The stocks of lymphocyte-tropic HIV-1 strain A018A are titered by standard protocol and are used to infect PBMC. Following the 2 days of incubation, PBMC from each donor are removed from tissue culture flasks, divided into 2 equal aliquots placed into 50 ml polypropylene tubes (Falcon), concentrated by centrifugation and the medium decanted. Each parallel aliquot is infected by incubation with 300 tissue culture infective doses (TCID).sub.50HIV-1 per 1.times.10.sup.6 cells for 3 hr in 500 ml medium. The parallel PBMC infections from each donor are conducted in the absence or presence of 3 mg/ml AAT. The infected PBMC (without or with 3.0 mg/ml AAT) are then resuspended and washed in 15 ml R3 medium, pelleted, and resuspended at 2.times.10.sup.6 per ml in fresh R3 medium. Two hundred fifty ml of HIV-1-infected PBMC is aliquoted into 24-well polystyrene tissue culture plates (Falcon). An additional 250 ml R3 medium (control) or AAT is added to appropriate wells to produce a final culture volume of 500 ml containing 1.times.10.sup.6 cells per ml. For each donor, a separate 250 ml aliquot of PBMC suspension is added to a 1.5 ml polypropylene microfuge tube (Fisher) along with 200 ml R3 medium and 50 ml of 10% (vol/vol) Triton-X-100 (Fisher). This sample is frozen and designated time 0. Cultures in 24-well plates are incubated for 4 days, after which Triton-X-100 (Fisher) is added (final concentration of 1% vol/vol as described above for U1 cell cultures) and plates frozen and thawed once. Corresponding time 0 samples are thawed with each plate and cell lysates assayed for p24 antigen by ELISA.

### MAGI-CCRS Cell Assay.

The MAGI (Multinuclear Activation of a Galactosidase Indicator)-CCR-5 cell line is a clone derived from the HeLa cell line that expresses high levels of CD4. It has been transfected with a single integrated copy of a galactosidase gene under control of the HIV-1 long terminal repeat. Beta-galactosidase is expressed upon production of HIV-1 Tat protein following one round of HIV-1 replication within the cell. The MAGI-CCR-5 cell line is derived from MAGI cells into which the CCR-5 HIV-1 co-receptor gene has been incorporated. These cells constitute an assay for early infection events and can be infected with either lymphocyte-tropic or macrophage-tropic HIV-1 strains. MAGI-CCR-5 cells are obtained from the AIDS Research and Reference Reagent Program, National Institute of Allergy and Infectious Diseases, NIH. Cells are cultured in polystyrene T-175 flasks (Falcon) in medium until cells are noted to be in log growth phase. Cells are then resuspended in fresh medium and aliquoted into 24-well polystyrene plates (Falcon) at 4.times.10.sup.4 cells per well (1 ml total volume). After 24 hr incubation adherent cells are 30-40% confluent and all medium is removed. Two hundred ml of fresh medium is then added to each well without (negative control) or with AAT and incubated for 1 hour. AAT diluent is added to a separate well at a volume equivalent to that of the highest concentration of AAT tested (control).

One hundred thirty TCID.sub.50 of HIV-1 and DEAE dextran in medium are added to each well. T-cell tropic HIV-1 strain A018A is used. After 2 hr incubation, medium is added to each well to adjust the final volume of each well to 500 ml. Cultures are incubated for 48 hr, which allows infection of the MAGI-CCR-5 cells. Medium is aspirated and the cells fixed for 5.0 min at room temperature by adding 1.0 ml of a 1% formaldehyde/0.2% glutaraldehyde solution in phosphate buffered saline (PBS). Fixing solution is then aspirated and cells washed with PBS. This is followed by addition of galactosidase staining solution. Fifty min of incubation is followed by a blinded optical count of pigmented cells under a microscope.

### Statistical Analysis.

Data are presented as means.+−.SEM. Group means are compared by ANOVA using Fisher's least significant difference. For data expressed as percent change, the values for p24 in control cultures (medium alone) are subtracted from those for each culture-containing stimulus. The p24 concentrations in cultures conducted in the presence of stimulus alone are set at 100%. Percent p24 in cultures containing stimulus and AAT are calculated by dividing the measured p24 by that present in cultures containing stimulus alone. The resultant fraction is expressed as a percent.

### Example 2

### Anti-HIV Effect of AAT

AAT Inhibits Production of HIV-1 in U1 Cell Cultures. The U1 cell line is derived from human monocytic U937 cells into which 2 copies of HIV-1 provirus are incorporated into host genome. Exposing U1 cells to pro-inflammatory cytokines such as IL-18, IL-1, IL-6 and TNF, phorbol esters or hyperosmolarity results in the induction of HIV-1 as assessed by p24 antigen. Stimulation of U1 cells with 0.5 nM IL-18 induced large amounts of p24 antigen after 48 hr of incubation in 3 separate experiments. U1 cells cultured in medium alone (control) contained a mean of 41.3.+−.11.5 pg/ml p24 antigen, which is increased 150-fold to 6,235.+−.1,775 pg/ml p24 following stimulation with IL-18. Cultures conducted in the presence of AAT added 1 hour prior to the addition of IL-18 demonstrated a dose-dependent reduction in p24, with near ablation of IL-18-induced p24 observed at 3 mg/ml AAT. AAT added at 0.1, 0.5, 1, 2 and 3 mg/ml resulted in 6,879.+−.207, 3,687.+−.968, 2,029.+−.625, 452.+−.209 and 179.+−.79 pg/ml p24 production, respectively. At 1, 2 and 3 mg/ml AAT, the percent reductions observed compared to stimulation with IL-18 alone are 65.+−.1.8, 93.+-.3.0 and 98.+−.1%, respectively.

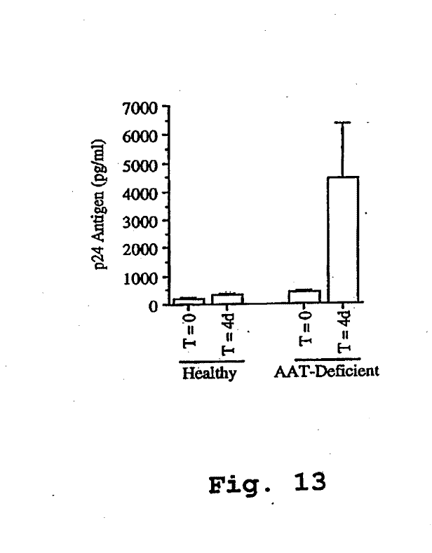

To evaluate the effect of AAT on U1 cell proliferation and viability, 3 experiments are performed in the presence or absence of 5 mg/ml AAT. U1 cells are added at 1.times.10.sup.6 cells per ml and cultured for 48 hrs. Following incubation, cells are quantified using a hemacytometer. The mean.+−.SEM cell concentrations in control and AAT-containing cultures are 2.5.times.10.sup.6.+−.0.5.times.10.sup.6 and 2.4.times.10.sup.6.+−.0.3.times.10.sup.6 respectively. These values are each significantly higher than the 1.times.10.sup.6 cells per ml added initially (P<0.05), but they are not significantly different from one another. For all cultures, cell viability by trypan blue exclusion is >95%. The lack of toxicity is illustrated in FIG. 13.

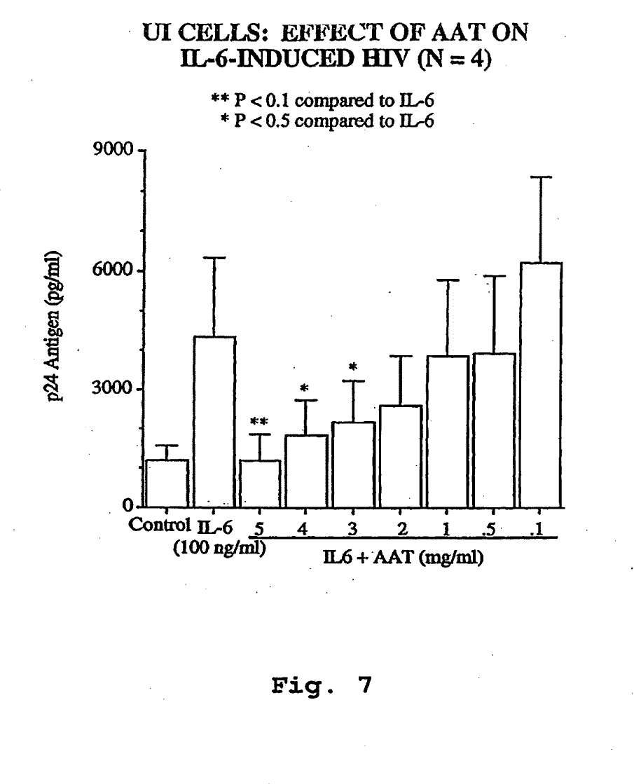

In 4 separate experiments, using 100 ng/ml IL-6 as a stimulus, the mean p24 antigen measured in U1 cells cultured in medium alone (control) is 1,207.+−.361 pg/ml (FIG. 7). Stimulation with 100 ng/ml IL-6 results in a 3.6-fold increase in p24 antigen production, to 4,337.+−.2,006 pg/ml. Stimulation with IL-6 in the presence of AAT results in dose-dependent inhibition of p24 production compared to that measured in the absence of AAT. With the addition of AAT at 0.1, 0.5, 1, 2, 3, 4, and 5 mg/ml, the measured P24 antigen values are 6,228.+−.2,129, 3,992.+−.1,987, 3,850.+−.1,943, 2,597.+−.1,253, 2,155.+−.1,085, 1,838.+−.881 and 1,213.+−.668 pg/ml, respectively. The corresponding mean percent reductions for AAT additions of 3, 4 and 5 mg/ml are 80, 88 and 100%, respectively.

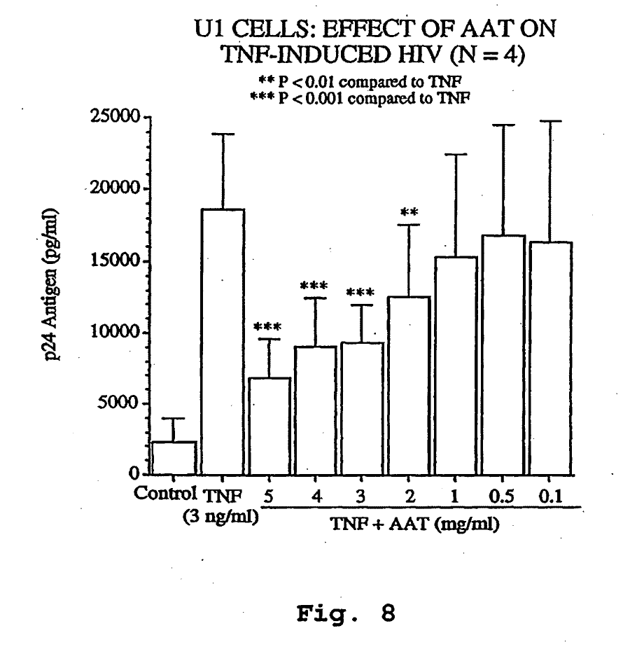

In 4 separate experiments, obtained in U1 cells exposed to TNF as stimulus, the mean p24 antigen measured in control and TNF-stimulated (3.0 ng/ml) cultures are 2,328.+−.1,680 and 18,635.+−.5,243 pg/ml, respectively (FIG. 8). This 8-fold increase in p24 production is significantly and dose-dependently reduced in the presence of AAT. Inclusion of AAT at the concentrations 0.1, 0.5, 1, 2, 3, 4, and 5 mg/ml reduced TNF-induced p24 antigen to 16,405.+−.8,449, 16,863.+−.7,718, 15,328.+−.7,129, 12,566.+−.4,981, 9,341.+−.2,730, 9,091.+−.3,436 and 6,868.+−.2,737, respectively. The mean percent reductions in TNF-induced p24 antigen observed in the presence of 3, 4, and 5 mg/ml AAT are 56, 60, and 73%, respectively.

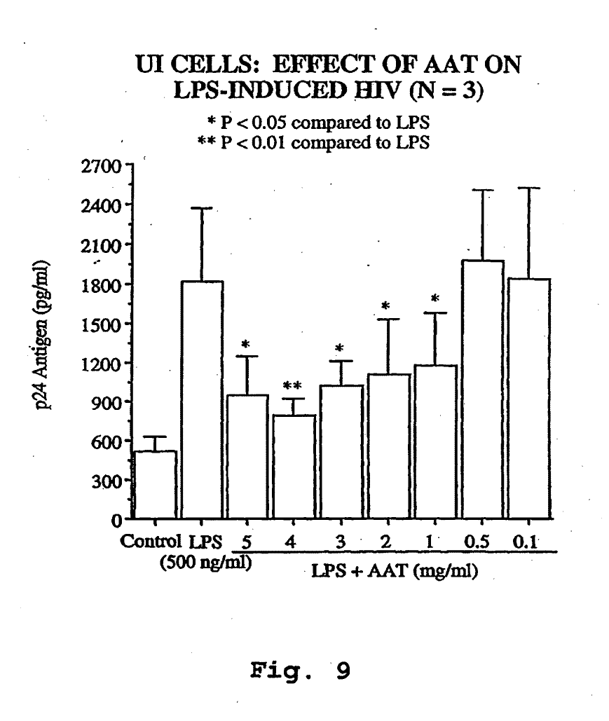

LPS is a cell wall component of gram-negative bacteria with several pro-inflammatory activities. In 3 experiments, U1 cells cultured in the presence of 500 ng/ml LPS for 48 hrs contained 1,427.+−.39 p24 antigen, as shown in FIG. 9. This represents a mean 3-fold increase compared to p24 produced in control (medium alone) cultures, where 476.+−.76 pg/ml p24 antigen was measured. U1 cells stimulated with LPS in the presence of 0.1, 0.5, 1, 2, 3, 4, and 5 mg/ml AAT contained 1,531,.+−.436, 1,543,.+−.427, 1,108.+−.241, 913.+−.287, 782,.+−.187, 578,.+−.155, 626.+−.257, pg/ml p24 antigen, respectively. Addition of AAT at 3, 4, and 5 mg/ml inhibited p24 production by 71, 90 and 86%, respectively.

### AAT Inhibits NaCl-Induced HIV-1 in U1 Cell Cultures.

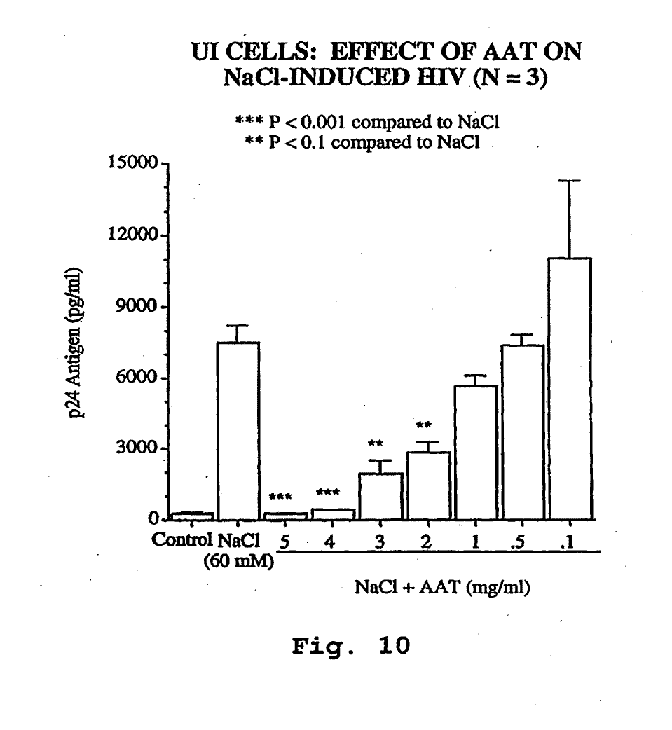

To exclude the possibility that AAT-induced inhibition of cytokine-stimulated p24 is due to protein-protein interactions, hyperosmolarity is used as the p24-inducing stimulus. Previous studies have established 60 mM NaCl as a potent inducer of p24 antigen in U1 cell cultures. The effect of AAT on NaCl-induced p24 in 3 experiments is tested and the results are shown in FIG. 10. A large (26-fold) increase in mean p24 antigen production in cultures is observed in the presence of NaCl alone as compared to control (medium alone) cultures. The mean p24 antigen measured in NaCl-stimulated and control cultures are 7,511.+−.707 and 295.+−.29 pg/ml, respectively. Stimulation with 60 mM NaCl in the presence of 0.1, 0.5, 1.0, 2.0, 3.0, 4.0 and 5.0 mg/ml AAT resulted in mean p24 levels of 11,054.+−.3,231, 7,363.+−.485, 5,657.+−.48, 2,83 8.+−.466, 1,919.+−.594, 425.+−.32 and 266.+−.26 pg/ml, respectively. For AAT added at 3.0, 4.0 and 5.0 mg/ml the corresponding percent inhibitions are 76, 98.3 and 100% (FIG. 10).

### AAT Inhibits p24 Antigen Production in HIV-1-Infected PBMC.

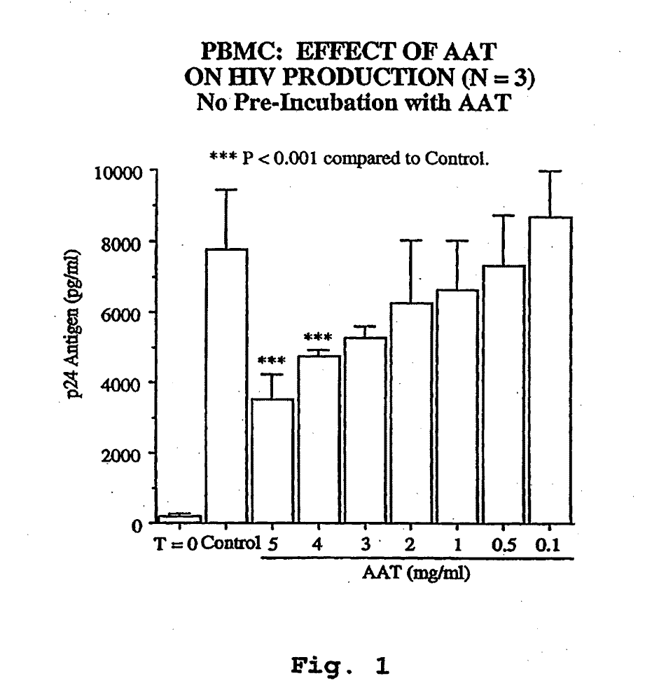

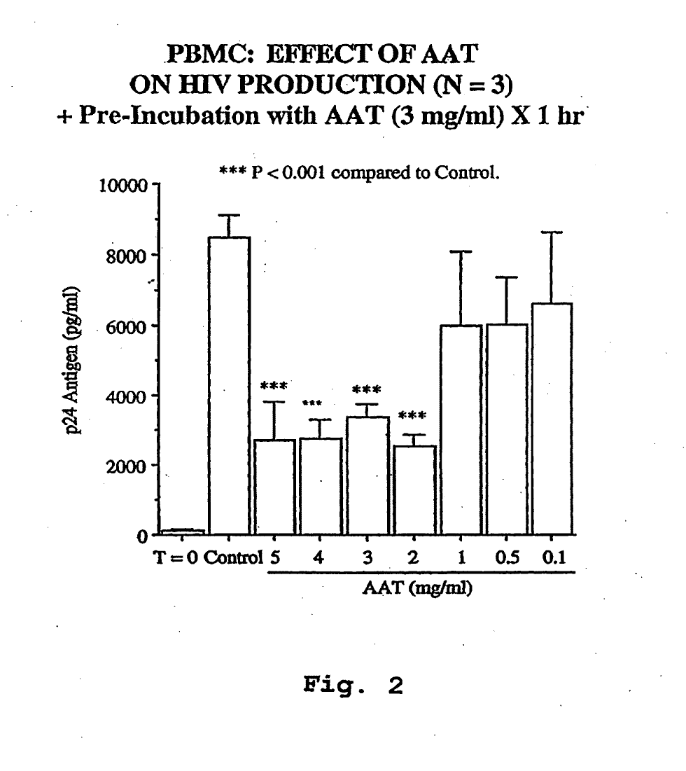

The effect of AAT on freshly-infected PBMC is tested to assess activity in a primary cell model of HIV-1 infection. PBMC isolated from 3 healthy volunteers are infected with lymphocyte-tropic HIV-1 as described above. FIGS. 1 and 2 show results obtained for PBMC infected with HIV-1 in the absence or presence of 3 mg/ml AAT at the time of infection. A large increase in p24 antigen occurred over the 4 days of culture, with 180.+−.63 pg/ml p24 measured at time t=0 and 7,781.+−.1,650 pg/ml p24 measured after 4 days (R3 medium alone, control). This represents a mean 43-fold increase in p24 (P<0.001). Under these conditions, PBMC cultured for 4 days with AAT added at 0.1, 0.5, 1.0, 2.0, 3.0, 4.0 and 5.0 mg/ml produced 8,687.+−.1,304, 7,392.+−.1,299, 6,613, 6,258.+−.1,772, 5,275.+−.316,4,725.+−.101, and 3,508 pg/ml p24, respectively. Compared to control cultures, significant reductions in p24 antigen are observed for added AAT concentrations of 4.0 and 5.0 mg/ml (22 and 46% reductions, respectively).

As shown in (b), compared to time 0 a significant increase in p24 production is observed in control cultures after 4 days of culture, with values of 107.+−.52 and 8,478.+−.629 pg/ml, respectively (mean 79-fold increase, P<0.001). PBMC cultured in the presence of AAT added at 0.1, 0.5, 1.0, 2.0, 3.0, 4.0 and 5.0 mg/ml produced 6,620.+−.2,026, 6,047.+−.1,322, 6,014.+−.2,055, 2,516.+−.345, 2,743.+−.316 and 2,713.+−.645 pg/ml, respectively. Significant reductions in p24 antigen in cultures exposed to AAT compared to control cultures are observed for AAT concentrations of 2.0, 3.0, 4.0 and 5.0 mg/ml AAT. Compared to control cultures, these AAT concentrations resulted in reductions in p24 production of 71, 61, 65 and 67%, respectively.

### AAT Inhibits Early Infection-Associated Events in MAGI-CCR5Cells Exposed to HIV-1.

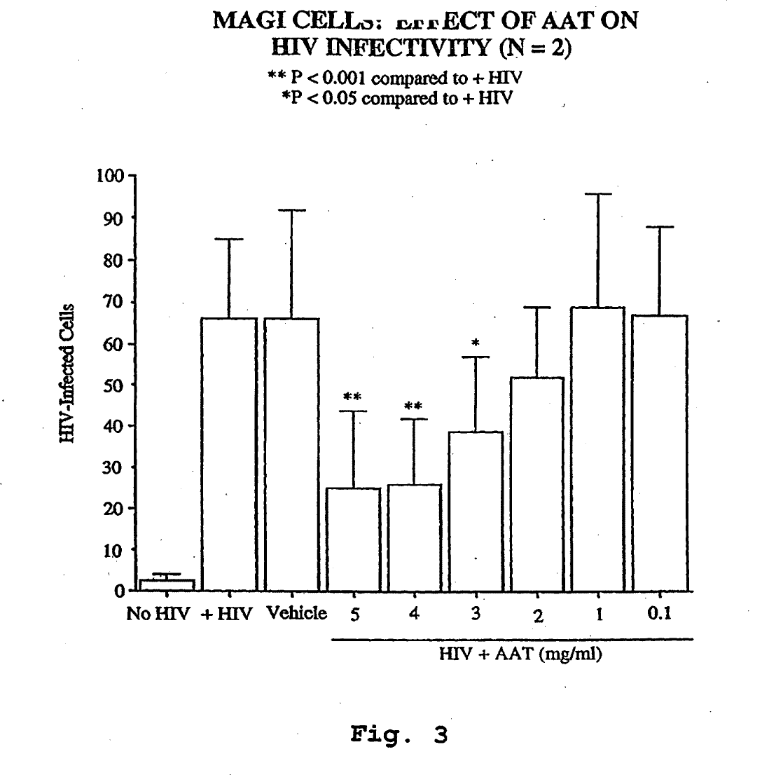

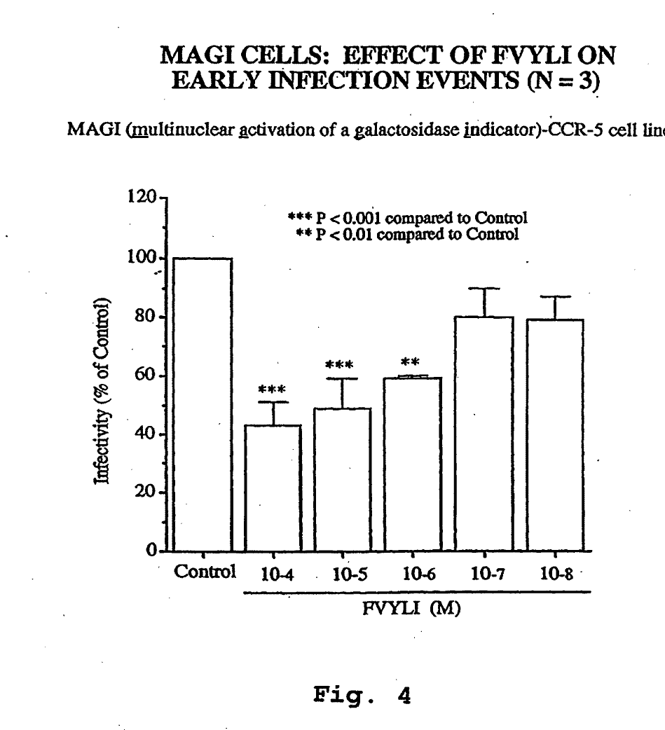

The MAGI-CCR-5 cell assay evaluates early events in the HIV-1 infection process. These events include cell-surface binding and internalization, uncoating, reverse transcription and translation, protein processing and Tat activity. Binding of the tat protein to a reporter construct within the MAGI-CCR-5 cells enables quantification of these early HIV-1 events. In 3 separate experiments shown in FIGS. 3 and 4, MAGI-CCR-5 cells are infected with A018A strain of HIV-1 as described supra. In cultures conducted in the absence of virus (no HIV-1), a mean positive cell count of 2.3 is obtained. In the presence of HIV-1 (+HIV-1), an increase in mean positive cell count is observed, to 72.+−.13 (31-fold increase, P<0.001). MAGI-CCR-5 cells exposed to HIV-1 and cultured with added AAT demonstrate significant and dose-dependent inhibition of positive cell counts. Addition of 0.1, 1.0, 2.0, 3.0, 4.0 and 5.0 mg/ml AAT resulted in mean positive cell counts of 74.+−.13, 75.+−.17, 56.+-.II, 45.+−.12, 28.+−.9, and 21.+−.12, respectively.

Compared to cultures containing HIV-1 alone, significant inhibition of MAGI-CCR-5 cell early infection events is significant for AAT concentrations of 2.0, 3.0, 4.0 and 5.0 mg/ml. These values correspond to 23, 41, 66 and 76% inhibition. As a vehicle control, MAGI-CCR-5 cells are exposed to virus and a diluent volume equivalent to that of AAT solution added to 5.0 mg/ml cultures. Cultures containing diluent produced a positive cell count of 72.+−.16, which is not significantly different from cultures containing HIV-1 alone (+HIV), as shown on the horizontal axis.

### Example 3

### Failure of Commercial AAT Preparation (Prolastin) to Inhibit HIV

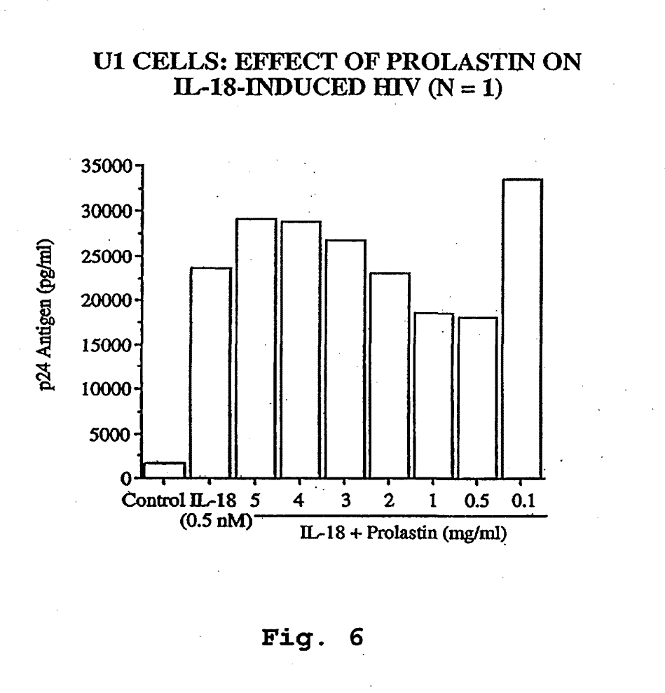

Prolastin used as a control preparation of AAT in the experimental setting that is similar to those described above. Surprisingly, this preparation fails to display anti-HIV activity at doses that are comparable to the composition of the invention (FIG. 6). The lack of the activity cannot be explained by low levels of active AAT since Prolastin contains only about 8% of inactive form of total antitrypsin (Lomas D A, Elliott P R, Carrell R W. Commercial plasma alpha1-antitrypsin (Prolastin) contains a conformationally inactive, latent component. Eur Respir J March 1997; 10(3):672-5). The biological significance of this observation is unknown. However, this means that not every AAT composition is inherently antivirally active, which may explain why prior to this invention others failed to discover the anti-HIV activity of AAT. Upon this unexpected observation a series of tests are carried out to further investigate the significance of AAT and its role as naturally occurring anti-HIV substance. Whole blood collected from at least 12 healthy donors and containing relatively normal levels of functionally active AAT is resistant to HIV infection. As can be seen from FIG. 13, in healthy individuals HIV p24 antigen levels on day 4 postinfection (T=4d) are not significantly higher than at inoculation (T=0) (shown in FIG. 13 as two bars on the left). In contrast, blood from AAT-deficient humans is highly susceptible to HIV infection. FIG. 13 shows that lack of functional AAT makes cells from such individuals prone to HIV infection.

### Example 4

### Effect of Select Peptides on HIV

FIG. 4 shows representative results obtained with a carboxyterminal peptide FVYLI (SEQUENCE ID NO. 16) that is derived but not necessarily identical to a respective C-terminal pentapeptide from AAT. Other short peptides such as FVFLM (SEQUENCE ID NO. 1), FVFAM (SEQUENCE ID NO. 2), FVALM (SEQUENCE ID NO. 3), FVFLA (SEQUENCE ID NO. 4), FLVFI (SEQUENCE ID NO. 5), FLMII (SEQUENCE ID NO. 6), FLFVL (SEQUENCE ID NO. 7), FLFVV (SEQUENCE ID NO. 8), FLFLI (SEQUENCE ID NO. 9); FLFFI (SEQUENCE ID NO. 10), FLMFI (SEQUENCE ID NO. 11), FMLLI (SEQUENCE ID NO. 12), FIIMI (SEQUENCE ID NO. 13), FLFCI (SEQUENCE ID NO. 14), FLFAV (SEQUENCE ID NO. 15), FVYLI. (SEQUENCE ID NO. 16), FAFLM (SEQUENCE ID NO. 17), AVFLM (SEQUENCE ID NO. 18) demonstrate more or less similar effect (not shown). They are active at approximately similar molar range when used alone or in combination, when mixtures thereof are added to the MAGI cultures. It is concluded that peptides derived from or homologous and/or analogous to this particular C-terminal region of AAT are equally antivirally active as a whole AAT molecule. This observation is totally unexpected since peptide fragments of such size are not anticipated to replace large size AAT molecule.

### Example 5

### Anti-HIV Effect of Drugs Having AAT Activity

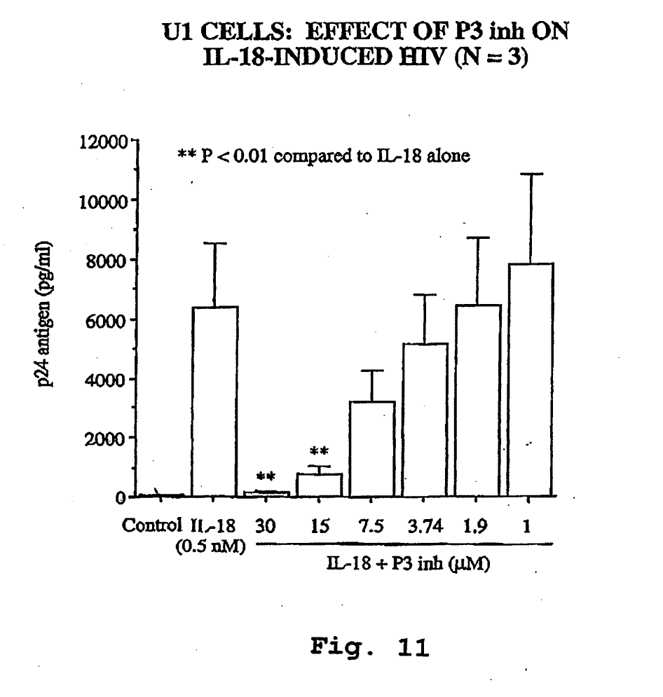

A series of drugs that may mimic AAT activity are tested for anti-HIV activity. These man-made drugs are made according to methods described in WO 98/24806, which discloses substituted oxadiazole, thiadiazole and triazole as serine protease inhibitors. In addition, U.S. Pat. No. 5,874,585 discloses substituted heterocyclic compounds useful as inhibitors of serine proteases; U.S. Pat. No. 5,869,455 discloses N-substituted derivatives; U.S. Pat. No. 5,861,380 discloses protease inhibitors-keto and di-keto containing ring systems; U.S. Pat. No. 5,807,829 discloses serine protease inhibitor-tripeptoid analogues; U.S. Pat. No. 5,801,148 discloses serine protease inhibitors-proline analogues; U.S. Pat. No. 5,618,792 discloses substituted heterocyclic compounds useful as inhibitors of serine proteases. Surprisingly, several of these drugs demonstrate anti-HIV activity at micromolar ranges. As a representative example shown in FIG. 11, a synthetic molecule (protease 3 inhibitor or P3 inh) mimicking AAT displays significant anti-HIV effect in the same experimental condition as in Example 1. As used hereinafter P3 inh is also designated as CE-2072 or (Benzyloxycarbonyl)-L-valyl-N-[1-(2-(3-methylbenzyl)-1,3,4-oxadiazolyncarbonyl)-2-(S)-methylpropyl]-L-prol-inamide. Methods of preparing P3 inh and derivatives thereof are disclosed in detail in U.S. Pat. No. 5,807,829 and incorporated by way of reference. CE 2072 along with AAT is tested in an assay that demonstrates the effect of these substances on NF-.kappa.B expression, which is induced by IL-18. Lane 4 in FIG. 13 shows band that corresponds to IL-18-induced NF-.kappa.B which is much larger than NF-.kappa.B in controls (lane 1) not stimulated by IL-18. In the presence of either AAT (lane 7) or AAT-mimicking synthetic molecule (lane 10) the NF-.kappa.B expression is reduced, indicating that these substances down-regulate NF-.kappa.B expression. This is a totally unexpected observation as these serine protease inhibitors are not known to interfere with NF-.kappa.B expression.

### Example 7

### Antiviral Activity of Man-Made Small Molecules

Without limiting to AAT and peptide derivatives of AAT, the compounds like oxadiazole, thiadiazole and triazole peptoids are preferred as they also show an equivalent antiviral activity in a mouse model as described in above Example 3. Anti-HIV effective doses are in a range from about 1.mu.g/kg to approximately 100 mg/kg. Specific examples of such oxadiazole, thiadiazole and triazole peptoids are molecules such as Benzyloxycarbonyl-L-valyl-N-[1-(2-(3-methylbenzyl)-1,3,4-oxadiazolyl]c-arbonyl)-2-(S)-methylpropyl]-L-prolinamide; Benzyloxycarbonyl-L-valyl-N-[1-(2-(5-(methyl)-1,3,4-oxadiazoly]carbony)-2-(S)-methylpropyl]-L-prolinamid-e; Benzyloxycarbonyl)-L-valyl-N-[1-(2-(5-(3-trifluoromethylbenzyl)-1,3,4-o-xadiazolyl]carbonyl)-2-(S)-methylpropyl]-L-prolinamide Benzyloxycarbonyl)-L-valyl-N-[1-(2-(5-(4-Dimethylaminobenzyl)-1,3,4-oxadi-azolyl]carbonyl)-2-(S)-methylpropyl]-prolinamide; Benzyloxycarbonyl)-L-valyl-N-[1-(2-(5-(1-napthylenyl)-1,3,4-oxadiazolyl]c-arbonyl)-2-(S)-methylpropyl]-prolinamide; (Benzyloxycarbonyl)-L-valyl-[1-(3-(5-(3,4-methylenedioxybenzyl)-1,2,4-oxadiazolyl]carbonyl)-2-(S)-methyl-propyl)-L-prolinamide; Benzyloxycarbonyl)-L-valyl-N-[1-(3-(5-(3,5-dimethyl-benzyl)-1,2,4-oxadiazolyl]carbonyl)-2-(S)-methylpropyl]-L-prolinamide; (Benzyloxycarbonyl)-L-valyl-N-[1-(3-(5-(3,5-dimethoxybenzyl)-1,2,4-oxadia-zolyl]carbonyl)-2-(S)-methylpropyl]-L-prolinamide; (Benzyloxycarbonyl)-L-v-alyl-N-[1-(3-(5-(3,5-ditrifluoromethylbenzyl)-1,2,4-oxadiazolyl]carbonyl)-2-(S)-methylpropyl]prolinamide; (Benzyloxycarbonyl)-L-valyl-N-[1-(3-(5-(3-methylbenzyl)-1,2,4-oxadiazolyl]carbonyl)-2-(S)-methylpropyl]-L-prolinamid-e; (Benzyloxycarbonyl)-L-valyl-N-[1-(3-(5-(biphenylmethine)-1,2,4-oxadiazo-lyl]carbonyl)-2-(S)-methylpropyl-L-prolinamide; (Benzyloxycarbonyl)-L-valy-1-N-[1-(3-(5-(4-phenylbenzyl)-1,2,4-oxadiazolyl]carbonyl)-2-(S)-methylprop-yl]-L-prolinamide; (Benzyloxycarbonyl)-L-valyl-N-[1-(3-(5-(3-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenoxybenzyl)-1,2,4-oxadiazoly-l]carbonyl)-2-(S)-methylpropyl]-L-prolinamide; (Benzyloxycarbonyl)-L-valyl-N-[1-(3-(5-(cyclohexylmethylene)-1,2,4-oxadiazolyl]carbonyl)-2-(S)-methyl-propyl]-L-prolinamide; (Benzyloxycarbonyl)-L-valyl-N-[1-(3-(5-(3-trifluoro-methyldimethylmethylene)-1,2,4-oxadiazolyl]carbonyl)-2-(S)-methylpropyl]-L-prolinamide; (Benzyloxycarbonyl)-L-valyl-N-[1-(3-(5-(1-napthylmethylene)-1,2,4-oxadiazolyl]carbonyl)-2-(S)-methylpropyl]-L-prolinamide; (Benzyloxycarbonyl)-L-valyl-N-[1-(3-(5-(3-pyridylmethyl)-1,2,4-oxadiazoly-l]carbonyl)-2-(S)-methylpropyl]-L-prolinamide; (Benzyloxycarbonyl)-L-valyl-N-[1-(3-(5-(3,5-diphenylbenzyl)-1,2,4-oxadiazolyl]carbonyl)-2-(S)-methylp-ropyl]-L-prolinamide; (Benzyloxycarbonyl)-L-valyl-N-[1-(3-(5-(4-dimethylam-inobenzyl)-1,2,4-oxadiazolyl]carbonyl)-2-(S)-methylpropyl]-prolinamide; 2-(5-[(Benzyloxycarbonyl)amino]-6-oxo-2-(4-fluorophenyl)-1,6-dihydro-1-py-rimidinyl]-N-[1-(3-(5-(3-trifluoromethylbenzyl)-1,2,4-oxadiazolyl]carbonyl)-(S)-2-methylpropyl]acetamide; 2-(5-Amino-6-oxo-2-(4-fluorophenyl)-1,6-di-hydro-1-pyrimidinyl]-N-[1-(3-(5-(3-trifluoromethylbenzyl)-1,2,4-oxadiazoly-1]carbonyl)-2-(S)-methylpropyl]acetamide; 2-(5-[(Benzyloxycarbonyl)amino]-6-oxo-2-(4-fluorophenyl)-1,6-dihydro-1-pyrimidinyl]-N-[1-(2-(5-(3-methylbe-nzyl)-1,3,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(2-(5-(3-methylbenzyl)-1,3,4-oxadiazolyl]carbonyl)-2-methylpropyl]acetamide; (Pyrrole-2-carbonyl)-N-(benzyl)glycyl-N-[1-(2-(5-(3-methylbenzyl)-1,3,4-o-xadiazolyl]carbonyl)-2-(S)-methylpropyl]amide; (Pyrrole-2-carbonyl)-N-(ben-zyl)glycyl-N-[1-(3-(5-(3-trifluoromethylbenzyl)]-1,2,4-oxadiazolyl)-(S)-me-thylpropyl]amide; (2S,5S)-5-Amino-1,2,4,5,6,7-hexahydroazepino-[3,2,1]-ind-ole-4-one-carbonyl-N-[1-(2-(5-(3-methylbenzyl)-1,3,4-oxadiazolyl]carbonyl)-(R,S)-2-methylpropyl]amide; BTD-[1-(2-(5-(3-methylbenzyl)-1,3,4-oxadiazol-yl]carbonyl)-2-(S)-methylpropyl]amide; (R,S)-3-Amino-2-oxo-5-phenyl-1,4,-b-enzodiazepine-N-[1-(2-(5-(3-methylbenzyl)-1,3,4-oxadiazolyl]carbonyl)-2-(S—)-methylpropyl]acetamide; (Benzyloxycarbonyl)-L-valyl-2-L-(2,3-dihydro-1H-indole)-N-[1-(2-(5-(3-methylbenzyl)-1,3,4-oxadiazolyl]carbonyl)-2-(S)-meth-ylpropyl]amide; (Benzyloxycarbonyl)-L-valyl-2-L-(2,3-dihydro-1H-indole)-N-[1-(3-(5-(3-trifluoromethylbenzyl)-1,2,4-oxadiazolyl]carbonyl)-2-(S)-methy-lpropyl]amide; Acetyl-2-L-(2,3-dihydro-1H-indole)-N-[1-(2-(5-(3-methylbenz-yl)-1,3,4-oxadiazolyl]carbonyl)-2-(S)-methylpropyl]amide; 3-(S)-(Benzyloxycarbonyl)amino)-.epsilon.-lactam-N-[1-(2-(5-(3-methylbenz-yl)-1,3,4-oxadiazolyl]carbonyl)-2-(S)-methylpropyl]acetamide; 3-(S)-(Amino)-.epsilon.-lactam-N-[1-(2-(5-(3-methylbenzyl)-1,3,4-oxadiazo-lyl]carbonyl)-2-(S)-methylpropyl]acetamide trifluoroacetic acid salt; 3-(S)-[(4-morpholinocarbonyl-butanoyl)amino]-.epsilon.-lactam-N-[1-(2-(5-(3-methylbenzyl)-1,3,4-oxadiazolyl]carbonyl)-2-(R,S)-methylpropyl]acetamid-e; 6-[4-Fluorophenyl]-.epsilon.-lactam-N-[1-(2-(5-(3-methylbenzyl)-1,3,4-o-xadiazolyl]carbonyl)-2-(S)-methylpropyl]acetamide; 2-(2-(R,S)-Phenyl-4-oxo-thiazolidin-3-yl]-N-[1-(2-(5-(3-methylbenzyl)-1,3,4-oxadiazolyl]carbonyl)-2-(S)-methylpropyl]acetamide; (2-(R,S)-phenyl-4-oxothiazolidin-3-yl]-N-[1-(2-(5-(3-methylbenzyl)-1,3,4-oxadiazolyl]hydroxymethyl)-2-(S)-methylpropyl-]acetamide; 2-(2-(R,S)-Benzyl-4-oxothiazolidin-3-yl-N-[1-(2-(5-(3-methylb-enzyl)-1,3,4-oxadiazolyl]carbonyl)-2-(S)-methylpropyl]acetamide; 2-(2-(R,S)-Benzyl-4-oxothiazolidin-3-yloxide]-N-[1-(3-(5-(3-trifluorometh-ylbenzyl)-1,2,4-oxadiazolyl]carbonyl)-2-(R,S-methylpropyl]acetamide; (1-Benzoyl-3,8-quinazolinedione)-N-[1-(2-(5-(3-methylbenzyl)-1,3,4-oxadia-zolyl]carbonyl)-2-(S)-methylpropyl]acetamide; (1-Benzoyl-3,6-piperazinedio-ne)-N-[1-(2-(5-(3-methylbenzyl)-1,3,4-oxadiazolyncarbonyl)-2-(S)-methylpr-opyl]acetamide; (1-Phenyl-3,6-piperazinedione)-N-[1-(2-(5-(3-methylbenzyl)-1,3,4-oxadiazolyncarbonyl)-2-(S)-methylpropyl]acetamide; [(1-Phenyl-3,6-piperazinedione)-N-[1-(3-(5-(3-trifluoromethylbenzyl)-1,2,-4-oxadiazolyl]carbonyl)]-2-(S)-methylpropyl]acetamide; 3-[(Benzyloxycarbonyl)amino]-quinolin-2-one-N-[1-(2-(5-(3-methybenzyl)-1,-3,4-oxadiazolyl]carbonyl)-2-(S)-methylpropyl]acetamide; 3-[(Benzyloxycarbonyl)amino]-7-piperidinyl-quinolin-2-one-N-[1-(2-(5-(3-m-ethybenzyl)-1,3,4-oxadiazolyl]carbonyl)-2-(S)-methylpropyl]acetamide; 3-(Carbomethoxy-quinolin-2-one-N-[1-(2-(5-(3-methybenzyl)-1,3,4-oxadiazol-yl]carbonyl)-2-(S)-methylpropyl]acetamide; 3-(Amino-quinolin-2-one)-N-[1-(2-(5-(3-methylbenzyl)-1,3,4-oxadiazolyl]carbonyl)-2-(S)-methylpropyl]aceta-mide; 3-[(4-Morpholino)aceto]amino-quinolin-2-one-N-[1-(2-(5-(3-methylbenz-yl)-1,3,4-oxadiazolyl]carbonyl)-2-(S)-methylpropyl]acetamide; 3,4-Dihydro-quinolin-2-one-N-[1-(2-(5-(3-methylbenzyl)-1,3,4-oxadiazolyn-carbonyl)-2-(S)-methylpropyl]acetamide; 1-Acetyl-3-(4-fluorobenzylidene)pi-perazine-2,5-dione-N-[1-(2-(5-(3-methylbenzyl)-1,3,4-oxadiazolyl]carbonyl)-2-(S)-methylpropyl]acetamide; 1-Acetyl-3-(4-dimethylaminobenzylidene)pipe-razine-2,5-dione-N-[1-(2-(5-(3-methylbenzyl)-1,3,4-oxadiazolyl]carbonyl)-2-(S)-methylpropyl]acetamide; 1-Acetyl-3-(4-carbomethoxybenzylidene)piperaz-ine-2,5-dione-N-[1-(2-(5-(3-methylbenzyl)-1,3,4-oxadiazolyncarbonyl)-2-(S)-methylpropyl]acetamide; 1-Acetyl-3-[(4-pyridyl)methylene]piperazine-2,5-dione-N [1-(2-(5-(3-methylbenzyl)-1,3,4-oxadiazolyl]carbonyl)-2-(S)-methyl-propyl]acetamide; 4-[1-Benzyl-3-(R)-benzyl-piperazine-2,5,-dione]-N-[1-(2-[5-(3-methylbenzyl)-1,3,4-oxadiazolyl]carbonyl)-2-(S)-methylpropyl]acetami-de; 4-[1-Benzyl-3-(S)-benzyl piperazine-2,5,-dione]-N-[1-(2-(5-(3-methylbe-nzyl)-1,3,4-Oxadiazolyl]carbonyl)-2-(S)-methylpropyl]acetamide; 4-[1-Benzyl-3(R)-benzylpiperazine-2,5,-dionei-N-[1-(3-(5-(3-trifluorometh-ylbenzyl)-1,2,4-oxadiazolyl]carbonyl)-2-(S)-methylpropyl]acetamide; 4-[1-Benzyl-3-(S)-benzylpiperazine-2,5,-dione]-N-[1-(3-(5-(3-trifluoromet-hylbenzyl)-1,2,4-oxadiazolyl]carbonyl)-2-(S)-methylpropyl]acetamide; 4-[1-Benzyl-3-(S)-benzyl piperazine-2,5,-dione]-N-[1-(3-(5-(2-dimethylami-noethyl)-1,2,4-oxadiazolyl]carbonyl)-2-(S)-methylpropyl]acetamide; 4-[1-Methyl-3-(R,S)-phenylpiperazine-2,5,-dione]-N [1-(3-(5-(3-trifluorom-ethylbenzyl)-1,2,4-oxadiazolyl]carbonyl)-2-(S)-methylpropyl]acetamide; 4-[[-Methyl-3-(R,S)-phenylpiperazine-2,5,-dione]-N-[1-(2-(5-(3-methylbenz-yl)-1,3,4-oxadiazolyl]carbonyl)-2-(S)-methylpropyl]acetamide; 4-[1-(4-Morpholinoethyl)-3-(R)-benzylpiperazine-2,5,-dione]-N-[1-(2-(5-(3-1-methylbenzyl)-1,3,4-oxadiazolyl]carbonyl)-2-(S)-methylpropyl]acetamide; 5-(R,S)-Phenyl-2,4-imidazolid inedione-N-[1-(2-(5-(3-methylbenzyl)-1,3,4-o-xadiazolyl]carbonyl)-2-(S)-methylpropyl]acetamide; 5-(R)-Benzyl-2,4-imidaz-olidinedione-N-[1-(2-(5-(3-methylbenzyl)-1,3,4-oxadiazolyncarbonyl)-2-(S)-methylpropyl]acetamide; 5-(S)-Benzyl-2,4-imidazolidinedione-N-[1-(2-(5-(3-1-methylbenzyl)-1,3,4-oxadiazolyl]carbonyl)-2-(S)-methylpropyl]acetamide; 5-(S)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 5-(R)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 1-Benzyl-4-(R)-benzyl-2,5-imidazolidinedione-N-[1-(2-(5-(3-methylbenzyl)-1,3,4-oxadiazolyl]carbonyl)-2-(S)-methylpropyl]acetamide; and 1-Benzyl-4-(R)-benzyl-2,5-imidazolidinedione-N-[1-(3-(5-(3-trifluoromethyl-(benzyl)-1,2,4-oxadiazolyncarbonyl)-2-(S)-methylpropyl]acetamide among others. Methods of making these molecules and derivatives thereof are well known in the art and can be found for example in U.S. Pat. Nos. 5,807,829; 5,891,852; 5,869,455; 5,861,380; and 5,801,148, which is incorporated herein by way of reference in its entirety.

Other small man-made molecules useful in this invention comprise phenylenedialkanoate esters, which are also effective in the mouse model. Specific examples of certain phenylenedialkanoate esters include but are not limited to: 2,2′-(1,4-phenylene)dibutyric acid; tert-butyl-3-chloro-pivaloate; dimethyl-2,2′-(1,4-phenylene)diisobutyrate-; 2,2′-(1,4-phenylene)diisobutyric acid; bis(sulfoxides); Obis(sulfones); and bis(4-(2′-carboxy-2′-methylpropylsulfonyl)phenyl)2,2′-(1,4-phenylene)-diisobutyrate among others. More specifically, U.S. Pat. No. 5,216,022 teaches other small molecules useful for the practice of this invention, including: Benzyloxycarbonyl-L-valyl-N-[1-(2-[5-(3-methylbenzyl)-1,3,4-ox-adiazolyl]carbonyl)-2-(S)-methylpropyl]-L-prolinamide (also known as CE-2072), Benzyloxycarbonyl-L-valyl-N-[1-(2-(3-methylbenzyl)-1,3,4-oxadia-zolyl]carbonyl)-2-(S)-methylpropyl]-L-prolinamide; Benzyloxycarbonyl-L-val-yl-N-[1-(2-(5-(methyl)-1,3,4-oxadiazoly]carbonyl)-2-(S)-methylpropyl]-L-pr-olinamide; Benzyloxycarbonyl)-L-valyl-N-[1-(2-(5-(3-trifluoromethylbenzyl)-1,3,4-oxadiazolyl]carbonyl)-2-(S)-methylpropyl]-L-prolinamide; (Benzyloxycarbonyl)-L-valyl-N-[1-(2-(5-(4-Dimethylaminobenzyl)-1,3,4-oxad-iazolyl]carbonyl)-2-(S)-methylpropyl]-L-prolinamide; Benzyloxycarbonyl)-L-valyl-N-[1-(2-(5-(1-napthylenyl)-1,3,4-oxadiazolyl]c-arbonyl)-2-(S)-methylpropyl]-L-prolinamide; (Benzyloxycarbonyl)-L-valyl-[1-(3-(5-(3,4-methylenedioxybenzyl)-1,2,4-oxadiazolyl]carbonyl)-2-(S)-methyl-propyl]-L-prolinamide; Benzyloxycarbonyl)-L-valyl-N-[1-(3-(5-(3,5-dimethyl-benzyl)-1,2,4-oxadiazolyl]carbonyl)-2-(S)-methylpropyl]-L-prolinamide; (Benzyloxycarbonyl)-L-valyl-N-[1-(3-(5-(3,5-dimethoxybenzyl)-1,2,4-oxadia-zolyncarbonyl)-2-(S)-methylpropyl]-L-prolinamide; (Benzyloxycarbonyl)-L-v-alyl-N-[1-(3-(5-(3,5-ditrifluoromethylbenzyl)-1,2,4-oxadiazolyl]carbonyl)-2-(S)-methylpropyl]-L-prolinamide; (Benzyloxycarbonyl)-L-valyl-N-[1-(3-(5-(3-methylbenzyl)-1,2,4-oxadiazolyncarbonyl)-2-(S)-methylpropyl]-L-prolina-mide; (Benzyloxycarbonyl)-L-valyl-N-[1-(3-(5-(biphenylmethine)-1,2,4-oxadi-azolyl]carbonyl)-2-(S)-methylpropyl]-L-prolinamide; (Benzyloxycarbonyl)-L-valyl-N-[4-(3-(5-(4-phenylbenzyl)-1,2,4-oxadiazolyl-]carbonyl)-2-(S)-methylpropyl]-L-prolinamide; (Benzyloxycarbonyl)-L-valyl-N-[1-(3-(5-(3-phenylbenzyl)-1,2,4-oxadiazolyl]carbonyl)-2-(S)-methylpropyl-]-L-prolinamide; (Benzyloxycarbonyl)-L-valyl-N-[1-(3-(5-(3-phenoxybenzyl)-1,2,4-oxadiazolyl]carbonyl)-2-(S)-methylpropyl]-L-prolinamide; (Benzyloxycarbonyl)-L-valyl-N-[1-(3-(5-(cyclohexylmethylene)-1,2,4-oxadia-zolyl]carbonyl)-2-(S)-methylpropyl]-L-prolinamide; (Benzyloxycarbonyl)-L-v-alyl-N-[1-(3-(5-(3-trifluoromethyldimethylmethylene)-1,2,4-oxadiazolyl]car-bonyl)-2-(S)-methylpropyl]-L-prolinamide; (Benzyloxycarbonyl)-L-valyl-N-[1-(3-(5-(1-napthylmethylene)-1,2,4-oxadiazolyl]carbonyl)-2-(S)-methylpropyl-]-L-prolinamide; (Benzyloxycarbonyl)-L-valyl-N-[1-(3-(5-(3-pyridylmethyl)-1,2,4-oxadiazolyl]carbonyl)-2-(S)-methylpropyl]-L-prolinamide; (Benzyloxycarbonyl)-L-valyl-N-[1-(3-(5-(3,5-diphenylbenzyl)-1,2,4-oxadiaz-olyl]carbonyl)-2-(S)-methylpropyl]-L-prolinamide; (Benzyloxycarbonyl)-L-va-lyl-N-[1-(3-(5-(4-dimethylaminobenzyl)-1,2,4-oxadiazolyl]carbonyl)-2-(S)-m-ethylpropyl]-L-prolinamide; 2-(5-[(Benzyloxycarbonyl)amino]-6-oxo-2-(4-flu-orophenyl)-1,6-dihydro-1-pyrimidinyl]-N-[1-(3-(5-(3-trifluoromethylbenzyl)-1,2,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(3-(5-(3-trifluoromethylbenzyl)-1,2,4-oxadiazolyl]carbonyl)-2-(S)-methylpropyl]-acetamide; 2-(5-[(Benzyloxycarbonyl)amino)-6-oxo-2-(4-fluorophenyl)-1,6-di-hydro-1-pyrimidinyl]-N-[1-(2-(5-(3-methylbenzyl)-1,3,4-oxadiazolyl]carbony-l)-(S)-2-methylpropyl]acetamide; 2-(5-Amino-6-oxo-2-(4-fluorophenyl)-1,6-d-ihydro-1-pyrimidinyl]-N-[1-(2-(5-(3-methylbenzyl)-1,3,4-oxadiazolyl]carbon-yl)-2-methylpropyl]acetamide; (Pyrrole-2-carbonyl)-N-(benzyl)glycyl-N-[1-(2-(5-(3-methylbenzyl)-1,3,4-oxadiazolyl]carbonyl)-2-(S)-methylpropyl]amide-; (Pyrrole-2-carbonyl)-N-(benzyl)glycyl-N-[1-(3-(5-(3-trifluoromethylbenzy-l)]-1,2,4-oxadiazolyl)-(S)-methylpropylamide; (2S,5S)-5-Amino-1,2,4,5,6,7-hexahydroazepino-[3,2,1]-indole-4-one-carbonyl-N-[1-(2-(5-(3-methylbenzyl)-1,3,4-oxadiazolyl]carbonyl)-(R,S)-2-methylpropyl]amide BTD-[1-(2-(5-(3-methylbenzyl)-1,3,4-oxadiazolyl]carbonyl)-2-(S)-methylpro-pyl]amide; (R,S)-3-Amino-2oxo-5-phenyl-1,4,-benzodiazepine-N-[1-(2-(5-(3-m-ethylbenzyl)-1,3,4-oxadiazolyl]carbonyl)-2-(S)-methylpropyl]acetamide; (Benzyloxycarbonyl)-L-valyl-2-L-(2,3-dihydro-1H-indole)-N-[1-(2-(5-(3-met-hylbenzyl)-1,3,4-oxadiazolyl]carbonyl)-2-(S)-methylpropyl]amide; (Benzyloxycarbonyl)-L-valyl-2-L-(2,3-dihydro-1H-indole)-N-[1-(3-(5-(3-tri-fluoromethylbenzyl)-1,2,4-oxadiazoly]carbonyl)-2-(S)-methylpropyl]amide; Acetyl-2-L(2,3-dihydro-1H-indole)-N-[1-(2-(5-(3-methylbenzyl)-1,3,4-oxadi-azolyl]carbonyl)-2-(S)-methylpropyl]amide; 3-(S)-(Benzyloxycarbonyl)amino)-.epsilon.-lactain-N-[1-(2-(5-(3-methylbenzyl)-1,3,4-oxadiazolyl]carbonyl)-2-(S)-methylpropyl]acetamide; 3-(S)-(Amino)-.epsilon.-lactam-N-[1-(2-(5-(3-methylbenzyl)-1,3,4-oxadiazolyl]carbonyl)-2-(S)-methylpropyl]acetamide trifluoroacetic acid salt; 3-(S)-[(4-morpholinocarbonyl-butanoyl)amino]-.-epsilon.-lactan-N-[1-(2-(5-(3-methylbenzyl)-1,3,4-oxadiazolyl]carbonyl)-2-(R,S)-methylpropyl]acetamide; 644-Fluorophenyl].epsilon.-lactam-N-[1-(2-(5-(3-methylbenzyl)-1,3,4-oxadiazolyl]carbonyl)-2-(S)-methylpropyl]acetami-de; 2-(2-(R,S)-Phenyl-4-oxothiazolidin-3-yl]-N-[1-(2-(5-(3-methylbenzyl)-1-3,4-oxadiazolyl]carbonyl)-2-(S)-methylpropyl]acetamide; 2-(2-(R,S)-phenyl-4-oxothiazolidin-3-yl]-N-[1-(2-(5-(3-methylbenzyl)-1,3,-4-oxadiazolyl]hydroxymethyl)-2-(S)-methylpropyl]acetamide; 2-(2-(R,S)-Benzyl-4-oxothiazolidin-3-yl]-N-[1-(2-(5-(3-methylbenzyl)-1,3,-4-oxadiazolyl]carbonyl)-2-(S)-methylpropyl]acetamide; 2-(2-(R,S)-Benzyl-4-oxothiazolidin-3-yl oxide]-N-[1-(3-(5-(3-trifluoromet-hylbenzyl)-1,2,4-oxadiazolyl]carbonyl)-2-(R,S-methylpropyl]acetamide; (1-Benzoyl-3,8-quinazolinedione)-N-[1-(2-(5-(3-methylbenzyl)-1,3,4-oxadia-zolyl]carbonyl)-2-(S)-methylpropyl]acetamide; (1-Benzoyl-3,6-piperazinedio-ne)-N-[1-(2-(5-(3-methylbenzyl)-1,3,4-oxadiazolyl]carbonyl)-2-(S)-methylpr-opyl]acetamide; (1-Phenyl-3,6-piperazinedione)-N-[1-(2-(5-(3-methylbenzyl)-1,3,4-oxadiazolyl]carbonyl)-2-(S)-methylpropyl]acetamide; [(1-Phenyl-3,6-piperazinedione)-N-[1-(3-(5-(3-trifluoromethylbenzyl)-1,2,-4-oxadiazolyl]carbonyl)]-2-(S)-methylpropyl]acetamide; 3-[(Benzyloxycarbonyl)amino]-quinolin-2-one-N-[1-(2-(5-(3-methylbenzyl)-1-3,4-oxadiazolyl]carbonyl)-2-(S)-methylpropyl]acetamide; 3-[(Benzyloxycarbonyl)amino]-7-piperidinyl-quinolin-2-one-N-[1-(2-(5-(3-m-ethylbenzyl)-1,3,4-oxadiazolyl]carbonyl)-2-(S)-methylpropyl]acetamide; 3-(Carbomethoxy-quinolin-2-one-N-[1-(2-(5-(3-methybenzyl)-1,3,4-oxadiazol-yl]carbonyl)-2-(S)-methylpropyl]acetamide; 3-(Amino-quinolin-2-one)-N-([1-(2-(5-(3-methylbenzyl)-1,3,4-oxadiazolyl]carbonyl)-2-(S)-methylpropyl]acet-amide; 3-[(4-Morpholino)aceto]amino-quinolin-2-one-N-[1-(2-(5-(3-methylben-zyl)-1,3,4-oxadiazolyl]carbonyl)-2-(S)-methylpropyl]acetamide; 3,4-Dihydro-quinolin-2-one-N-[1-(2-(5-(3-methylbenzyl)-1,3,4-oxadiazolyl]-carbonyl)-2-(S)-methylpropyl]acetamide; 1-Acetyl-3-(4-fluorobenzylidene)pi-perazine-2,5-dione-N-[1-(2-(5-(3-methylbenzyl)-1,3,4-oxadiazolyl]carbonyl)-2-(S)-methylpropyl]acetamide; 1-Acetyl-3-(4-dimethylaminobenzylidene)pipe-razine-2,5-dione-N-[1-(2-(5-(3-methylbenzyl)-1,3,4-oxadiazolyl]carbonyl)-2-(S)-methylpropyl]acetamide; 1-Acetyl-3-(4-carbomethoxybenzylidene)piperaz-ine-2,5-dione-N-[1-(2-(5-(3-methylbenzyl)-1,3,4-oxadiazolyl]carbonyl)-2-(S)-methylpropyl]acetamide; 1-Acetyl-3-[(4-pyridyl)methylene]piperazine-2,5-dione-N-[1-(2-(5-(3-methylbenzyl)-1,3,4-oxadiazolyl]carbonyl)-2-(S)-methyl-propyl]acetamide; 4-[1-Benzyl-3-(R)-benzyl-piperazine-2,5-dione]-N-[1-(2-[5-(3-methylbenzyl)-1,3,4-oxadiazolyl]carbonyl)-2-(S)-methylpropyl]acetami-de; 4-[1-Benzyl-3-(S)-benzyl piperazine-2,5,-dione]-N-[1-(2-(5-(3-methylbe-nzyl)-1,3,4-oxadiazolyl]carbonyl)-2-(S)-methylpropyl]acetamide; 4-[1-Benzyl-3(R)-benzylpiperazine-2,5,-dione]-N-[1-(3-(5-(3-trifluorometh-ylbenzyl)-1,2,4-oxadiazolyl]carbonyl)-2-(S)-methylpropyl]acetamide; 4-[1-Benzyl-3-(S)-benzylpiperazine-2,5,-dione]-N-[1-(3-(5-(3-trifluoromet-hylbenzyl)-1,2,4-oxadiazolyl]carbonyl)-2-(S)-methylpropyl]acetamide; 4-[1-Benzyl-3-(S)-benzyl piperazine-2,5,-dione]-N-[1-(3-(5-(2-dimethylami-noethyl)-1,2,4-oxadiazolyl]carbonyl)-2-(S)-methylpropyl]acetamide; 4-[1-Methyl-3-(R,S)-phenylpiperazine-2,5,-dione]-N-[1-(3-(5-(3-trifluorom-ethylbenzyl)-1,2,4-oxadiazolyl]carbonyl)-2-(S)-methylpropyl]acetamide; 4-[[-Methyl-3-(R,S)-phenylpiperazine-2,5,-dione]-N-[1-(2-(5-(3-methylbenz-yl)-1,3,4-oxadiazolyl]carbonyl)-2-(S)-methylpropyl]acetamide; 4-[1-(4-Morpholinoethyl)-3-(R)-benzylpiperazine-2,5,-dione]-N41-(2-(5-(3-methylbenzyl)-1,3,4-oxadiazolyncarbonyl)-2-(S)-methylpropyl]acetamide; 5-(R,S)-Phenyl-2,4-imidazolidinedione-N-[1-(2-(5-(3-methylbenzyl)-1,3,4-o-xadiazolyl]carbonyl)-2-(S)-methylpropyl]acetamide; 5-(R)-Benzyl-2,4-imidaz-olidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 5-(R)-Benzyl-2,4-imidazolidinedione-N-[1-(3-(5-(3-trifluoromethylbenzyl)-1,2,4-oxadiazolyncarbonyl)-2-(S)-methylpropyl]acetamide; 1-Benzyl-4-(R)-benzyl-2,5-imidazolidinedione-N-[1-(2-(5-(3-methylbenzyl)-1,3,4-oxadiazolyl]carbonyl)-2-(S)-methylpropyl]acetamide; and 1-Benzyl-4-(R)-benzyl-2,5-imidazolidinedione-N-[1-(3-(5-(3-trifluoromethyl-lbenzyl)-1,2,4-oxadiazolyl]carbonyl)-2-(S)-methylpropyl]acetamide among others. Methods of making these molecules and derivatives thereof are well known in the art and can be found in aforementioned U.S. Pat. No. 5,216,022, which is incorporated herein by way of reference in its entirety.

Likewise, U.S. Pat. No. 5,869,455 discloses N-substituted derivatives; U.S. Pat. No. 5,861,380 protease inhibitors-keto and di-keto containing ring systems; U.S. Pat. No. 5,807,829 serine protease inhibitor-tripeptoid analogues; U.S. Pat. No. 5,801,148 serine protease inhibitors-proline analogues; U.S. Pat. No. 5,618,792 substituted heterocyclic compounds useful as inhibitors of serine proteases. These patents and PCT publications and others as listed infra are enclosed herein by reference. Other equally advantageous molecules, which may be used instead of .alpha.sub.1-antitrypsin or in combination with .alpha.sub.1-antitrypsin are contemplated such as in WO 98/20034 disclosing serine protease inhibitors from fleas. Without limiting to this single reference one skilled in the art can easily and without undue experimentation adopt compounds such as in WO98/23565 which discloses aminoguanidine and alkoxyguanidine compounds useful for inhibiting serine proteases; WO98/50342 discloses bis-aminomethylcarbonyl compounds useful for treating cysteine and serine protease disorders; WO98/50420 cyclic and other amino acid derivatives useful for thrombin-related diseases; WO 97/21690 D-amino acid containing derivatives; WO 97/10231 ketomethylene group-containing inhibitors of serine and cysteine proteases; WO 97/03679 phosphorous containing inhibitors of serine and cysteine proteases; WO 98/21186 benzothiazo and related heterocyclic inhibitors of serine proteases; WO 98/22619 discloses a combination of inhibitors binding to P site of serine proteases with chelating site of divalent cations; WO 98/22098 a composition which inhibits conversion of pro-enzyme CPP32 subfamily including caspase 3 (CPP32/Yama/Apopain); WO 97/48706 pyrrolo-pyrazine-diones; WO 97/33996 human placental bikunin (recombinant) as serine protease inhibitor; WO 98/46597 complex amino acid containing molecule for treating viral infections and conditions disclosed hereinabove.

Other compounds having serine protease inhibitory activity are equally suitable and effective including but not limited to tetrazole derivatives as disclosed in WO 97/24339; guanidinobenzoic acid derivatives as disclosed in WO 97/37969 and in a number of U.S. Pat. Nos. 4,283,418; 4,843,094; 4,310,533; 4,283,418; 4,224,342; 4,021,472; 5,376,655; 5,247,084; and 5,077,428; phenylsulfonylamide derivatives represented by general formula in WO 97/45402; novel sulfide, sulfoxide and sulfone derivatives represented by general formula in WO 97/49679; novel amidino derivatives represented by general formula in WO 99/41231; other amidinophenol derivatives as disclosed in U.S. Pat. Nos. 5,432,178; 5,622,984; 5,614,555; 5,514,713; 5,110,602; 5,004,612; and 4,889,723 among many others.

In summary, the Examples recited hereinabove show that compounds exhibiting AAT activity such as AAT, peptides derived analogous or homologous to C-terminal end of AAT, and man-made synthetic molecules mimicking AAT action, display herpes virus-suppressive effects in vitro and in vivo.

### Example 7

Synergy of AAT and AAT-Related Molecules with Anti-HIV Drugs

AAT and AAT-related molecules displaying AAT activity are tested for possible utility as a combination therapy with established anti-HIV drugs. Among these compositions are nucleoside reverse transcriptase (RT) inhibitors such as Retrovir (AZT/zidovudine; Glaxo Wellcome); Epivir (3TC, lamivudine; Glaxo Wellcome); Videx (ddl/didanosine; Bristol-Myers Squibb); Hivid (ddC/zalcitabine; Hoffmann-La Roche); Zerit (d4T/stavudine; Bristol-Myers Squibb); Ziagen (abacavir, 1592U89; Glaxo Wellcome); Hydrea (Hydroxyurea/HO; Bristol-Myers Squibb) and non-nucleoside reverse transcriptase inhibitors (NNRTIS) such as Viramune (nevirapine; Roxane Laboratories); Rescriptor (delavirdine; Pharmacia & Upjohn); Sustiva (efavirenz, DMP-266; DuPont Merck); Preveon (adefovir dipivoxil, bis-POM PMEA; Gilead). Also tested are aspartyl protease inhibitors (PI's) including Fortovase (saquinavir; Hoffmann-La Roche); Norvir (ritonavir; Abbott Laboratories); Crixivan (indinavir; Merck & Company); Viracept (nelfinavir; Agouron Pharmaceuticals); and Angenerase (amprenavir/141 W94; Glaxo Wellcome). The presence of the compositions of the present invention enhances the antiviral effect of above-listed drugs.

In summary, the studies presented supra demonstrate HIV-1-suppressive activity of AAT and related compounds with AAT activity in all three in vitro models; U1 cells, PBMC, and MAGI cells. To anyone skilled in the art it is obvious that these models closely relate to the in vivo situation. This is further supported by the commercial and clinical success of existing, publicly available anti-HIV drugs (listed in Example 6) which were all initially tested in similar in vitro models. The results from such models are highly and invariably predictable of the success or failure in clinical setting. Experiments conducted in U1 cells establish the blockade of HIV-1 production in a chronic infection model. This inhibitory effect is observed for all stimuli tested, including inflammatory cytokines (IL-18, IL-6, TNF) LPS and hyperosmolarity. The inhibitory effect is potent, with a range of inhibition of 73-100%. Since AAT is not known to have intracellular antiprotease activity (size of AAT molecule is too large to cross the plasma membrane), these results suggest the existence of an extracellular protease(s) required for virion production. Although pro-inflammatory cytokines and LPS are not known to physically interact with AAT, we excluded this mechanism of AAT inhibition by hyperosmolarity-induced HIV-1. Hyperosmolarity established by adding NaCl to U1 cell cultures increased p24 antigen production. As shown in FIG. 10, 60 mM NaCl added to culture resulted in a 26-fold increase in p24 concentration compared to control. This increase is completely inhibited in the presence of 5 mg/ml AAT.

Results obtained in HIV-1-infected PBMC demonstrate several characteristics of AAT inhibition. Experiments are performed in PBMC from three donors infected in the absence or presence of AAT during infection. The presence of AAT during infection did not affect p24 antigen production following removal of AAT and 4 days of culture in medium alone. Therefore, any effects of AAT at the time of infection are reversible. However, AAT effects during the infection period are established by the enhancement of AAT effect when added to PBMC following infection and cultured for 4 days. Enhancement of 4 day AAT effect is manifested by a larger maximal suppression and by suppression at lower AAT concentrations. Maximal p24 reductions in PBMC exposed to AAT for 4 days are 46% and 71% for cells infected in the absence or presence of AAT, respectively. For cells infected in the absence of AAT, a significant suppressive effect is observed for post-infection AAT added at 5 and 4 mg/ml, and for cells infected in the presence of AAT significant effect is obtained at 5, 4, 3, and 2 mg/ml. Considered together, these data indicate a reversible enhancing effect of AAT when present at the time of PBMC infection.

Experiments performed in MAGI-CCR-5 cells (FIGS. 3 and 4) indicate inhibitory effects of AAT and related compounds on early infection-associated events. The observed dose-dependent effect is maximal at 5 mg/ml AAT, where 76% inhibition is observed compared to control (HIV-1 added in the absence of AAT). Therefore, AAT inhibits HIV-1 events prior to integration into the host-cell genome (cell-surface receptor binding, internalization, integration, uncoating, reverse transcription, translation and protein processing and tat activation).

Also, AAT, peptides derived analogous or homologous to C-terminal end of AAT, and representative man-made synthetic molecules mimicking AAT action, display HIV-1-suppressive effects operative during both early (PBMC and MAGI-CCR-5 cell results) and late (U1 cell results) events associated with HIV-1 infection. Unexpectedly, the synergy appears to exist between known AIDS drugs belonging to RT and PI classes and compositions of this invention, which belong to unrelated class of inhibitors, i.e., serpins.

Throughout this application various publications and patents are referenced. The disclosures of these publications and patents in their entireties are hereby incorporated by reference into this application in order to more fully describe the state of the art to which this invention pertains.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

### Methods Continued

Blood Draw: In certain exemplary methods, first blood was drawn into syringes containing heparin (10 20 U/mL, or use commercial heparinized sterile tubes) and second, cells were separated. In one particular example, 1.0 mL blood provides 1×10⁶ PBMC and about 2.5×10⁶ PBMC per tube were used for these experimental examples.

Cell Separation can include for example:

a) 20 mL sterile saline is added to 50 ml polypropylene tubes.
b) Put 10 mL whole blood into each 50 mL polypropylene tube.
c) Underlay each tube with 10 mL ficoll hypaque using a pipette or a spinal needle, proceed at a rate of about 1 minute per underlay.
d) Centrifuge the tubes at 1,250 rpm (=400 g)×40 minutes at room temperature.
e) Harvest PBMC layers from 2 tubes using a 10 ml pipette and place into a fresh 50 ml polypropylene tube.
f) Fill tubes to 50 mL with saline.
g) Centrifuge tubes at 1,000 rpm×10 minutes at room temperature.
h) Decant supernatant.
i) Resuspend cells in 10 mL saline and combine all tubes into as few tubes as possible.
j) Fill tube(s) to 50 mL with saline.
k) Centrifuge tube(s) at 1,000 rpm×10 minutes at room temperature.
l) Decant supernatant.
m) Resuspend the cells with a pipette in EXACTLY 10 mL of saline.
n) Count cells in a hemacytometer (total #).
o) Add an additional 40 mL of saline to the tube(s); each now contains 50 mL liquid.
p) Centrifuge the tubes at 1,000 rpm×10 minutes at room temperature.
q) Decant supernatant.
r) Resuspend cells at 1×106/mL in sterile R3 tissue culture medium (RPMI 1640 medium with 20% [vol/vol] heat-inactivated fetal bovine serum, 5% [vol/vol] Interleukin (IL)-2 and penicillin 100 units/ml+streptomycin 100 μg/ml) supplemented 3.3 μg/ml PHA.

Third, cells were induced into blast phase by culture by incubation for 2 days (37° C., 5% CO₂) in sterile tissue culture flasks.

Fourth, PBMC were then infected with HIV: After the 2 days of blasting/incubation, the cells were counted and the number of PBMC was determined for infecting with HIV. A cell suspension was aliquoted into a polypropylene tube, then centrifuged into a pellet. Then, the tubes are inverted right away, preserving the cell pellet: approximately 300 μl of liquid remains with the cell pellet. The virus of choice was added. For the X4/T tropic A018A strain, the PBMC was infected with 200 TCID50 per 1 million PBMC. For the R5/M tropic virus strain, 300 TCID50 per 1 million PBMC was used for infection. After adding the virus, the virus was resuspended vigorously with a pipetter and vortex as well. Then the cells were incubated in the 50 ml polypropylene tube (loose cap) for 3 hrs in an incubator. c) After 3 hrs of incubation, the infected PBMC were washed with RPMI or with PBS (resuspend with a vacuum pipetter), then centrifuge. No significant amount of virus remains after this step. d) The infected PBMC was resuspended at 2×106 per ml in non-blasting R3 medium=R3 medium as above but without PHA. (=RPMI+10% FCS+5% IL 2).

Fifth, the cell suspension was aliquoted into 24-well polystyrene plates at a final concentration of 1×10⁶ per ml. Sixth, a time zero sample was created by taking a 250 μl aliquot of cell suspension at 2×10⁶ cells per ml and add this into a 1.5 ml Eppendorf tube. Add to this 250 μl of medium and 50 μl of (10% vol/vol) Triton X 100. The sample is froze immediately at −70° C. and assay later for p24 antigen as the time 0 specimen. Seventh, 250 μl of cell suspension was added to each well with an additional 250 μl of R3 medium alone (Spontaneous, or AAT=0), or R3 that contains AAT (either Aralast® or Zemaira®) at twice the final desired concentrations. The final volume of each culture is 500 μl. Eighth, the tissue culture plates were incubated with cell cultures in an incubator (37° C., 5% CO₂), for 4 days, then add 50 μl of 10% (vol/vol) Triton X 100 to make a final Triton X 100 concentration of 1% vol/vol. Finally HIV p24 antigen was quantified using an ELISA assay.

As demonstrated in exemplary FIG. 1, Aralast substantially induced HIV inhibition at all concentrations tested (compared to AAT=0 cultures), with nearly 100% suppression observed using Aralast at 3.0 8.0 mg/ml, and about 50% HIV suppression using Aralast at 1.0 mg/ml. In contrast, Zemaira AAT demonstrated minimal HIV suppression at 7.0 mg/ml, and near complete suppression was obtained at 15.0 mg/ml. In this exemplary method, there was a large difference in dose response demonstrating that Aralast is more potent than Zemaira as an inhibitor of HIV infection in primary PBMC. Since Aralast and Zemaira are quantified by biological activity (1.0 mg Aralast=1.0 mg Zemaira=1.0 mg of serine protease inhibitor activity), this experiment indicates that the ability of AAT to suppress HIV is independent of serine protease inhibition. If the serine protease inhibitor function of AAT accounts for the HIV suppression, Aralast and Zemaira would inhibit HIV production equivalently.

### Procedures for Heat Inactivation (HI) of AAT:

In another exemplary method, a predetermined volume (e.g. 2 mls) of a stock solution such as 20 mg/ml of AAT (e.g. Aralast) was placed in a test tube. The stock sample was heat treated in boiling water (95° C.) for 30 min. The solution was allowed to cool. Then the heated solution was transferred back to eppendorf tube(s). If any volume has boiled off (usually about 10%), the volume is replaced with a solution to near original volume using for example, PBS. Then the solution is tested for remaining serine protease activity using a serine protease inhibitor assay. It was demonstrated that no significant serine protease inhibitor activity could be detected for up to 3 days later (data not shown).

### Example 8

Elastase assay: In one example, an enzymatic assay of elastase biological activity based on Bieth et al (Bieth J, Spiess B, Wermuth CG, 1974, Biochemical Medicine, vol 11, pp 350-357) was used to compare AAT and heat-inactivated (HI) AAT.

Elastase-induced hydrolysis of the N-Succinyl-Ala-Ala-Ala-p-nitroanalide serine protease substrate (e.g., Sigma, St. Louis, Mo.) liberates p-nitroanaline, which can be measured at an absorbance of 410 nm. Elastase (e.g., Sigma) is diluted to 20 μg/ml in 100 mM tris-HCl, pH 8.0. Ten microliters AAT (at 20 mg/ml) or PBS (Control without AAT, set at 100% elastase activity) is mixed with 50 μl of diluted elastase and incubated for 20 mins at 25° C. Ten microliters of the alpha-1-antitrypsin/elastase or PBS/elastase solutions are added to 180 μl of substrate (alpha-1-antitrypsin, which was diluted to 135 μg/ml with 100 nM Tris HCl. pH 8.0) and transferred into wells of a 96 well flat bottom plate. An increase in absorbance (A) 410 nm (which indicated elastase-induced generation of p-nitroanaline) was measured serially over a 5 minute time period. Elastase alone was used as a Control (set at 100% elastase activity). The presence of a serine protease inhibitor (e.g., AAT) blocks elastase activity and suppresses liberation of p-nitroanaline (quantified as A410).

Elastase alone (no AAT) data not shown processed the N-Succinyl-Ala-Ala-Ala-p-nitroanalide substrate, which generated a step increase in absorbance (A410, curve labeled Elastase). Combining native (NOT heat-inactivated) AAT ablated elastase processing of the N-Succinyl-Ala-Ala-Ala-p-nitroanalide substrate and blocked the increase in A410 nm (curve labeled AAT+Elastase). In marked contrast, combining HIAAT with elastase produced a curve similar to that of elastase alone. This demonstrated that HIAAT possessed no detectable elastase neutralizing activity, since the elastase-induce generation of p-nitroanaline due to processing of the substrate N-Succinyl-Ala-Ala-Ala-p-nitroanalide was unaffected (see curve labeled HIAAT+Elastase and compare to curve labeled Elastase).

### Example 9

### Heat-Inactivated AAT (ΔAAT) Retains Biological Activity in Human Primary Fibroblasts

In another exemplary method, human fetal foreskin fibroblasts were obtained. Fibroblasts were grown in culture medium (e.g. RPMI 1640 medium with 10% [vol/vol] heat inactivated fetal bovine serum) in 150 mL polystyrene tissue culture flasks (Falcon, Lincoln Park, N.J.) and incubated at 37° C. and 5% CO₂ until confluent. The cells were detached using trypsin and split into 24-well polystyrene cell culture plates. The cells were then allowed to grow to confluence in these plates for 3-5 days before the actual experiments were performed. Cells were incubated (37° C., 5% CO₂) in culture medium alone (Control), AAT alone, or with heat inactivated AAT (ΔAAT). After 24 hours of incubation (37° C., 5% CO₂) supernatants were removed and frozen (−70° C.) until assay for IL-6 (data not shown).

### Example 10

### Anti-HIV Effect of AAT

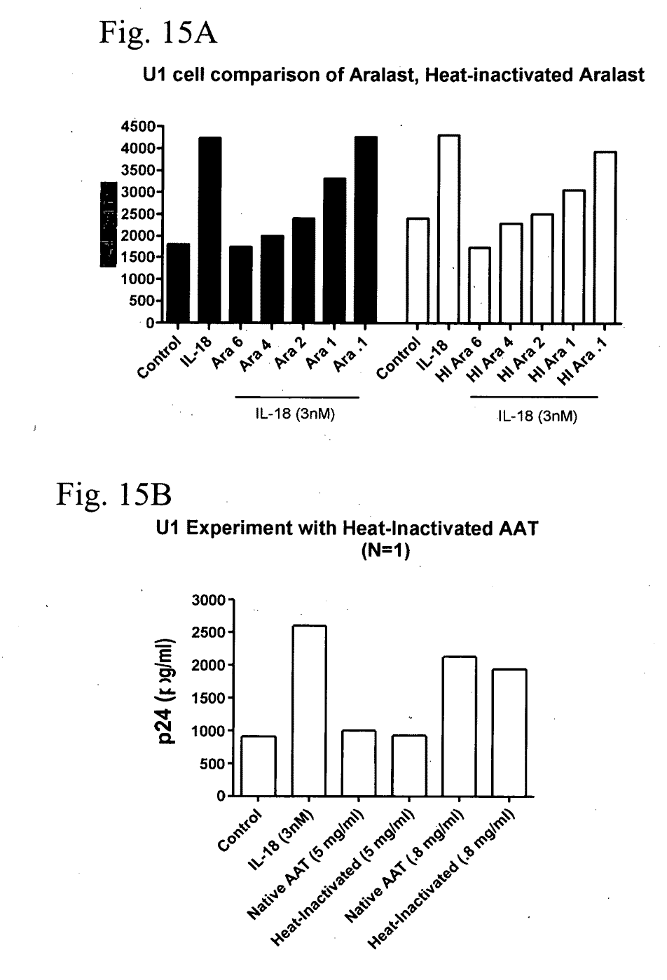

In one exemplary method it was demonstrated that AAT and HIAAT (ΔAAT) inhibit HIV production in chronically infected U1 cells. In these exemplary experiments, U1 cells were cultured at a density of 1×10⁶ cells per ml in 500 μl of medium consisting of RPMI 1640 medium with 10% [vol/vol] heat inactivated fetal calf serum, with penicillin 100 units/ml+streptomycin 100 μg/ml. Cells were cultured in wells of a polystyrene tissue culture plate with medium alone (control), with medium containing stimulus alone (3 nM IL 18), or with stimulus in the presence of AAT (FIG. 15A, left panel) or heat inactivated AAT (FIG. 15A, right panel). AAT was added to cultures 1.0 hr prior to the addition of IL-18 (interleukin 18) stimulus. Cultures were incubated for 24 hrs (37° C., 5% CO₂), and then lysed with 1% (vol/vol) triton X 100 and then the lysates were assayed for HIV p24 antigen using an ELISA. As shown in FIGS. 15A and 15B, IL-18 stimulated an increase in HIV production compared to medium alone (control) cultures. Stimulating U1 cell cultures with IL-18 in the presence of either unaltered (FIG. 15A, left panel) AAT or with heat inactivated AAT (FIG. 15A, right panel) resulted in dose dependent inhibition of stimulated HIV production. Comparing native with heat inactivated AAT showed very similar inhibition of p24 production. For both native and heat inactivated AAT, nearly complete HIV suppression induced by IL 18 was observed using AAT concentrations of 4 and 6 mg/ml. These results suggest very similar HIV suppression in this chronic infection model using native or heat inactivated AAT. Another experiment was performed using 0.8 or 5 mg/ml of AAT or HI AAT (FIG. 15B). For both native and heat inactivated AAT, nearly complete HIV suppression induced by IL 18 was observed using AAT of HI AAT concentrations of 5 mg/ml but not at 0.8 mg/ml. Since heat inactivation of AAT using our protocol ablates AAT serine protease inhibitory function (as documented in by an in vitro serine protease neutralization assay, data not shown), these results suggest that AAT suppression of HIV in these studies does not depend on the serine protease inhibitor function of AAT.

### Example 11

In another exemplary method, AAT (Native AAT) and HI AAT activity were analyzed for their effects on lethal toxin-induced cytotoxicity in RAW 264.7 cells (N=5). In this example, all cultures received a lethal toxin (100 ng/ml protective antigen+40 ng/ml lethal factor); p<0.001 compared to Control. This exemplary study was used to demonstrate HI AAT versus native AAT treatments on cells exposed to anthrax.

RAW 264.7 cells were cultured in medium (RPMI 1640 medium+10 heat-inactivated FBS with 100 units/ml penicillin and 100 μg/ml streptomycin) containing lethal toxin (LT) alone (control), or in medium containing LT and AAT. AAT was added 1 hr prior to addition of LT. Three hrs after addition of LT, cell culture supernatant was assayed for cytotoxicity using an LDH release assay (Promega, Madison, Wis.). Cells cultured in LT alone (Control, closed bar) demonstrated cytotoxicity that produced a mean of approximately 0.25 OD units (LDL OD units on the vertical axis represents increasing amounts of cytotoxicity. Five mg/ml native (not heat-inactivated) AAT significantly reduced the LT-induced cytotoxicity in the RAW 264.7 cells), whereas 3.0 mg/ml native AAT did not inhibit LT cytotoxicity. As shown in the same figure, HI AAT replicated the native AAT results almost identically, with 5.0 mg/ml HI AAT significantly reducing LT-induced cytotoxicity. In this Figure results from 5 separate experiments are shown (mean±SEM), and *** indicates p<0.001 compared to Control (no AAT, closed bar). These data show that HI AAT is equivalent to native AAT as an inhibitor of anthrax cytotoxicity in vitro.

### Methods

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

AAT dosage. Normal human plasma contains 0.8-2.4 mg/ml AAT, with a half life of 5-6 days.

### Example 12

In one exemplary method it was demonstrated that AAT and HIAAT(ΔAAT) inhibit HIV production in chronically infected U1 cells.

U1 cells are derived from the U937 human monocytic cell line by the stable incorporation of 2 copies of HIV provirus into the cell genome. These cells generate increased HIV following exposure to any of several stimuli. In these exemplary experiments, U1 cells were cultured at a density of 1×10⁶ cells per ml in 500 μl of medium consisting of RPMI 1640 medium with 10% [vol/vol] heat inactivated fetal calf serum, with penicillin 100 units/ml+streptomycin 100 μg/ml. Cells were cultured in wells of a polystyrene tissue culture plate with medium alone (control), with medium containing stimulus alone (3 nM IL 18), or with stimulus in the presence of AAT (FIG. 15A, left panel) or heat inactivated AAT (FIG. 15A, right panel). AAT was added to cultures 1.0 hr prior to the addition of IL-18 (interleukin 18) stimulus. Cultures were incubated for 24 hrs (37° C., 5% CO₂), and then lysed with 1% (vol/vol) triton X 100 and then the lysates were assayed for HIV p24 antigen using an ELISA. As shown in FIGS. 15A and 15B, IL-18 stimulated an increase in HIV production compared to medium alone (control) cultures. Stimulating U1 cell cultures with IL-18 in the presence of either unaltered (FIG. 15A, left panel) AAT or with heat inactivated AAT (FIG. 15A, right panel) resulted in dose dependent inhibition of stimulated HIV production. Comparing native with heat inactivated AAT showed very similar inhibition of p24 production. For both native and heat inactivated AAT, nearly complete HIV suppression induced by IL 18 was observed using AAT concentrations of 4 and 6 mg/ml. These results suggest very similar HIV suppression in this chronic infection model using native or heat inactivated AAT. Another experiment was performed using 0.8 or 5 mg/ml of AAT or HI AAT (FIG. 15B). For both native and heat inactivated AAT, nearly complete HIV suppression induced by IL 18 was observed using AAT of HI AAT concentrations of 5 mg/ml but not at 0.8 mg/ml. Since heat inactivation of AAT using our protocol ablates AAT serine protease inhibitory function (as documented in by an in vitro serine protease neutralization assay, data not shown), these results suggest that AAT suppression of HIV in these studies does not depend on the serine protease inhibitor function of AAT.

### Example 13

### Methods

These experiments were performed with an exemplary strain of Influenza A, H1N1 Puerto Rico strain of influenza virus.

Due to the emergence of influenza variants, the ability of current vaccines to control viral infection and spread is limited. Hemagglutinin (HA) and neuraminidase are the two viral surface “spike” proteins involved in cellular viral uptake and new virion release, respectively. The HA protein must be proteolytically cleaved, yielding an infectious virion capable of binding terminal sialic acid residues on cell surface molecules, permitting uptake.

In these studies, in vitro (primary Rhesus monkey kidney cells) and in vivo (mouse) models were used to demonstrate that a serine protease inhibitor can inhibit influenza infection and production. In addition, clinical data indicate that AAT-deficiency is one risk factor for influenza infection.

### In Vitro Studies

In one exemplary method, influenza replication was demonstrated in cell culture. Primary Rhesus monkey kidney cells in vials (shell vial cultures) were incubated in a media, Zero Serum Re-feed media (ZSR), in the presence or absence of AAT, AAPV-CMK (synthetic inhibitor of host serine proteases), or human serum albumin, for 1 hr at 37° C. Tissue culture-adapted influenza A/PR/8/34 (H1N1) was added to the vials (final ˜900 TCID50) and incubated for an additional hour at 37° C. The infected supernatant was aspirated from the vials. The vials were rinsed once, and fresh 1-mL aliquots of ZSR AAT, AAPV-CMK, or albumin were added. Vials were incubated at 37° C. for 2 days and cell-free supernatants were collected and stored at −70° C. Nuclear Protein (reported as HA (hemagglutinin) units) was quantified in this example using an Influenza A virus EIA kit, other kits and methods are available. AAT inhibited primary monkey kidney cell infection with an H1N1 influenza strain by 77% and 59% in 2 and 3 day cultures, respectively, as determined by nuclear protein ELISA.

Immunohistochemistry. Shell vial cultures were pre-incubated as above but infected with 9000 TCID50 influenza A/PR/8/34 (H1N1), a ten-fold increase in inoculum. About, eighteen hours after infection, the medium was removed and the cells rinsed 2× with PBS. Cells were fixed for 5-10 min with chilled, 100% acetone, rinsed 1× with PBS, and stained at 37° C. for 15-30 min with DFA (fluoresceinated a-influenza mouse monoclonal) reagent.

### In Vivo Studies

Mouse weight loss and mortality studies were performed for in vivo analysis of AAT effects on influenza infection. A mouse model was used where huAAT+/+ mice (back-crossed to the C57BL/6 strain) express human AAT in the lung under control of the Surfactant C promoter. Weight-matched C57BL/6 wild-type (WT) mice were purchased from Jackson Labs. Animals anaesthetized with isoflurane were nasally challenged with a 50 mL suspension of 100 fluorescent focus units (FFU, influenza) mouse-adapted PR8 (low dose) for the weight loss studies or with 1000 FFU (high dose, influenza) for the mortality model. In these studies, mice that over-express human AAT in lung (AAT mice) exhibited significantly decreased baseline levels of the pro-inflammatory cytokines MIP-2, IL-6, and IL-1α and β compared to control C57BL/6 mice.

Lung inflation for histology was examined in mice exposed to the various units of influenza virus. Randomly selected mice were sacrificed (2×50 mL nembutal IP) on days 2, 3, and 4 post-influenza infection. Lungs of these mice were inflated and stored in 4% paraformaldehyde in PBS, pH 7.2 for 18-24 hours at 4° C., then transferred to PBS before sectioning for H&E staining (hematoxylin and eosin staining).

Caspase and cytokine studies. WT and huAAT+/+ mice were infected with high dose influenza. Animals were sacrificed on days 2, 3, and 4 post-infection and lungs harvested. One half of the left lung was used for caspase 1 and 3 analyses and the other half used for cytokine analyses. For caspase analyses, the cytosolic extract from renal cortex (200-400 mg protein) was incubated with the substrates Ac-YVAD-AMC (caspase-1) or Ac-DEVD-AMC (caspase-3).

Clinical Flu/AAT data

A transplantation clinic possesses a prospective observational database that follows all institutional lung transplant patients since 1 Feb. 1992. For this analysis, all patients who received lung transplants before 1 Apr. 2000 were included. Characteristics of the cohort included:

N=156 (excluded: N=16 died before 30 days of follow up or were lost to follow-up with none contracting Flu)-final cohort N=140; 79 male/61 female, median age in years=47.3 (range=14.1-65.1) Underlying disease categories include—Chronic obstructive pulmonary disease N=61 (44%); AAT deficiency N=28 (20%); Interstitial lung disease N=26 (19%); Cystic fibrosis N=20 (14%); Combined primary pulmonary hypertension and Eisenmenger's syndrome N=5. A Kaplan-Meier analysis was performed and graphed

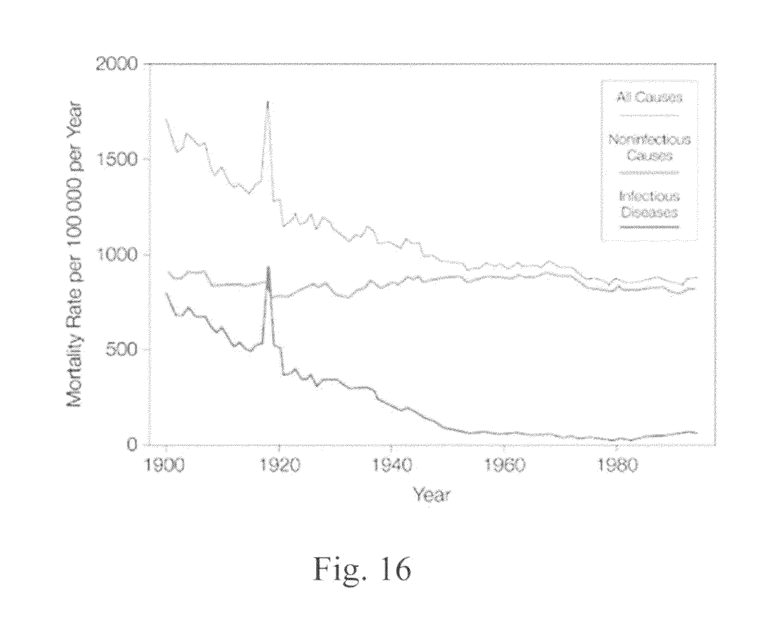

FIG. 16 illustrates a plot demonstrating the huge increase in incidence of influenza (H1N1) in 1918 resulting in death.

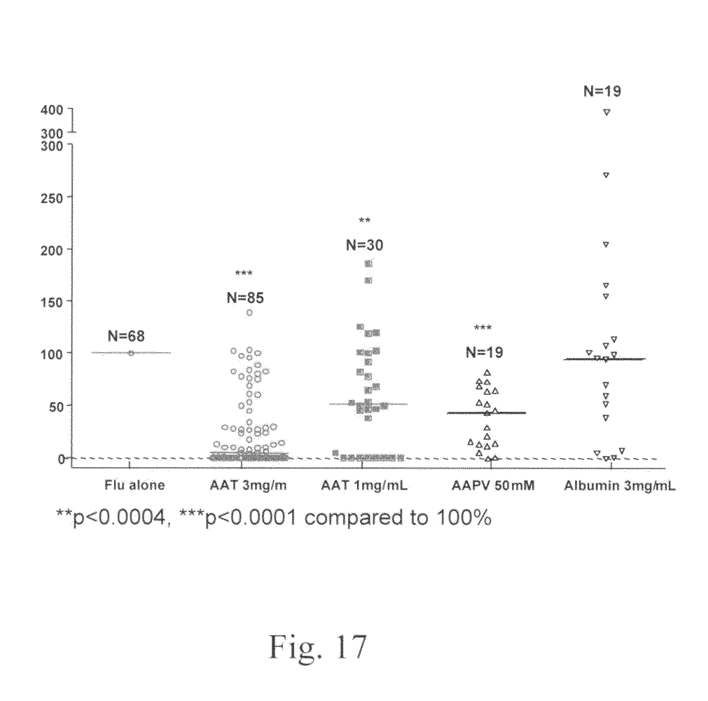

FIG. 17 represents a graphic illustration of the effect of increasing amounts of AAT on influenza production at Day 2 in vitro compared to controls, influenza alone and influenza in the presence of albumin. The number of samples in each condition is indicated. Effect of AAT on Influenza A infection of Rhesus monkey kidney cells was examined. Cells were pre-incubated for 1 hour in serum-free medium alone or with 3 or 1 mg/mL AAT, 50 mM AAPV, or 3 mg/mL human serum albumin (huAlb). Then the cultures were infected with influenza and supernatants were collected 48 hours later. Influenza production was measured by ELISA. Cells not pre-treated with an agent (influenza alone) were set to 100% infection, and all other treatments compared to these values. **p<0.0004, ***p<0.0001 compared to 100%. (e.g. Wilcoxin Signed Rank Test). This data illustrates that AAT can inhibit the release of influenza from an infected cell and potentially reduce the spread of influenza virus from one cell to another or perhaps one subject to another by reducing shedding of the virus. In addition, AAT may inhibit the intracellular production of influenza from the nucleus to the cytoplasm of the cell (see FIG. 17).

AAT inhibition of H1N1 influenza infection, both in vitro and in vivo. Addition of physiologically relevant doses (3 or 1 mg/mL) AAT decreased detectable influenza in day 2 of primary monkey kidney cell supernatants by about 76% and 40% respectively (FIG. 17). AlaAlaProVal-CMK (AAPV), a SERPIN mimic, also proved inhibitory at day 2, with a 62% mean reduction of influenza. AAPV inhibits serine protease activity, suggesting that one mechanism by which AAT inhibits influenza is by inhibiting HA cleavage on both the initial infecting particles as well as newly emergent virions, rendering them unable to initiate further rounds of infection.

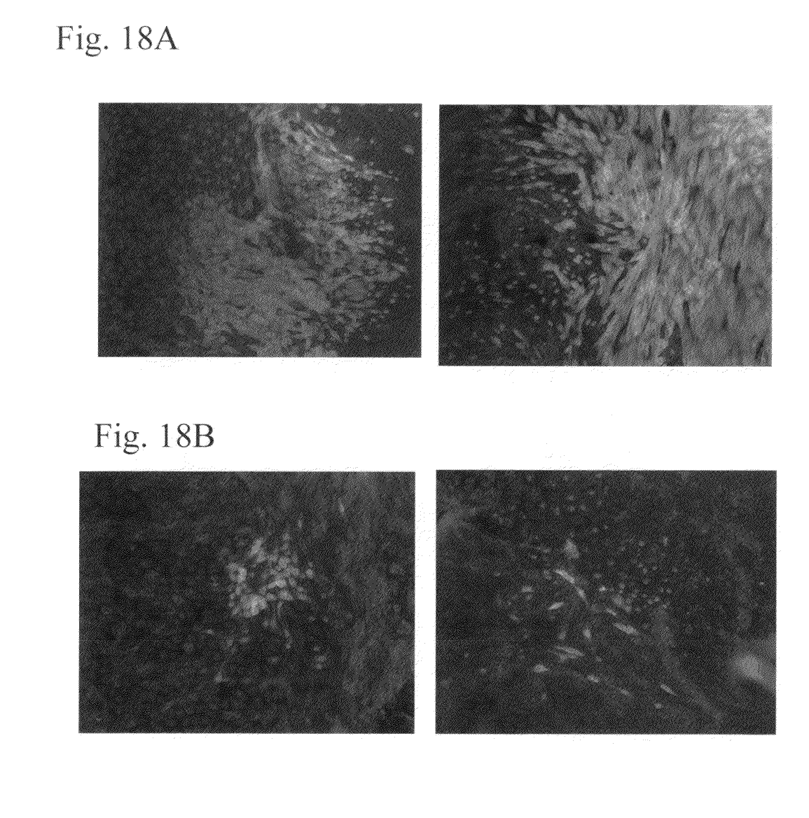

FIG. 18 represents fluorescence detection of influenza (e.g. H1N1) in an exemplary in vitro experiment A) represents influenza alone and B) represents influenza in the presence of an AAT composition disclosed herein. Effects of AAT on early Influenza A infection events in Rhesus monkey primary kidney cells were examined (see FIG. 18). Cells preincubated for 1 hour in serum-free medium alone (influenza alone), or with 3 mg/mL AAT were infected with influenza (9000 TCID50). After 18 hours the cells were fixed and stained with fluorescein-tagged anti-influenza. A antibodies. Representative images of influenza alone (FIG. 18A, representative of 10 fields) or influenza preincubated with 3 mg/mL AAT (FIG. 18B, representative of 15 fields) are demonstrated. Green staining indicates influenza infected cells.

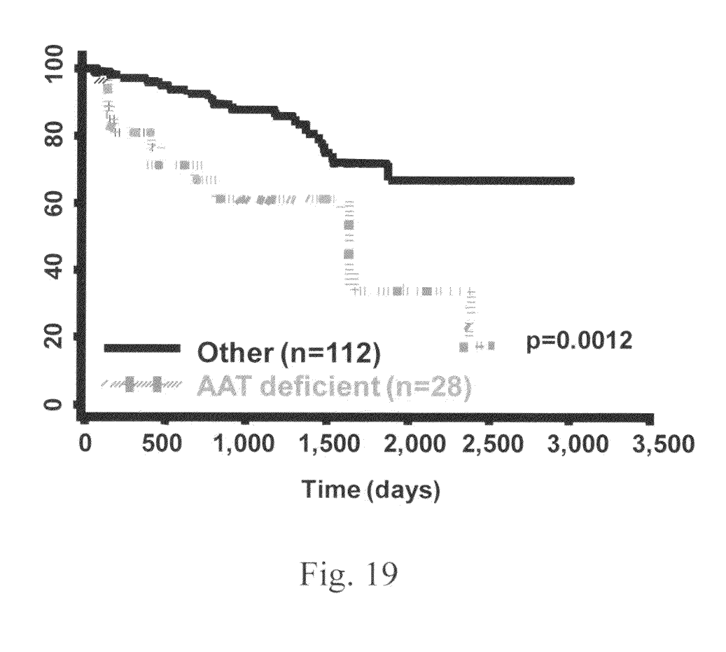

FIG. 19 represents a correlative exemplary plot of subjects having a reduced amount (n=28) of AAT compared to those having a relatively normal level (n=112) of AAT and increased risk of influenza over time (days). AAT deficiency is one risk factor for influenza infection in lung transplant patients. A Kaplan-Meier analysis in a prospectively-assessed cohort of lung transplant patients performed over approximately 8 years demonstrates significantly increased prevalence of influenza infection in lung transplant patients with AAT deficiency (N=28) compared to patient without AAT deficiency (N-112).

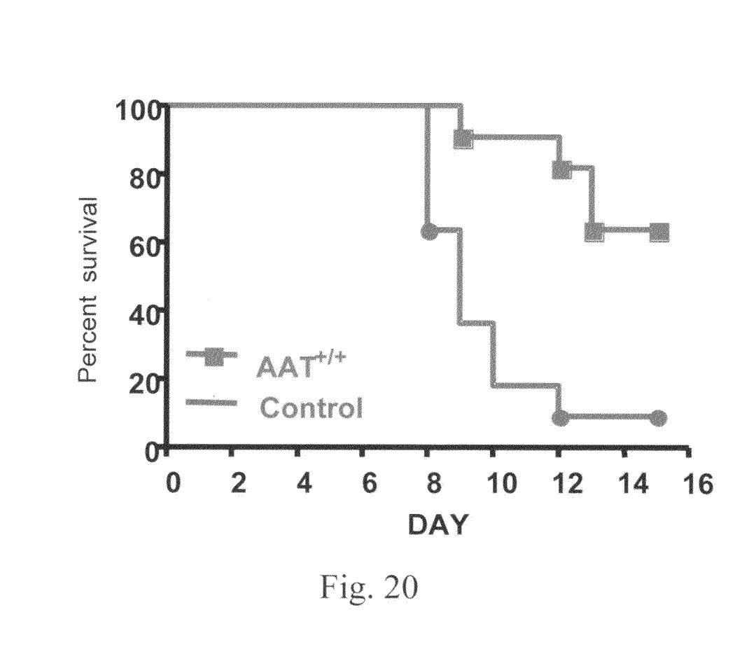

FIG. 20 represents an exemplary mouse model of influenza. Here, an in vivo assay was used to study a mouse population in the presence or absence of AAT and the percent survival of the mice over time after influenza (H1N1) infection. Transgenic mice expressing human AAT in the lungs were demonstrated to have increased protection from a lethal dose of influenza compared to control animals. Wild-type C57BL/6 mice (N=11) and huAAT+/+mice (N=11) were nasally challenged with a high dose (1000 FFU) of influenza. Mice were monitored daily and mortality plotted as group means with SEM. *p<0.007. This experiment demonstrated a statistically significant result in the mice having AAT compared to the control mice, p=0.0007. Nearly 60% of the mice having AAT lived through the 16-day test period versus less than about 10% without AAT.

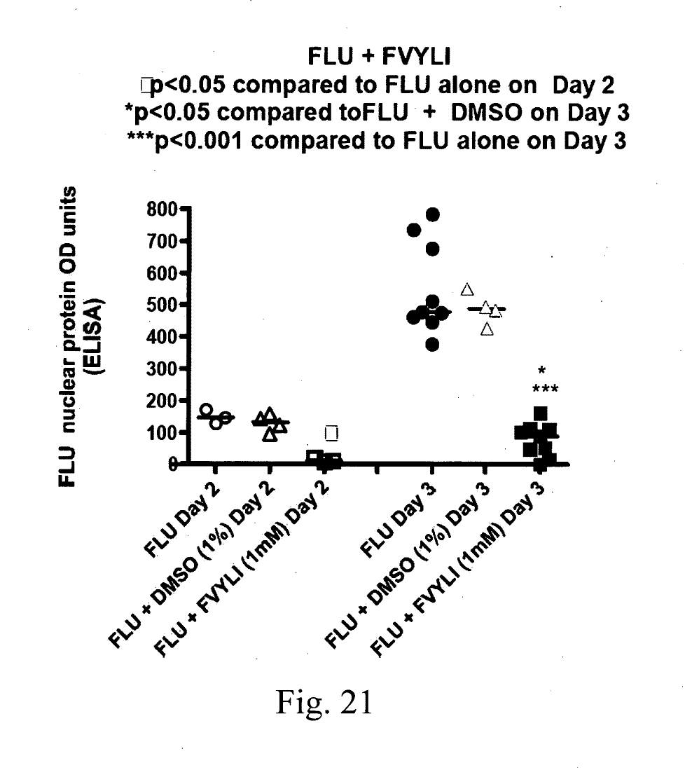

Adherent monkey kidney (MK) cell monolayers were grown in commercial shell vials in an incubator (5% CO2, 37° C.). On the day of experimentation, the monolayers were rinsed 1× with medium (Zero Serum Refeed or ZSR) and then pre-incubated for 1 hour with 230 uL of medium alone (Control), with medium containing DMSO control (final concentration 1% vol/vol), or with the FVYLI pentapeptide in DMSO (final FVYLI concentration=1 mM and final DMSO concentration of 1% vol/vol) (see FIG. 21).

Virus was then added to each shell vial (0.03 uL/vial in MK46 and 0.01 uL/vial in MK47) in 20 uL ZSR/culture and incubated for 1 hour. Infection medium was then aspirated and cells rinsed 1× with medium. Three hundred fifty uL of medium alone, medium with DMSO, or FVYLI was added to each vial and incubated for 3 days in an incubator (5% CO2/37° C.). An aliquot of supernatant was taken on day 2 of incubation from each culture and frozen at −70° C., and the remaining supernatants were collected and frozen at day 3. All culture supernatants were then assayed using an ELISA that quantifies the influenza nuclear protein.

Day 2 experiments included Control N=3, DMSO N=4, and FVYLI N=3. In Day 3 experiments, FLU alone N=9, DMSO alone N=5, and for FVYLI, N=9. Bars within the graph depict median values. FIG. 21 represents an exemplary graph of an experiment illustrating effects of a peptide FVYLI (SEQ. ID NO. 16) on influenza virus infection. The p-values are indicated on the figure for some of the conditions.

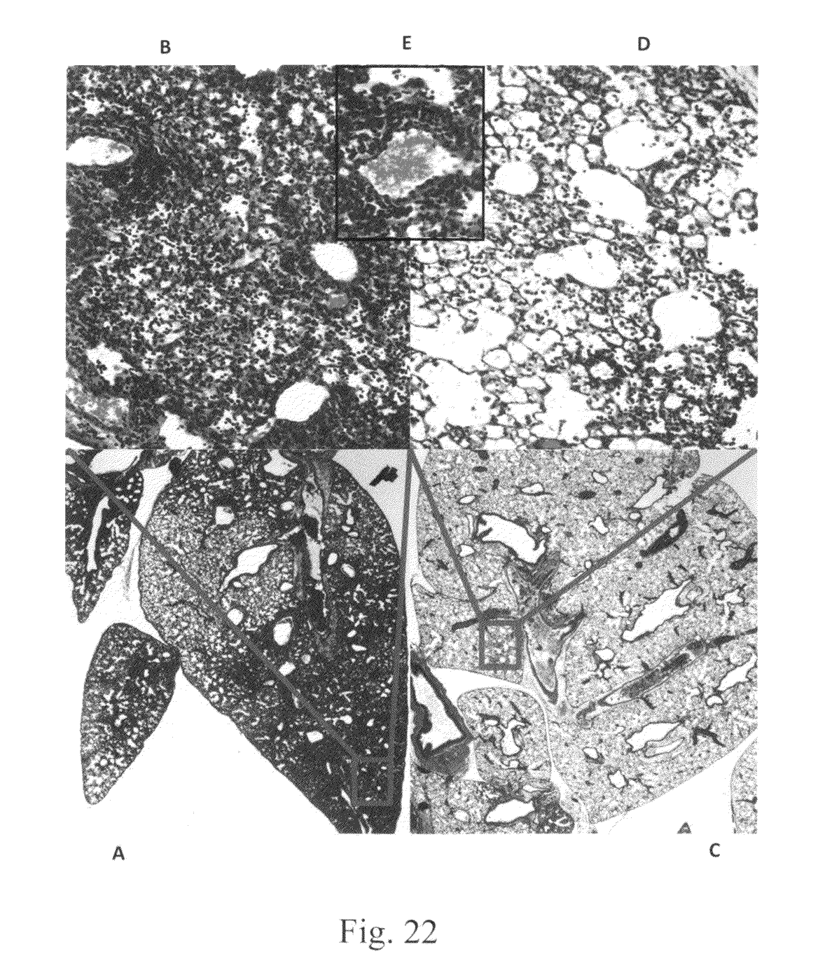

FIG. 22. Represents a pathology section of mice comparing pneumonia infiltrates in the presence or absence of AAT. Lobar pneumonia (A) with severe mixed acute and chronic inflammatory infiltrate (B) in wild type mouse. Characteristic patchy bronchopneumonia (C) with mild mixed acute and chronicinflammatory infiltrate (D) in transgenic α-1-antitrypsin overexpressing mouse. (E) Inset demonstrates perivascular cuffing with mononuclear predominant infiltrate common in influenza associated pneumonias. These features are present in both mouse models (note vascular cuffing in mid left regions of panels B and D). Magnification: A,C 20×; B,D 200×; Inset 600×.

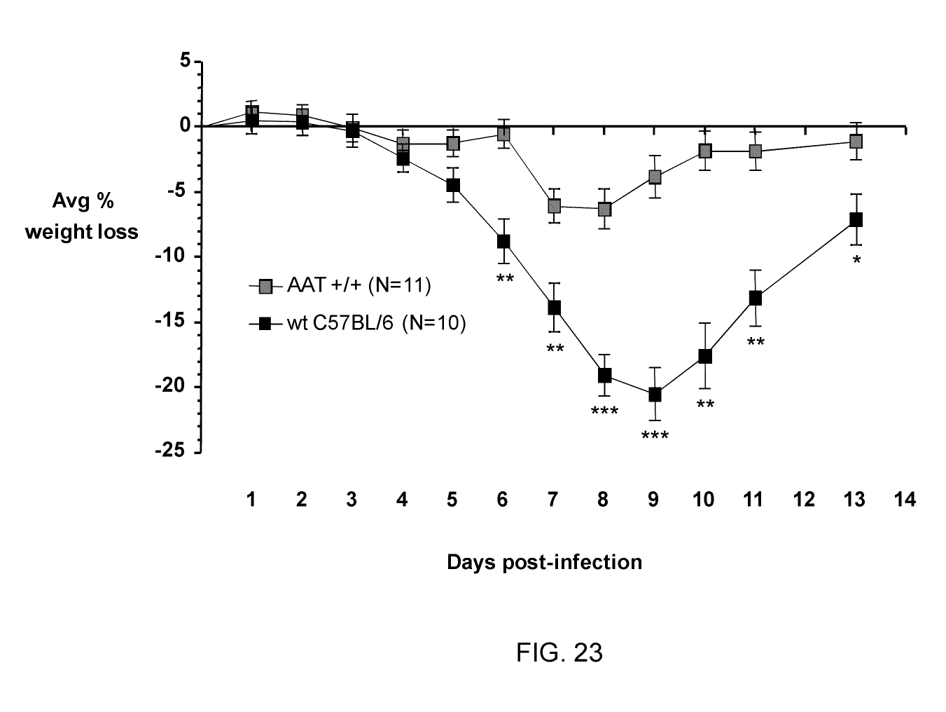

FIG. 23 represents transgenic mice expressing human AAT in the lungs experience reduced weight loss compared to WT (wild-type) controls during influenza infection. Wild-type C57BL/6 mice (N=10) and huAAT+/+mice (N=11) were nasally challenged with a low dose (100 FFU) of influenza. Individual weights were recorded daily, grouped, and graphed as mean % weight loss from starting weight. Group means with SEM were plotted. *p<0.05, **p<0.01, ***p<0.001. In this influenza pneumonia model, lung MIP-2 and IL-1α were reduced in AAT mice compared to C57BL/6 controls, and lung caspase-3 activity was 67% lower in AAT mice (p=0.004). Following influenza pneumonia, there were significant reductions in weight loss (morbidity) and mortality in AAT mice as compared to controls.

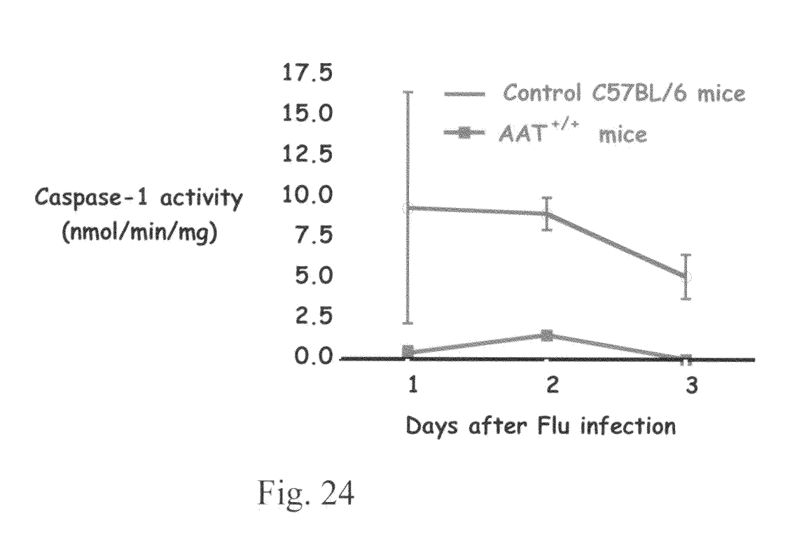

FIG. 24 represents caspase-1 levels in wild-type (control) compared to huAAT mice days after exposure to influenza. The top line (green) represents the wild-type animals and the lower line (red) represents the huAAT carrying animals. This data illustrates that expression of AAT correlates with reduction in Caspase-1 activity. These data in total and alone suggest a promising approach to influenza treatment for, and prevention of, infection that is safe and applicable as therapy for all variants of influenza, and is impervious to variations in influenza components due to antigenic shift or drift.

All of the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method for modulating shedding of a virus in a subject infected with the virus comprising, administering to the subject having a viral infection a therapeutically effective amount of a composition comprising one or more peptides derived from the carboxyterminal 80 amino acids of SEQ ID NO:20 corresponding to amino acid 315 and ending at amino acid 394, wherein the composition modulates shedding of the virus.

2. The method of claim 1, wherein the virus comprises HIV, AIDS (acquired immunodeficiency syndrome), influenza virus, hepatitis virus, Herpes virus, human papilloma virus, Variola major virus (small pox), Lassa fever virus, avian influenza, AIDS Related Complex, Chickenpox (Varicella), Common cold, Cytomegalovirus, Colorado tick-associated viruses, Dengue, Ebola virus, Hepatitis, Herpes simplex, Herpes zoster, HPV, Infectious mononucleosis-associated virus, PolioRabies, Rubella, SARS, Smallpox (Variola), pneumonia, West Nile, and a combination thereof.

3. The method of claim 1, wherein the composition comprises one or more peptide comprising FVFLM (SEQUENCE ID NO. 1), FVFAM (SEQUENCE ID NO. 2), FVALM (SEQUENCE ID NO. 3), FVFLA (SEQUENCE ID NO. 4), FLVFI (SEQUENCE ID NO. 5), FLMII (SEQUENCE ID NO. 6), FLFVL (SEQUENCE ID NO. 7), FLFVV (SEQUENCE ID NO. 8), FLFLI (SEQUENCE ID NO. 9), FLFFI (SEQUENCE ID NO. 10), FLMFI (SEQUENCE ID NO. 11), FMLLI (SEQUENCE ID NO. 12), FIIMU (SEQUENCE ID NO. 13), FLFCI (SEQUENCE ID NO. 14), FLFAV (SEQUENCE ID NO. 15), FVYLI (SEQUENCE ID NO. 16), FAFLM (SEQUENCE ID NO. 17), AVFLM (SEQUENCE ID NO. 18), and a mixture thereof.

4. The method of claim 1, wherein the composition comprises one or more peptide comprising GADLSGVTEE (SEQ ID NO:21); KAVLTIDEKG (SEQ ID NO:22); TEAAGAMFLE (SEQ ID NO:23); RIPVSIPPEV (SEQ ID NO:24); KFNKPFVFLM (SEQ ID NO:25); IEQNTKSPLF (SEQ ID NO:26); MGKVVNPTQK (SEQ ID NO:27); LSGVTEEAPL (SEQ. ID NO. 28); KLSKAVHKAV (SEQ. ID NO. 29); LTIDEKGTEA (SEQ. ID NO. 30); AGAMFLERIP (SEQ. ID NO. 31); VSIPPEVKFN (SEQ. ID NO. 32); KPFVFLMIEQ (SEQ. ID NO. 33); NTKSPLFMGK (SEQ. ID NO. 34); VVNPTQK (SEQ. ID NO. 35); LEAIPMSIPPEVKFNKPFVFLM (SEQ ID NO: 36); LEAIPMSIPPEVKFNKPFVF (SEQ ID NO: 37) or a mixture thereof.

5. The method of claim 1, wherein the virus is influenza.

6. The method of claim 6, wherein the composition comprises FVFLM (SEQUENCE ID NO. 1) or analog thereof, FVYLI (SEQUENCE ID NO. 16), LEAIPMSIPPEVKFNKPFVFLM (SEQ ID NO: 36); and LEAIPMSIPPEVKFNKPFVF (SEQ ID NO: 37) or a mixture thereof.

7. The method of claim 1, wherein the composition further comprises an agent selected from the group consisting of an anti-inflammatory agent, an immunosuppressive agent, an immunomodulatory agent, an anti-viral agent, an anti-pathogenic agent, an anti-bacterial agent, a reverse transcriptase inhibitor, a protease inhibitor, and a combination thereof.

8. The method of claim 1, wherein the composition is administered orally, systemically, via an implant, intravenously, topically, intrathecally, by inhalation, nasally or a combination thereof.

9. The method of claim 1, wherein the composition is administered by inhalation, nasally or a combination thereof.

10. A method for modulating shedding of a virus in a subject infected with the virus comprising, administering to the subject having a viral infection a therapeutically effective amount of a composition comprising SEQ ID NO:20, wherein the composition modulates shedding of the virus.

11. A composition for modulating shedding of a virus in a subject infected with the virus comprising, one or more peptides derived from the carboxyterminal 80 amino acids of SEQ ID NO:20 beginning at amino acid 315 and ending at amino acid 394, the composition modulates shedding of the virus from a cell.

12. The composition of claim 11, wherein the composition comprises FVFLM (SEQUENCE ID NO. 1), an analog of FVFLM comprising FVYLI (SEQUENCE ID NO. 16), or a mixture thereof.

13. The composition of claim 11, wherein the composition comprises GADLSGVTEE (SEQ ID NO:21); KAVLTIDEKG (SEQ ID NO:22); TEAAGAMFLE (SEQ ID NO:23); RIPVSIPPEV (SEQ ID NO:24); KFNKPFVFLM (SEQ ID NO:25); IEQNTKSPLF (SEQ ID NO:26); MGKVVNPTQK (SEQ ID NO:27); LSGVTEEAPL (SEQ. ID NO. 28); KLSKAVHKAV (SEQ. ID NO. 29); LTIDEKGTEA (SEQ. ID NO. 30); AGAMFLERIP (SEQ. ID NO. 31); VSIPPEVKFN (SEQ. ID NO. 32); KPFVFLMIEQ (SEQ. ID NO. 33); NTKSPLFMGK (SEQ. ID NO. 34); VVNPTQK (SEQ. ID NO. 35); LEAIPMSIPPEVKFNKPFVFLM (SEQ ID NO: 36); LEAIPMSIPPEVKFNKPFVF (SEQ ID NO: 37) or a mixture thereof.

14. The composition of claim 11, further comprises at least one of an anti-viral agent and an anti-pathogenic agent.

15. The composition of claim 11, wherein virus comprises influenza.

16. The composition of claim 15, wherein influenza virus comprises H1N1.

17. A kit for modulating shedding of a virus comprising:
at least one container; and
at least one composition comprising one or more of FVFLM (SEQUENCE ID NO. 1), an analog of FVFLM comprising FVYLI (SEQUENCE ID NO. 16), GADLSGVTEE (SEQ ID NO:21); KAVLTIDEKG (SEQ ID NO:22); TEAAGAMFLE (SEQ ID NO:23); RIPVSIPPEV (SEQ ID NO:24); KFNKPFVFLM (SEQ ID NO:25); IEQNTKSPLF (SEQ ID NO:26); MGKVVNPTQK (SEQ ID NO:27); LSGVTEEAPL (SEQ. ID NO. 28); KLSKAVHKAV (SEQ. ID NO. 29); LTIDEKGTEA (SEQ. ID NO. 30); AGAMFLERIP (SEQ. ID NO. 31); VSIPPEVKFN (SEQ. ID NO. 32); KPFVFLMIEQ (SEQ. ID NO. 33); NTKSPLFMGK (SEQ. ID NO. 34); VVNPTQK (SEQ. ID NO. 35); LEAIPMSIPPEVKFNKPFVFLM (SEQ ID NO: 36); LEAIPMSIPPEVKFNKPFVF (SEQ ID NO: 37) or a mixture thereof.

18. The kit of claim 17, wherein the composition comprises FVFLM (SEQUENCE ID NO. 1), an analog of FVFLM comprising FVYLI (SEQUENCE ID NO. 16), or a mixture thereof.

19. The kit of claim 17, further comprising an anti-viral agent.

20. A method for modulating influenza infection of cells of a subject comprising, administering to the subject having an influenza infection a therapeutically effective amount of a composition comprising SEQ ID NO:20 or one or more peptides derived from the carboxyterminal 80 amino acids of SEQ ID NO:20 corresponding to amino acid 315 and ending at amino acid 394, modulating hemagluttinin processing of influenza wherein modulating hemagluttinin processing of influenza reduces infectivity of influenza.
